SEQUENCE LISTING (85 sequences)

SEQ ID NO: 1 - DNA, 1338 bp, Arabidopsis
atgggccaccaaaacgccgccgtttcagagaatcaaaaccatgatgacggcgctgcgtcgtcgccgggattcaagctcgtcggattttccaagttcgtaagaaagaatccaaagtctgataaattcaaggttaagcgcttccatcacatcgagttctggtgcggcgacgcaaccaacgtcgctcgtcgcttctcctggggtctggggatgagattctccgccaaatccgatctttccaccggaaacatggttcacgcctcttacctactcacctccggtgacctccgattccttttcactgctccttactctccgtctctctccgccggagagattaaaccgacaaccacagcttctatcccaagtttcgatcacggctcttgtcgttccttcttctcttcacatggtctcggtgttagagccgttgcgattgaagtagaagacgcagagtcagctttctccatcagtgtagctaatggcgctattccttcgtcgcctcctatcgtcctcaatgaagcagttacgatcgctgaggttaaactatacggcgatgttgttctccgatatgttagttacaaagcagaagataccgaaaaatccgaattcttgccagggttcgagcgtgtagaggatgcgtcgtcgttcccattggattatggtatccggcggcttgaccacgccgtgggaaacgttcctgagcttggtccggctttaacttatgtagcggggttcactggttttcaccaattcgcagagttcacagcagacgacgttggaaccgccgagagcggtttaaattcagcggtcctggctagcaatgatgaaatggttcttctaccgattaacgagccagtgcacggaacaaagaggaagagtcagattcagacgtatttggaacataacgaaggcgcagggctacaacatctggctctgatgagtgaagacatattcaggaccctgagagagatgaggaagaggagcagtattggaggattcgacttcatgccttctcctccgcctacttactaccagaatctcaagaaacgggtcggcgacgtgctcagcgatgatcagatcaaggagtgtgaggaattagggattcttgtagacagagatgatcaagggacgttgcttcaaatcttcacaaaaccactaggtgacaggccgacgatatttatagagataatccagagagtaggatgcatgatgaaagatgaggaagggaaggcttaccagagtggaggatgtggtggttttggcaaaggcaatttctctgagctcttcaagtccattgaagaatacgaaaagactcttgaagccaaacagttagtgggatga

SEQ ID NO: 2 - protein, 445 aa, Arabidopsis
MGHQNAAVSENQNHDDGAASSPGFKLVGFSKFVRKNPKSDKFKVKRFHHIEFWCGDATNVARRFSWGLGMRFSAKSDLSTGNMVHASYLLTSGDLRFLFTAPYSPSLSAGEIKPTTTASIPSFDHGSCRSFFSSHGLGVRAVAIEVEDAESAFSISVANGAIPSSPPIVLNEAVTIAEVKLYGDVVLRYVSYKAEDTEKSEFLPGFERVEDASSFPLDYGIRRLDHAVGNVPELGPALTYVAGFTGFHQFAEFTADDVGTAESGLNSAVLASNDEMVLLPINEPVHGTKRKSQIQTYLEHNEGAGLQHLALMSEDIFRTLREMRKRSSIGGFDFMPSPPPTYYQNLKKRVGDVLSDDQIKECEELGILVDRDDQGTLLQIFTKPLGDRPTIFIEIIQRVGCMMKDEEGKAYQSGGCGGFGKGNFSELFKSIEEYEKTLEAKQLVG

SEQ ID NO: 3 - DNA, 1299 bp, Chlamydomonas
atgggcgctggtggcgcttctaccacggtagcgaatggcgggatcaagttggtagggcacaagaattttgtgcgctataatccacaatccgaccggtttgctattaagaggttccatagcttcgagttctggtgcgcggatgcgaccaacacatacaagcggttctcttatggcctgggcatgccgctggtcgccaagtccgaccagtccaccaacaaccagctctttgcctcctacgtgctgcgctccaacgacctggtcttcaccttcaccgcgccctacagccgcaagtgcgcctcggtcagcgagggcgttccgctgcgtcactacaacatcgaccatgcgtatgagttcatcaactcgcacgggctggcggtgcgggcagtaggcctgctggtggatgacgccaagacggcgtacgaggtgtctgtggcgcacggggccaagggcgtgctgccgccggtggagttgcgggatgaggcgagcggcaccagccaggtcatctcggaggtcattgtttacggggacgtcgttttccgctacgtgtcgggctccttcgagggccctttcatggccggctacacgccagtcacagactcgccggtcgcgtcgattgggttacagcgcgtggaccacgcggtgggcaacacacacgacctgatcaaggccgtggagtacatcaccgggttctgtggcttccacgagttctcagagtttgttgcggaggacgtgggcactgtggacagcggcctgaacagcatggtgcttgccaacaacgaggagaccatattgatgcctgtgaacgagcccaccttcggcacgccgcgcaagagccaaatccagacctacctggagcagaacgaggggccggggctgcagcacctggcgctgctcagcaacgacatcttcaccaccctgcgggagatgcgcgcgcgcagcgagctgggtggcttcgagttcatgccgcgggcaaatgcgaagtactacaaagacatgtacgcccgcatcggcgactcgctcacgccgcagcagtacagggaggtggaggagctgggcatcctggtggacaaggacgaccagggcgtgctgctgcagatcttcaccaagccgctgggcgaccggcccacggtgtttattgagatcatccagcgtgtgggctgcatgcgggaggtgaaggagcctgctacgggcgctgtggtggggacggagcaggcggctggctgcggcggcttcgggaaaggcaacttcggcgccctcttcaagtccattgaggactatgagcgcaccctaaatgtgtag

SEQ ID NO: 4 - protein, 432 aa, Chamydomonas
MGAGGASTTVANGGIKLVGHKNFVRYNPQSDRFAIKRFHSFEFWCADATNTYKRFSYGLGMPLVAKSDQSTNNQLFASYVLRSNDLVFTFTAPYSRKCASVSEGVPLRHYNIDHAYEFINSHGLAVRAVGLLVDDAKTAYEVSVAHGAKGVLPPVELRDEASGTSQVISEVIVYGDVVFRYVSGSFEGPFMAGYTPVTDSPVASIGLQRVDHAVGNTHDLIKAVEYITGFCGFHEFSEFVAEDVGTVDSGLNSMVLANNEETILMPVNEPTFGTPRKSQIQTYLEQNEGPGLQHLALLSNDIFTTLREMRARSELGGFEFMPRANAKYYKDMYARIGDSLTPQQYREVEELGILVDKDDQGVLLQIFTKPLGDRPTVFIEIIQRVGCMREVKEPATGAVVGTEQAAGCGGFGKGNFGALFKSIEDYERTLNV

SEQ ID NO: 5 - DNA, 1299 bp, Chlamydomonas
atgggagcgggtggtgcaggcaccggagatcgggaggggggcattaagctcgtgggctacaagaatttcgtgcgccagaacccgctttcagacaaattcaccgtccacaagtttcatcacatcgatttctggtgcggagatgcaacaaacacatcgaagcggttctcctacggcctgggcatgccgctggtcgccaagtccgaccagtccaccaacaaccagctctttgcctcctacgtgctgcgctccaacgacctggtcttcaccttcaccgcgccctacagccgcaagtgcgcctcggtcagcgagggcgttccgctgcgtcactacaacatcgaccatgcgtatgagttcatcaactcgcacgggctggcggtgcgggcagtaggcctgctggtggatgacgccaagacggcgtacgaggtgtctgtggcgcacggggccaagggcgtgctgccgccggtggagctgcgggatgaggcgagcggcaccagccaggtcatctcggaggtgctgctgtacggcgaggtcgtgctgcgctacgtgtcgggctccttccagggccccttcctggccggctacacgcccgtcacagactcggccgtgacctccttcggcctgcaacgtctggaccacgcggtgggcaacacccatgacctgatcaaggccgtggagtacatcaccggcttcacaggtttccacgagttctcagagtttgttgcggaggacgtgggcactgtggacagcggcctgaacagcatggtgctggcctccaacaacgaggcagtgctgctgcctgtgaacgagcccacctttggcacgccgcgcaagagccaaatccagacctacctggagcagaacgaggggccggggctgcagcacctggcgctgctcagcaacgacatcttcaccaccctgcgggagatgcgcgcgcgcagcgagctgggtggcttcgagttcatgccacgggcaaatgccaagtactacaaagacatgtacgcccgcatcggcgactcgctcacgccgcagcagtacagggaggtggaggagctgggcatcctggtggacaaggacgaccagggcgtgctgctgcagatcttcaccaagccgctgggcgaccggcccacggtgtttattgagatcatccagcgtgtgggctgcatgcgggaggtgaaagagcctgctacgggcgctgtggtggggacggagcaggcggctggctgcggcggcttcgggaaaggcaacttcggtgccctcttcaagtccattgaggactatgagcgcaccttaaatgtttaa

SEQ ID NO: 6 - protein, 432 aa, Chlamydomonas
MGAGGAGTGDREGGIKLVGYKNFVRQNPLSDKFTVHKFHHIDFWCGDATNTSKRFSYGLGMPLVAKSDQSTNNQLFASYVLRSNDLVFTFTAPYSRKCASVSEGVPLRHYNIDHAYEFINSHGLAVRAVGLLVDDAKTAYEVSVAHGAKGVLPPVELRDEASGTSQVISEVLLYGEVVLRYVSGSFQGPFLAGYTPVTDSAVTSFGLQRLDHAVGNTHDLIKAVEYITGFTGFHEFSEFVAEDVGTVDSGLNSMVLASNNEAVLLPVNEPTFGTPRKSQIQTYLEQNEGPGLQHLALLSNDIFTTLREMRARSELGGFEFMPRANAKYYKDMYARIGDSLTPQQYREVEELGILVDKDDQGVLLQIFTKPLGDRPTVFIEIIQRVGCMREVKEPATGAVVGTEQAAGCGGFGKGNFGALFKSIEDYERTLNV

SEQ ID NO: 7 - DNA, 1161 bp, Arabidopsis
atggagctctcgatctcacaatcaccgcgtgttcggttctcgtctctggcgcctcgtttcttagcagcttctcatcatcatcgtccttctgtgcatttagctgggaagtttataagcctccctcgagatgttcgcttcacgagcttatcaacttcaagaatgcggtccaaatttgtttcaaccaattatagaaaaatctcaatccgggcatgttctcaggttggtgctgctgagtctgatgatccagtgctggatagaattgcccggttccaaaatgcttgctggagatttcttagaccccatacaatccgcggaacagctttaggatccactgccttggtgacaagagctttgatagagaacactcatttgatcaaatggagtcttgtactaaaggcactttcaggtcttcttgctcttatttgtgggaatggttatatagtcggcatcaatcagatctacgacattggaatcgacaaagtgaacaaaccatacttgccaatagcagcaggagatctatcagtgcagtctgcttggttgttagtgatattttttgcgatagcagggcttttagttgtcggatttaactttggtccattcattacaagcctatactctcttggcctttttctgggaaccatctattctgttccacccctcagaatgaaaagattcccagttgcagcatttcttattattgccacggtacgaggtttccttcttaactttggtgtgtaccatgctacaagagctgctcttggacttccatttcagtggagtgcacctgtggcgttcatcacatcttttgtgacactgtttgcactggtcattgctattacaaaggaccttcctgatgttgaaggagatcgaaagttccaaatatcaaccctggcaacaaaacttggagtgagaaacattgcattcctcggttctggacttctgctagtaaattatgtttcagccatatcactagctttctacatgcctcaggtttttagaggtagcttgatgattcctgcacatgtgatcttggcttcaggcttaattttccagacatgggtactagaaaaagcaaactacaccaaggaagctatctcaggatattatcggtttatatggaatctcttctacgcagagtatctgttattccccttcctctag

SEQ ID NO: 8 - protein, 386 aa, Arabidopsis
MELSISQSPRVRFSSLAPRFLAASHHHRPSVHLAGKFISLPRDVRFTSLSTSRMRSKFVSTNYRKISIRACSQVGAAESDDPVLDRIARFQNACWRFLRPHTIRGTALGSTALVTRALIENTHLIKWSLVLKALSGLLALICGNGYIVGINQIYDIGIDKVNKPYLPIAAGDLSVQSAWLLVIFFAIAGLLVVGFNFGPFITSLYSLGLFLGTIYSVPPLRMKRFPVAAFLIIATVRGFLLNFGVYHATRAALGLPFQWSAPVAFITSFVTLFALVIAITKDLPDVEGDRKFQISTLATKLGVRNIAFLGSGLLLVNYVSAISLAFYMPQVFRGSLMIPAHVILASGLIFQTWVLEKANYTKEAISGYYRFIWNLFYAEYLLFPFL

SEQ ID NO: 9 - DNA, 1113 bp, Chlamydomonas
atggacctttgcagctcaactggaagaggagcatgcctttcgccggcatccacgtcgcggccgtgcccagcaccagtgcatttgcgcggccgacgcctggctttctctccggctcagcctgctggacggcgccacttgccggtgctctcatctgcagcggtccccgctcccctcccaaatggtggaaacgacgagagcttcgcacaaaaactggctaactttccaaacgccttctggaagttcctgcggccacacaccatccgggggactatcctgggcaccacagctgtgaccgccaaggtccttatggagaaccccggctgcatagactgggcactgctgccgaaggcgctgctcggcctggtggcgctgctgtgcggcaacggctacattgtgggcatcaaccaaatctacgacgtcgacattgacgtggtcaacaagccattcctccccgtggcgtcgggcgagctgtcgccggcgctggcgtggggcctgtgtctgtcgctggcggctgcgggcgcgggcatcgtagccgccaacttcggcaacctcatcaccagcctctacacctttggcctcttcctgggcaccgtgtacagtgtgcctcccctgcgcctgaagcagtacgcggtgccggccttcatgatcatcgccacggtgcgcggcttcctgctcaacttcggcgtgtacagcgccacgcgggcggcactgggactgcccttcgagtggagcccggccgtcagcttcatcacggtgtttgtgacgctgtttgccactgtgatcgccatcaccaaggacctgccggacgtggagggcgaccaggccaacaacatctccaccttcgccacgcgcatgggcgtgcgcaacgtggcactgctggccatcggccttctcatggccaactacctgggtgccatcgcgctggcactcacctactccaccgccttcaacgtgccgctcatggcgggcgcgcacgccatcctggccgccacgctggcgctgcgcacgctcaagctgcacgccgccagctacagccgggaggcggtggcgtccttctaccgctggatctggaacctgttctacgccgagtacgcgctgctgccgttcctgtag

SEQ ID NO: 10 - protein, 370 aa, Chlamydomonas
MDLCSSTGRGACLSPASTSRPCPAPVHLRGRRLAFSPAQPAGRRHLPVLSSAAVPAPLPNGGNDESFAQKLANFPNAFWKFLRPHTIRGTILGTTAVTAKVLMENPGCIDWALLPKALLGLVALLCGNGYIVGINQIYDVDIDVVNKPFLPVASGELSPALAWGLCLSLAAAGAGIVAANFGNLITSLYTFGLFLGTVYSVPPLRLKQYAVPAFMIIATVRGFLLNFGVYSATRAALGLPFEWSPAVSFITVFVTLFATVIAITKDLPDVEGDQANNISTFATRMGVRNVALLAIGLLMANYLGAIALALTYSTAFNVPLMAGAHAILAATLALRTLKLHAASYSREAVASFYRWIWNLFYAEYALLPFL

SEQ ID NO: 11 - protein, 433 aa, Physcomitrella
MGLDKSESEGSVVGPLHLVGCERFVRNNPKTDRFGVERFHHVEFWCGDASNTWRRFSWGLGMHLVAKSDQTTGNQTYCSYAIQSNELVFAFTAPYSSTIDQTNTKMPHPGYKSDEARSFTDSHGLAVRAVGILVDDADEAFRISVEHGAVSVLEPHVLSDDAKGGKMVMAEVKLYGDVVLRYVSEQGYKGSMLPNYEEVESLPLSYGLVRLDHAVGNVHNLAEAVNYIAKFTGFHEFAEFTAGDVGTTESGLNSMVVASNNEMVLLPINEPTFGTKRKSQIQTYLEHNEGPGLQHLALICDNIFSTLREMRTRTHIGGFDFMPKPPPTYYKNLANRVGDILTAEQIKECDELGILVDKDDQGVLLQIFTKPVGDRPSIFVEIIQRIGCMDKDESTGATVQKGGCGGFGKGNFSELFKSIEEYEKTLDGTLKVH

SEQ ID NO: 12 - protein, 446 aa, Oryza
MPPTPTPTATTGAVSAAAAAGENAGFRLVGHRRFVRANPRSDRFQALAFHHVELWCADAASAAGRFAFALGAPLAARSDLSTGNSAHASLLLRSASVAFLFTAPYGGDHGVGADAATTASIPSFSPGAARRFAADHGLAVHAVALRVADAADAFRASVAAGARPAFQPADLGGGFGLAEVELYGDVVLRFVSHPDGADAPFLPGFEGVSNPGAVDYGLRRFDHVVGNVPELAPVAAYISGFTGFHEFAEFTAEDVGTAESGLNSVVLANNAETVLLPLNEPVHGTKRRSQIQTYLDHHGGPGVQHIALASDDVLGTLREMRARSAMGGFEFLAPPPPNYYDGVRRRAGDVLSEEQINECQELGVLVDRDDQGVLLQIFTKPVGDRPTFFLEMIQRIGCMEKDESGQEYQKGGCGGFGKGNFSELFKSIEEYEKSLEAKQAPTVQGS

SEQ ID NO: 13 - protein, 436 aa, Triticum
MPPTPTTPAATGAGAAAAVTPEHARPRRMVRFNPRSDRFHTLSFHHVEFWCADAASAAGRFAFALGAPLAARSDLSTGNSVHASQLLRSGNLAFLFTAPYANGCDAATASLPSFSADAARRFSADHGLAVRSIALRVADAAEAFRASVDGGARPAFSPVDLGRGFGFAEVELYGDVVLRFVSHPDDTDVPFLPGFEGVSNPDAVDYGLTRFDHVVGNVPELAPAAAYVAGFAGFHEFAEFTTEDVGTAESGLNSMVLANNSEGVLLPLNEPVHGTKRRSQIQTFLEHHGGSGVQHIAVASSDVLRTLREMRARSAMGGFDFLPPRCRKYYEGVRRIAGDVLSEAQIKECQELGVLVDRDDQGVLLQIFTKPVGDRPTLFLEMIQRIGCMEKDERGEEYQKGGCGGFGKGNFSELFKSIEDYEKSLEAKQSAAVQGS

SEQ ID NO: 14 - protein, 444 aa, Zea
MPPTPTAAAAGAAVAAASAAEQAAFRLVGHRNFVRFNPRSDRFHTLAFHHVELWCADAASAAGRFSFGLGAPLAARSDLSTGNSAHASLLLRSGSLSFLFTAPYAHGADAATAALPSFSAAAARRFAADHGLAVRAVALRVADAEEAFRTSVAAGARPAFGPVDLGRGFRLAEVELYGDVVLRYVSYPDGAAGEPFLPGFEGVASPGAADYGLSRFDHIVGNVPELAPAAAYFAGFTGFHEFAEFTTEDVGTAESGLNSMVLANNSENVLLPLNEPVHGTKRRSQIQTFLDHHGGPGVQHMALASDDVLRTLREMQARSAMGGFEFMAPPTSDYYDGVRRRAGDVLTEAQIKECQELGVLVDRDDQGVLLQIFPKPVGDRPTLFLEIIQRIGCMERDEKGQEYQKGGCGGFGKGNFSQLFKSIEDYEKSLEAMQAAAAATAQGS

SEQ ID NO: 15 - protein, 443 aa, Glycine
  misc_feature (393)..(393): Xaa can be any naturally occurring amino acid
MCNEIQAQAQAQAQPGFKLVGFKNFVRTNPKSDRFQVNRFHHIEFWCTDATNASRRFSWGLGMPIVAKSDLSTGNQIHASYLLRSGDLSFLFSAPYSPSLSAGSSAASSASIPSFDAATCLAFAAKHGFGVRAIALEVADAEAAFSASVAKGAEPASPPVLVDDRTGFAEVRLYGDVVLRYVSYKDAAPQAPHADPSRWFLPGFEAAASSSSFPELDYGIRRLDHAVGNVPELAPAVRYLKGFSGFHEFAEFTAEDVGTSESGLNSVVLANNSETVLLPLNEPVYGTKRKSQIETYLEHNEGAGVQHLALVTHDIFTTLREMRKRSFLGGFEFMPSPPPTYYANLHNRAADVLTVDQIKQCEELGILVDRDDQGTLLQIFTKPVGDRPTIFIXIIQRIGCMVEDEEGKVYQKGACGGFGKGNFSELFKSIEEYEKTLEAKRTA

SEQ ID NO: 16 - protein, 445 aa, Vitis
MGKQNTTTNNPAPGFKLVGFSNFLRTNPMSDRFGVKRFHHIEFWSTDATNLARRFSWGLGMPIVAKSDLSTGNVIHASYLTRSGDLNFLFTAPYSPSIAGDLENAAATASIPSFDHSACHAFAASHGLGVRAIAIEVDDAEGAFHTSVAHGARPMSPPVTMGGSVVISEVHLYGDAVLRYVSYKNPNPNATSDPSSWFLPGFEAVDEGSSFPVDFGLRRVDHTVGNVPKLAPVVTYLKQFTGFHEFAEFTAEDVGTSESGLNSVVLASNNEMVLLPLNEPVFGTKRKSQIQTYLEHNEGPGVQHLALMSDDIFRTLREMRRRSGVGGFDFMPSPPPTYYRNVKKRAGDVLTDDQIKECEELGILVDKDDQGTLLQIFTKPLGDRPTIFIEIIQRLGCMVKDDEGKVSQKGGCGGFGKGNFSELFKSIEEYEKTLGAKRIVDPAPV

SEQ ID NO: 17 - protein, 358 aa, Pseudomonas fluorescens
MADQYENPMGLMGFEFIEFASPTPGTLEPIFEIMGFTKVATHRSKNVHLYRQGEINLILNNQPDSLASYFAAEHGPSVCGMAFRVKDSQQAYNRALELGAQPIHIETGPMELNLPAIKGIGGAPLYLIDRFGEGSSIYDIDFVYLEGVDRNPVGAGLKVIDHLTHNVYRGRMAYWANFYEKLFNFREARYFDIKGEYTGLTSKAMSAPDGMIRIPLNEESSKGAGQIEEFLMQFNGEGIQHVAFLTEDLVKTWDALKKIGMRFMTAPPDTYYEMLEGRLPNHGEPVDQLQARGILLDGSSIEGDKRLLLQIFSETLMGPVFFEFIQRKGDDGFGEGNFKALFESIERDQVRRGVLTTD

SEQ ID NO: 18 - protein, 358 aa, Pseudomonas fluorescens
MADLYENPMGLMGFEFIELASPTPNTLEPIFEIMGFTKVATHRSKDVHLYRQGAINLILNNEPHSVASYFAAEHGPSVCGMAFRVKDSQKAYKRALELGAQPIHIETGPMELNLPAIKGIGGAPLYLIDRFGEGSSIYDIDFVFLEGVDRHPVGAGLKIIDHLTHNVYRGRMAYWANFYEKLFNFREIRYFDIKGEYTGLTSKAMTAPDGMIRIPLNEESSKGAGQIEEFLMQFNGEGIQHVAFLSDDLIKTWDHLKSIGMRFMTAPPDTYYEMLEGRLPNHGEPVGELQARGILLDGSSESGDKRLLLQIFSETLMGPVFFEFIQRKGDDGFGEGNFKALFESIERDQVRRGVLSTD

SEQ ID NO: 19 - protein, 440 aa, Avena sativa
MPPTPATATGAAAAAVTPEHAARSFPRVVRVNPRSDRFPVLSFHHVELWCADAASAAGRFSFALGAPLAARSDLSTGNSAHASLLLRSGALAFLFTAPYAPPPQEAATAAATASIPSFSADAARTFAAAHGLAVRSVGVRVADAAEAFRVSVAGGARPAFAPADLGHGFGLAEVELYGDVVLRFVSYPDETDLPFLPGFERVSSPGAVDYGLTRFDHVVGNVPEMAPVIDYMKGFLGFHEFAEFTAEDVGTTESGLNSVVLANNSEAVLLPLNEPVHGTKRRSQIQTYLEYHGGPGVQHIALASNDVLRTLREMRARTPMGGFEFMAPPQAKYYEGVRRIAGDVLSEEQIKECQELGVLVDRDDQGVLLQIFTKPVGDRPTFFLEMIQRIGCMEKDEVGQEYQKGGCGGFGKGNFSELFKSIEDYEKSLEVKQSVVAQKS

SEQ ID NO: 20 - DNA, 16 bp, Artificial
  Primer
ggccaccaaaacgccg

SEQ ID NO: 21 - DNA, 24 bp, Artificial
  Primer
tcatcccactaactgtttggcttc

SEQ ID NO: 22 - DNA, 23 bp, Artificial
  Primer
ggcgctggcggtgcgtccactac

SEQ ID NO: 23 - DNA, 34 bp, Artificial
  Primer
tcaaacgttcagggtacgctcgtagtcttcgatg

SEQ ID NO: 24 - DNA, 21 bp, Artificial
  Primer
ggtgcgggtggcgctggcacc

SEQ ID NO: 25 - DNA, 35 bp, Artificial
  Primer
tcaaacgttcagggtacgttcgtagtcctcgatgg

SEQ ID NO: 26 - DNA, 19 bp, Artificial
  Primer
ccaatcccaatgtgcaacg

SEQ ID NO: 27 - DNA, 22 bp, Artificial
  Primer
ttatgcggtacgtttagcctcc

SEQ ID NO: 28 - DNA, 23 bp, Artificial
  Primer
ccaccgactccgaccgccgcagc

SEQ ID NO: 29 - DNA, 26 bp, Artificial
  Primer
tcaggaaccctgtgcagctgccgcag

SEQ ID NO: 30 - DNA, 17 bp, Artificial
  Primer
ccgccgactccaacccc

SEQ ID NO: 31 - DNA, 21 bp, Artificial
  Primer
ttaagaaccctgaacggtcgg

SEQ ID NO: 32 - DNA, 28 bp, Artificial
  Primer
gaggattcgacttcgcgccttctcctcc

SEQ ID NO: 33 - DNA, 28 bp, Artificial
  Primer
ggaggagaaggcgcgaagtcgaatcctc

SEQ ID NO: 34 - DNA, 29 bp, Artificial
  Primer
gaggattcgacttctggccttctcctccg

SEQ ID NO: 35 - DNA, 29 bp, Artificial
  Primer
cggaggagaaggccagaagtcgaatcctc

SEQ ID NO: 36 - DNA, 31 bp, Artificial
  Primer
ggaggattcgacttctttccttctcctccgc

SEQ ID NO: 37 - DNA, 31 bp, Artificial
  Primer
gcggaggagaaggaaagaagtcgaatcctcc

SEQ ID NO: 38 - DNA, 35 bp, Artificial
  Primer
gtgacaggccgacgatagctatagagataatccag

SEQ ID NO: 39 - DNA, 35 bp, Artificial
  Primer
ctggattatctctatagctatcgtcggcctgtcac

SEQ ID NO: 40 - DNA, 30 bp, Artificial
  Primer
gacttcatgcctcctcctccgcctacttac

SEQ ID NO: 41 - DNA, 30 bp, Artificial
  Primer
gtaagtaggcggaggaggaggcatgaagtc

SEQ ID NO: 42 - DNA, 31 bp, Artificial
  Primer
gattcgacttcatggcttctcctccgcctac

SEQ ID NO: 43 - DNA, 31 bp, Artificial
  Primer
gtaggcggaggagaagccatgaagtcgaatc

SEQ ID NO: 44 - DNA, 34 bp, Artificial
  Primer
cagatcaaggagtgtcaggaattagggattcttg

SEQ ID NO: 45 - DNA, 34 bp, Artificial
  Primer
caagaatccctaattcctgacactccttgatctg

SEQ ID NO: 46 - DNA, 38 bp, Artificial
  Primer
cggaacaaagaggaagagtgagattcagacgtatttgg

SEQ ID NO: 47 - DNA, 38 bp, Artificial
  Primer
ccaaatacgtctgaatctcactcttcctctttgttccg

SEQ ID NO: 48 - DNA, 40 bp, Artificial
  Primer
cgttgcttcaaatcttcccgaaaccactaggtgacaggcc

SEQ ID NO: 49 - DNA, 40 bp, Artificial
  Primer
ggcctgtcacctagtggtttcgggaagatttgaagcaacg

SEQ ID NO: 50 - DNA, 38 bp, Artificial
  Primer
caaatcttcacaaaaccagtgggtgacaggccgacgat

SEQ ID NO: 51 - DNA, 38 bp, Artificial
  Primer
atcgtcggcctgtcacccactggttttgtgaagatttg

SEQ ID NO: 52 - DNA, 40 bp, Artificial
  Primer
tgacaggccgacgatatttctggagataatccagagagta

SEQ ID NO: 53 - DNA, 40 bp, Artificial
  Primer
tactctctggattatctccagaaatatcgtcggcctgtca

SEQ ID NO: 54 - DNA, 30 bp, Artificial
  Primer
gacttcatgcctgcgcctccgcctacttac

SEQ ID NO: 55 - DNA, 30 bp, Artificial
  Primer
gtaagtaggcggaggcgcaggcatgaagtc

SEQ ID NO: 56 - DNA, 34 bp, Artificial
  Primer
ggcaatttctctgagttcttcaagtccattgaag

SEQ ID NO: 57 - DNA, 34 bp, Artificial
  Primer
cttcaatggacttgaagaactcagagaaattgcc

SEQ ID NO: 58 - DNA, 37 bp, Artificial
  Primer
ggaacaaagaggaagagtgtgattcagacgtatttgg

SEQ ID NO: 59 - DNA, 37 bp, Artificial
  Primer
ccaaatacgtctgaatcacactcttcctctttgttcc

SEQ ID NO: 60 - DNA, 28 bp, Artificial
  Primer
gaggattcgacttcaacccttctcctcc

SEQ ID NO: 61 - DNA, 28 bp, Artificial
  Primer
ggaggagaagggttgaagtcgaatcctc

SEQ ID NO: 62 - DNA, 28 bp, Artificial
  Primer
gaggattcgacttccagccttctcctcc

SEQ ID NO: 63 - DNA, 28 bp, Artificial
  Primer
ggaggagaaggctggaagtcgaatcctc

SEQ ID NO: 64 - DNA, 37 bp, Artificial
  Primer
ggaacaaagaggaagagtaacattcagacgtatttgg

SEQ ID NO: 65 - DNA, 37 bp, Artificial
  Primer
ccaaatacgtctgaatgttactcttcctctttgttcc

SEQ ID NO: 66 - DNA, 37 bp, Artificial
  Primer
ggaacaaagaggaagagtcacattcagacgtatttgg

SEQ ID NO: 67 - DNA, 37 bp, Artificial
  Primer
ccaaatacgtctgaatgtgactcttcctctttgttcc

SEQ ID NO: 68 - DNA, 23 bp, Artificial
  Primer
atgggcgctggtggcgcttctac

SEQ ID NO: 69 - DNA, 28 bp, Artificial
  Primer
ctacacatttagggtgcgctcatagtcc

SEQ ID NO: 70 - DNA, 23 bp, Artificial
  Primer
atgggagcgggtggtgcaggcac

SEQ ID NO: 71 - DNA, 30 bp, Artificial
  Primer
ttaaacatttaaggtgcgctcatagtcctc

SEQ ID NO: 72 - DNA, 26 bp, Artificial
  Primer
atggacctttgcagctcaactggaag

SEQ ID NO: 73 - DNA, 25 bp, Artificial
  Primer
gtacgcgctgctgccgttcctgtag

SEQ ID NO: 74 - DNA, 20 bp, Artificial
  Primer
mgbaarwsycagatycagac

SEQ ID NO: 75 - DNA, 20 bp, Artificial
  Primer
asnggyttngtraavayctg

SEQ ID NO: 76 - DNA, 29 bp, Artificial
  Primer
tggmgnttyytnmgnccncayacnathmg

SEQ ID NO: 77 - DNA, 29 bp, Artificial
  Primer
ytcngcnnhraanarrttccadatvmanc

SEQ ID NO: 78 - DNA, 26 bp, Artificial
  Primer
wsnggnytnaaywsnryngtnytngc

SEQ ID NO: 79 - DNA, 26 bp, Artificial
  Primer
raarttnccyttnccraanccnccrc

SEQ ID NO: 80 - DNA, 29 bp, Artificial
  Primer
tggmgnttyytnmgnccncayacnathmg

SEQ ID NO: 81 - DNA, 29 bp, Artificial
  Primer
ytcngcnnhraanarrttccadatvmanc

SEQ ID NO: 82 - DNA, 20 bp, Artificial
  Primer
atgggccaccaaaacgccgc

SEQ ID NO: 83 - DNA, 27 bp, Artificial
  Primer
tcatcccactaactgtttggcttcaag

SEQ ID NO: 84 - DNA, 23 bp, Artificial
  Primer
atggagctctcgatctcacaatc

SEQ ID NO: 85 - DNA, 27 bp, Artificial
  Primer
ctagaggaaggggaataacagatactc

(19) United States
(12) Patent Application Publication
(10) Pub. No.: US 2013/0053243 A1
(43) Pub. Date: Feb. 28, 2013

(54) PLANTS HAVING INCREASED TOLERANCE TO HERBICIDES

(71) Applicants: Thomas Mietzner, Annweiler (DE); Matthias Witschel, Bad Durkheim (DE); Johannes Hutzler, Waldsee (DE); Thomas Ehrhardt, Speyer (DE); Stefan Tresch, Kirchheim (DE)

(72) Inventors: Thomas Mietzner, Annweiler (DE); Matthias Witschel, Bad Durkheim (DE); Johannes Hutzler, Waldsee (DE); Thomas Ehrhardt, Speyer (DE); Stefan Tresch, Kirchheim (DE)

(73) Assignee: BASF SE, Ludwigshafen (DE)

(21) Appl. No.: 13/695,973

(22) PCT Filed: May 2, 2011
(86) PCT No.: PCT/IB11/51919

§ 371 Date: Nov. 7, 2012

Related U.S. Application Data
(60) Provisional application No. 61/330,922, filed on May 4, 2010.

(30) Foreign Application Priority Data
May 4, 2010 (EP) 10161867.6

(51) Int. Cl. A01H 5/00; C40B 30/06; C12N 15/53; A01P 13/00; A01H 5/10; C12N 15/82; C12Q 1/26; C12Q 1/48; A01N 43/90; C12N 5/10

(52) U.S. Cl. 504/130; 504/246; 506/10; 536/23.2; 435/418; 800/300; 435/468; 800/278; 435/25; 435/15

(57) ABSTRACT

The present invention refers to a method for controlling undesired vegetation at a plant cultivation site. The method comprises the steps of providing, at said site, a plant that comprises at least one nucleic acid comprising a nucleotide sequence encoding a wild-type hydroxyphenyl pyruvate dioxygenase or a mutated hydroxyphenyl pyruvate dioxygenase (mut-HPPD) which is resistant or tolerant to a coumarone-derivative herbicide and/or a nucleotide sequence encoding a wild-type homogentisate solanesyl transferase or a mutated homogentisate solanesyl tranferase (mut-HST) which is resistant or tolerant to a coumarone derivative herbicide, and then applying an effective amount of said herbicide to said plant cultivation site. The invention further refers to plants comprising mut-HPPD and to methods of obtaining such plants.

### FIELD OF THE INVENTION

The present invention relates in general to methods for conferring on plants agricultural level tolerance to an herbicide. Particularly, the invention refers to plants having an increased tolerance to “coumarone-derivative” herbicides. More specifically, the present invention relates to methods and plants obtained by mutagenesis and cross-breeding and transformation that have an increased tolerance to “coumarone-derivative” herbicides.

### BACKGROUND OF THE INVENTION

Herbicides that inhibit 4-hydroxyphenylpyruvate dioxygenase (4-HPPD; EC 1.13.11.27), a key enzyme in the biosynthesis of the prenylquinones plastoquinone and tocopherols, have been used for selective weed control since the early 1990s. They block the conversion of 4-hydroxyphenylpyruvate to homogentisate in the biosynthetic pathway (Matringe et al., 2005, Pest Manag Sci., vol. 61:269-276; Mitchell et al., 2001, Pest Manag Sci. vol 57:120-128). Plastoquinone is thought to be a necessary cofactor of the enzyme phytoene desaturase in carotenoid biosynthesis (Boeger and Sandmann, 1998, Pestic Outlook, vol 9:29-35). Its inhibition results in the depletion of the plant plastoquinone and vitamin E pools, leading to bleaching symptoms. The loss of carotenoids, particularly in their function as protectors of the photosystems against photooxidation, leads to oxidative degradation of chlorophyll and photosynthetic membranes in growing shoot tissues. Consequently, chloroplast synthesis and function are disturbed (Boeger and Sandmann, 1998). The enzyme homogentisate solanesyl transferase (HST) catalyses the step following HPPD in the plastoquinone biosynthetic pathway. HST is a prenyl transferase that both decarboxylates homogentisate and also transfers to it the solanesyl group from solanesyl diphosphate and thus forms 2-methyl-6-solanesyl-1,4-benzoquinol (MSBQ), an intermediate along the biosynthetic pathway to plastoquinone. HST enzymes are membrane bound and the genes that encode them include a plastid targeting sequence.

The most important chemical classes of commercial 4-HPPD-inhibiting herbicides include pyrazolones, triketones and isoxazoles. The inhibitors mimic the binding of the substrate 4-hydroxyphenylpyruvate to an enzyme-bound ferrous ion in the active site by forming a stable ion-dipole charge transfer complex. Among 4-HPPD-inhibiting herbicides, the triketone sulcotrione was the first example of this herbicide group to be used in agriculture and identified in its mechanism of action (Schulz et al., 1993, FEBS Lett. Vol 318:162-166) The triketones have been reported to be derivatives of leptospermone, a herbicidal component from the bottlebrush plant, Callistemon spp (Lee et al. 1997, Weed Sci. Vol 45, 162-166).

Some of these molecules have been used as herbicides since inhibition of the reaction in plants leads to whitening of the leaves of the treated plants and to the death of the said plants (Pallett, K. E. et al. 1997 Pestic. Sci. 50 83-84). The herbicides for which HPPD is the target, and which are described in the state of the art, are, in particular, isoxazoles (EP418175, EP470856, EP487352, EP527036, EP560482, EP682659, U.S. Pat. No. 5,424,276), in particular isoxaflutole, which is a selective herbicide for maize, diketonitriles (EP496630, EP496631), in particular 2-cyano-3-cyclopropyl-1-(2-SO₂CH₃-4-CF3 phenyl)propane-1,3-dione and 2-cyano-3-cyclopropyl-1-(2-SO₂CH₃-4-2,3Cl₂-phenyl)propane-1,3-dione, triketones such as described in EP625505, EP625508, U.S. Pat. No. 5,506,195, in particular sulcotrione, or else pyrazolinates. Furthermore, the well-known herbicide topramezone elicits the same type of phytotoxic symptoms, with chlorophyll loss and necrosis in the growing shoot tissues, as 4-HPPD inhibiting, bleaching herbicides described supra in susceptible plant species. Topramezone belongs to the chemical class of pyrazolones or benzoyl pyrazoles and was commercially introduced in 2006. When applied post-emergence, the compound selectively controls a wide spectrum of annual grass and broadleaf weeds in corn.

Plant tolerance to “coumarone-derivative herbicides” has also been reported in a number of patents. International application Nos. WO2010/029311 generally describes the use of an HPPD nucleic acid and/or an HST nucleic acid to elicit herbicide tolerance in plants. WO2009/090401, WO2009/090402, WO2008/071918, WO2008/009908, specifically disclose certain “coumarone-derivative herbicides” and “coumarone-derivative herbicides” tolerant plant lines.

Three main strategies are available for making plants tolerant to herbicides, i.e. (1) detoxifying the herbicide with an enzyme which transforms the herbicide, or its active metabolite, into non-toxic products, such as, for example, the enzymes for tolerance to bromoxynil or to basta (EP242236, EP337899); (2) mutating the target enzyme into a functional enzyme which is less sensitive to the herbicide, or to its active metabolite, such as, for example, the enzymes for tolerance to glyphosate (EP293356, Padgette S. R. et al., J. Biol. Chem., 266, 33, 1991); or (3) overexpressing the sensitive enzyme so as to produce quantities of the target enzyme in the plant which are sufficient in relation to the herbicide, in view of the kinetic constants of this enzyme, so as to have enough of the functional enzyme available despite the presence of its inhibitor. The third strategy was described for successfully obtaining plants which were tolerant to HPPD inhibitors (WO96/38567). US2009/0172831 discloses nucleotide sequences encoding amino acid sequences having enzymatic activity such that the amino acid sequences are resistant to HPPD inhibitor herbicidal chemicals, in particular triketone inhibitor specific HPPD mutants.

To date, the prior art has not described coumarone-derivative herbicide tolerant plants containing at least one mutated HPPD nucleic acid. Nor has the prior art described coumarone-derivative herbicide tolerant crop plants containing mutations on genomes other than the genome from which the HPPD gene is derived. Therefore, what is needed in the art is the identification of coumarone-derivative herbicide tolerance genes from additional genomes and species. What are also needed in the art are crop plants and crop plants having increased tolerance to herbicides such as coumarone-derivative herbicide and containing at least one mutated HPPD nucleic acid. Also needed are methods for controlling weed growth in the vicinity of such crop plants or crop plants. These compositions and methods would allow for the use of spray over techniques when applying herbicides to areas containing crop plants or crop plants.

### SUMMARY OF THE INVENTION

The problem is solved by the present invention which refers to a method for controlling undesired vegetation at a plant cultivation site, the method comprising the steps of:
a) providing, at said site, a plant that comprises at least one nucleic acid comprising
   (i) a nucleotide sequence encoding a wild type hydroxyphenyl pyruvate dioxygenase or a mutated hydroxyphenyl pyruvate dioxygenase (mut-HPPD) which is resistant or tolerant to a coumarone-derivative herbicide and/or
   (ii) a nucleotide sequence encoding a wildtype homogentisate solanesyl transferase or a mutated homogentisate solanesyl transferase (mut-HST) which is resistant or tolerant to a coumarone-derivative herbicide
b) applying to said site an effective amount of said herbicide.

In addition, the present invention refers to a method for identifying a coumarone-derivative herbicide by using a mut-HPPD encoded by a nucleic acid which comprises the nucleotide sequence of SEQ ID NO: 1, 3, or 5, or a variant thereof, and/or by using a mut-HST encoded by a nucleic acid which comprises the nucleotide sequence of SEQ ID NO: 7 or 9 or a variant thereof.

Said method comprises the steps of:
a) generating a transgenic cell or plant comprising a nucleic acid encoding a mut-HPPD, wherein the mut-HPPD is expressed;
b) applying a coumarone-derivative herbicide to the transgenic cell or plant of a) and to a control cell or plant of the same variety;
c) determining the growth or the viability of the transgenic cell or plant and the control cell or plant after application of said test compound, and
d) selecting test compounds which confer reduced growth to the control cell or plant as compared to the growth of the transgenic cell or plant.

Another object refers to a method of identifying a nucleotide sequence encoding a mut-HPPD which is resistant or tolerant to a coumarone-derivative herbicide, the method comprising:
a) generating a library of mut-HPPD-encoding nucleic acids,
b) screening a population of the resulting mut-HPPD-encoding nucleic acids by expressing each of said nucleic acids in a cell or plant and treating said cell or plant with a coumarone-derivative herbicide,
c) comparing the coumarone-derivative herbicide-tolerance levels provided by said population of mut-HPPD encoding nucleic acids with the coumarone-derivative herbicide-tolerance level provided by a control HPPD-encoding nucleic acid,
d) selecting at least one mut-HPPD-encoding nucleic acid that provides a significantly increased level of tolerance to a coumarone-derivative herbicide as compared to that provided by the control HPPD-encoding nucleic acid.

In a preferred embodiment, the mut-HPPD-encoding nucleic acid selected in step d) provides at least 2-fold as much or tolerance to a coumarone-derivative herbicide as compared to that provided by the control HPPD-encoding nucleic acid.

The resistance or tolerance can be determined by generating a transgenic plant comprising a nucleic acid sequence of the library of step a) and comparing said transgenic plant with a control plant.

Another object refers to a method of identifying a plant or algae containing a nucleic acid encoding a mut-HPPD or mut-HST which is resistant or tolerant to a coumarone-derivative herbicide, the method comprising:
a) identifying an effective amount of a coumarone-derivative herbicide in a culture of plant cells or green algae.
b) treating said plant cells or green algae with a mutagenizing agent,
c) contacting said mutagenized cells population with an effective amount of coumarone-derivative herbicide, identified in a),
d) selecting at least one cell surviving these test conditions,
e) PCR-amplification and sequencing of HPPD and/or HST genes from cells selected in d) and comparing such sequences to wild-type HPPD or HST gene sequences, respectively.

In a preferred embodiment, the mutagenizing agent is ethylmethanesulfonate.

Another object refers to an isolated nucleic acid encoding a mut-HPPD, the nucleic acid being identifiable by a method as defined above.

In another embodiment, the invention refers to a plant cell transformed by a wild-type or a mut-HPPD nucleic acid or or a plant which has been mutated to obtain a plant expressing, preferably over-expressing, a wild-type or a mut-HPPD nucleic acid, wherein expression of the nucleic acid in the plant cell results in increased resistance or tolerance to a coumarone-derivative herbicide as compared to a wild type variety of the plant cell.

In another embodiment, the invention refers to a transgenic plant comprising a plant cell according to the present invention, wherein expression of the nucleic acid in the plant results in the plant's increased resistance to coumarone-derivative herbicide as compared to a wild type variety of the plant.

The plants of the present invention can be transgenic or non-transgenic.

Preferably, the expression of the nucleic acid in the plant results in the plant's increased resistance to coumarone-derivative herbicide as compared to a wild type variety of the plant.

In another embodiment, the invention refers to a seed produced by a transgenic plant comprising a plant cell of the present invention, wherein the seed is true breeding for an increased resistance to a coumarone-derivative herbicide as compared to a wild type variety of the seed.

In another embodiment, the invention refers to a method of producing a transgenic plant cell with an increased resistance to a coumarone-derivative herbicide as compared to a wild type variety of the plant cell comprising, transforming the plant cell with an expression cassette comprising a wild-type or a mut-HPPD nucleic acid.

In another embodiment, the invention refers to a method of producing a transgenic plant comprising, (a) transforming a plant cell with an expression cassette comprising a wild-type or a mut-HPPD nucleic acid, and (b) generating a plant with an increased resistance to coumarone-derivative herbicide from the plant cell.

Preferably, the expression cassette further comprises a transcription initiation regulatory region and a translation initiation regulatory region that are functional in the plant.

In another embodiment, the invention relates to using the mut-HPPD of the invention as selectable marker. The invention provides a method of identifying or selecting a transformed plant cell, plant tissue, plant or part thereof comprising a) providing a transformed plant cell, plant tissue, plant or part thereof, wherein said transformed plant cell, plant tissue, plant or part thereof comprises an isolated nucleic acid encoding a mut-HPPD polypeptide of the invention as described hereinafter, wherein the polypeptide is used as a selection marker, and wherein said transformed plant cell, plant tissue, plant or part thereof may optionally comprise a further isolated nucleic acid of interest; b) contacting the transformed plant cell, plant tissue, plant or part thereof with at least one coumarone-derivative inhibiting compound; c) determining whether the plant cell, plant tissue, plant or part thereof is affected by the inhibitor or inhibiting compound; and d) identifying or selecting the transformed plant cell, plant tissue, plant or part thereof.

The invention is also embodied in purified mut-HPPD proteins that contain the mutations described herein, which are useful in molecular modeling studies to design further improvements to herbicide tolerance. Methods of protein purification are well known, and can be readily accomplished using commercially available products or specially designed methods, as set forth for example, in Protein Biotechnology, Walsh and Headon (Wiley, 1994).

### BRIEF DESCRIPTION OF THE DRAWINGS

FIG. 1 Amino acid sequence alignment and conserved regions of HPPD enzymes from Chlamydomonas reinhardtii (Cr_HPPD1a, Cr_HPPD1b), Physcomitrella patens (Pp_HPPD1), Oryza sativa (Osj_HPPD1), Triticum aestivum (Ta_HPPD1), Zea mays (Zm_HPPD1), Arabidopsis thaliana (At_HPPD), Glycine max (Gm_HPPD) and Vitis vinifera (Vv_HPPD).

* Sequence derived from genome sequencing project. Locus ID: GRMZM2G088396
** Amino acid sequence based on NCBI GenPept accession CAG25475

FIG. 2 Selection of Chlamydomonas reinhardtii strains resistant to “coumarone-derivative herbicides”. (A) Mutagenized cells plated on solid medium without a selecting agent. (B) Mutagenized cells plated on solid medium containing 50 μM 4-hydroxy-3-[2-methyl-3-(5-methyl-4,5-dihydro-isoxazol-3-yl)-4-methylsulfonyl-phenyl]pyrano[3,2-b]pyridin-2-one. Cells which are resistant to “coumarone-derivative herbicides” are able to form colonies (circled), while susceptible cells are not able to grow.

FIG. 3 shows a vector map of a plant transformation vector which is used for soybean transformation with HPPD/HST sequences.

FIG. 4 Herbicide spray tests against transgenic T0 soybean cuttings expressing Arabidopsis wild type HPPD (AtHPPD). AV3639, AV3641 and AV3653 are individual events. Non-transformed control plants are marked as wild type. The “coumarone-derivative” marked with an asterisk corresponds to * 3-[2,4-dichloro-3-(3-methyl-4,5-dihydro-isoxazol-5-yl)phenyl]-1-(2,2-difluoroethyl)-2,2-dioxo-pyrido[3,2-c]thiazin-4-ol.

### SEQUENCE LISTING

TABLE 1

| SEQ ID NO: | Description | Organism | Locus | Accession number |
|---|---|---|---|---|
| 1 | HPPD nucleic acid | Arabidopsis | At1g06570 | AF047834 |
| 2 | HPPD amino acid | Arabidopsis | At1g06570 | AAC15697 |
| 3 | HPPD nucleic acid1 | Chlamydomonas | | |
| 4 | HPPD amino acid1 | Chlamydomonas | | |
| 5 | HPPD nucleic acid2 | Chlamydomonas | | XM_001694671.1 |
| 6 | HPPD amino acid2 | Chlamydomonas | | Q70ZL8 |
| 7 | HST nucleic acid | Arabidopsis | At3g11945 | DQ231060 |
| 8 | HST amino acid | Arabidopsis | At3g11945 | Q1ACB3 |
| 9 | HST nucleic acid | Chlamydomonas | | AM285678 |
| 10 | HST amino acid | Chlamydomonas | | A1JHN0 |
| 11 | HPPD amino acid | Physcomitrella | | A9RPY0 |
| 12 | HPPD amino acid | Oryza | Os02g07160 | |
| 13 | HPPD amino acid | Triticum | | Q45FE8 |
| 14 | HPPD amino acid | Zea | | CAG25475 |
| 15 | HPPD amino acid | Glycine | | A5Z1N7 |
| 16 | HPPD amino acid | Vitis | | A5ADC8 |
| 17 | HPPD amino acid | Pseudomonas fluorescens | | AXW96633 |
| | | strain 87-79 | | |
| 18 | HPPD amino acid | Pseudomonas fluorescens | | ADR00548 |
| 19 | HPPD amino acid | Avena sativa | | AXW96634 |

### DETAILED DESCRIPTION

The articles “a” and “an” are used herein to refer to one or more than one (i.e., to at least one) of the grammatical object of the article. By way of example, “an element” means one or more elements.

As used herein, the word “comprising,” or variations such as “comprises” or “comprising,” will be understood to imply the inclusion of a stated element, integer or step, or group of elements, integers or steps, but not the exclusion of any other element, integer or step, or group of elements, integers or steps.

The present invention refers to a method for controlling undesired vegetation at a plant cultivation site, the method comprising the steps of:
c) providing, at said site, a plant that comprises at least one nucleic acid comprising
   (i) a nucleotide sequence encoding a wild-type hydroxyphenyl pyruvate dioxygenase (HPPD) or a mutated hydroxyphenyl pyruvate dioxygenase (mut-HPPD) which is resistant or tolerant to a “coumarone-derivative herbicide” and/or
   (ii) a nucleotide sequence encoding a wild-type homogentisate solanesyl transferase (HST) or a mutated homogentisate solanesyl transferase (mut-HST) which is resistant or tolerant to a “coumarone-derivative herbicide”
d) applying to said site an effective amount of said herbicide.

The term “control of undesired vegetation” is to be understood as meaning the killing of weeds and/or otherwise retarding or inhibiting the normal growth of the weeds. Weeds, in the broadest sense, are understood as meaning all those plants which grow in locations where they are undesired. The weeds of the present invention include, for example, dicotyledonous and monocotyledonous weeds. Dicotyledonous weeds include, but are not limited to, weeds of the genera: Sinapis, Lepidium, Galium, Stellaria, Matricaria, Anthemis, Galinsoga, Chenopodium, Urtica, Senecio, Amaranthus, Portulaca, Xanthium, Convolvulus, Ipomoea, Polygonum, Sesbania, Ambrosia, Cirsium, Carduus, Sonchus, Solanum, Rorippa, Rotala, Lindernia, Lamium, Veronica, Abutilon, Emex, Datura, Viola, Galeopsis, Papaver, Centaurea, Trifolium, Ranunculus, and Taraxacum. Monocotyledonous weeds include, but are not limited to, weeds of the genera: Echinochloa, Setaria, Panicum, Digitaria, Phleum, Poa, Festuca, Eleusine, Brachiaria, Lolium, Bromus, Avena, Cyperus, Sorghum, Agropyron, Cynodon, Monochoria, Fimbristyslis, Sagittaria, Eleocharis, Scirpus, Paspalum, Ischaemum, Sphenoclea, Dactyloctenium, Agrostis, Alopecurus, and Apera. In addition, the weeds of the present invention can include, for example, crop plants that are growing in an undesired location. For example, a volunteer maize plant that is in a field that predominantly comprises soybean plants can be considered a weed, if the maize plant is undesired in the field of soybean plants.

The term “plant” is used in its broadest sense as it pertains to organic material and is intended to encompass eukaryotic organisms that are members of the Kingdom Plantae, examples of which include but are not limited to vascular plants, vegetables, grains, flowers, trees, herbs, bushes, grasses, vines, ferns, mosses, fungi and algae, etc, as well as clones, offsets, and parts of plants used for asexual propagation (e.g. cuttings, pipings, shoots, rhizomes, underground stems, clumps, crowns, bulbs, corms, tubers, rhizomes, plants/tissues produced in tissue culture, etc.). The term “plant” further encompasses whole plants, ancestors and progeny of the plants and plant parts, including seeds, shoots, stems, leaves, roots (including tubers), flowers, florets, fruits, pedicles, peduncles, stamen, anther, stigma, style, ovary, petal, sepal, carpel, root tip, root cap, root hair, leaf hair, seed hair, pollen grain, microspore, cotyledon, hypocotyl, epicotyl, xylem, phloem, parenchyma, endosperm, a companion cell, a guard cell, and any other known organs, tissues, and cells of a plant, and tissues and organs, wherein each of the aforementioned comprise the gene/nucleic acid of interest. The term “plant” also encompasses plant cells, suspension cultures, callus tissue, embryos, meristematic regions, gametophytes, sporophytes, pollen and microspores, again wherein each of the aforementioned comprises the gene/nucleic acid of interest.

Plants that are particularly useful in the methods of the invention include all plants which belong to the superfamily Viridiplantae, in particular monocotyledonous and dicotyledonous plants including fodder or forage legumes, ornamental plants, food crops, trees or shrubs selected from the list comprising Acer spp., Actinidia spp., Abelmoschus spp., Agave sisalana, Agropyron spp., Agrostis stolonifera, Allium spp., Amaranthus spp., Ammophila arenaria, Ananas comosus, Annona spp., Apium graveolens, Arachis spp, Artocarpus spp., Asparagus officinalis, Avena spp. (e.g. Avena sativa, Avena fatua, Avena byzantina, Avena fatua var. sativa, Avena hybrida), Averrhoa carambola, Bambusa sp., Benincasa hispida, Bertholletia excelsea, Beta vulgaris, Brassica spp. (e.g. Brassica napus, Brassica rapa ssp. [canola, oilseed rape, turnip rape]), Cadaba farinosa, Camellia sinensis, Canna indica, Cannabis sativa, Capsicum spp., Carex elata, Carica papaya, Carissa macrocarpa, Carya spp., Carthamus tinctorius, Castanea spp., Ceiba pentandra, Cichorium endivia, Cinnamomum spp., Citrullus lanatus, Citrus spp., Cocos spp., Coffea spp., Colocasia esculenta, Cola spp., Corchorus sp., Coriandrum sativum, Corylus spp., Crataegus spp., Crocus sativus, Cucurbita spp., Cucumis spp., Cynara spp., Daucus carota, Desmodium spp., Dimocarpus longan, Dioscorea spp., Diospyros spp., Echinochloa spp., Elaeis (e.g. Elaeis guineensis, Elaeis oleifera), Eleusine coracana, Eragrostis tef, Erianthus sp., Eriobotrya japonica, Eucalyptus sp., Eugenia uniflora, Fagopyrum spp., Fagus spp., Festuca arundinacea, Ficus carica, Fortunella spp., Fragaria spp., Ginkgo biloba, Glycine spp. (e.g. Glycine max, Soja hispida or Soja max), Gossypium hirsutum, Helianthus spp. (e.g. Helianthus annuus), Hemerocaffis fulva, Hibiscus spp., Hordeum spp. (e.g. Hordeum vulgare), Ipomoea batatas, Juglans spp., Lactuca sativa, Lathyrus spp., Lens culinaris, Linum usitatissimum, Litchi chinensis, Lotus spp., Luffa acutangula, Lupinus spp., Luzula sylvatica, Lycopersicon spp. (e.g. Lycopersicon esculentum, Lycopersicon lycopersicum, Lycopersicon pyriforme), Macrotyloma spp., Malus spp., Malpighia emarginata, Mammea americana, Mangifera indica, Manihot spp., Manilkara zapota, Medicago sativa, Melilotus spp., Mentha spp., Miscanthus sinensis, Momordica spp., Morus nigra, Musa spp., Nicotiana spp., Olea spp., Opuntia spp., Ornithopus spp., Oryza spp. (e.g. Oryza sativa, Oryza latifolia), Panicum miliaceum, Panicum virgatum, Passiflora edulis, Pastinaca sativa, Pennisetum sp., Persea spp., Petroselinum crispum, Phalaris arundinacea, Phaseolus spp., Phleum pratense, Phoenix spp., Phragmites australis, Physalis spp., Pinus spp., Pistacia vera, Pisum spp., Poa spp., Populus spp., Prosopis spp., Prunus spp., Psidium spp., Punica granatum, Pyrus communis, Quercus spp., Raphanus sativus, Rheum rhabarbarum, Ribes spp., Ricinus communis, Rubus spp., Saccharum spp., Salix sp., Sambucus spp., Secale cereale, Sesamum spp., Sinapis sp., Solanum spp. (e.g. Solanum tuberosum, Solanum integrifolium or Solanum lycopersicum), Sorghum bicolor, Spinacia spp., Syzygium spp., Tagetes spp., Tamarindus indica, Theobroma cacao, Trifolium spp., Tripsacum dactyloides, Triticosecale rimpaui, Triticum spp. (e.g. Triticum aestivum, Triticum durum, Triticum turgidum, Triticum hybernum, Triticum macha, Triticum sativum, Triticum monococcum or Triticum vulgare), Tropaeolum minus, Tropaeolum majus, Vaccinium spp., Vicia spp., Vigna spp., Viola odorata, Vitis spp., Zea mays, Zizania palustris, Ziziphus spp., amaranth, artichoke, asparagus, broccoli, Brussels sprouts, cabbage, canola, carrot, cauliflower, celery, collard greens, flax, kale, lentil, oilseed rape, okra, onion, potato, rice, soybean, strawberry, sugar beet, sugar cane, sunflower, tomato, squash, tea and algae, amongst others. According to a preferred embodiment of the present invention, the plant is a crop plant. Examples of crop plants include inter alia soybean, sunflower, canola, alfalfa, rapeseed, cotton, tomato, potato or tobacco. Further preferebly, the plant is a monocotyledonous plant, such as sugarcane. Further preferably, the plant is a cereal, such as rice, maize, wheat, barley, millet, rye, sorghum or oats.

In a preferred embodiment, the plant has been previously produced by a process comprising recombinantly preparing a plant by introducing and over-expressing a wild-type or mut-HPPD and/or wild-type or mut-HST transgene, as described in greater detail hereinfter.

In another preferred embodiment, the plant has been previously produced by a process comprising in situ mutagenizing plant cells, to obtain plant cells which express a mut-HPPD and/or mut-HST.

As disclosed herein, the nucleic acids of the invention find use in enhancing the herbicide tolerance of plants that comprise in their genomes a gene encoding a herbicide-tolerant wild-type or mut-HPPD and/or wild-type or mut-HST protein. Such a gene may be an endogenous gene or a transgene, as described hereinafter. Additionally, in certain embodiments, the nucleic acids of the present invention can be stacked with any combination of polynucleotide sequences of interest in order to create plants with a desired phenotype. For example, the nucleic acids of the present invention may be stacked with any other polynucleotides encoding polypeptides having pesticidal and/or insecticidal activity, such as, for example, the Bacillus thuringiensis toxin proteins (described in U.S. Pat. Nos. 5,366,892; 5,747,450; 5,737,514; 5,723,756; 5,593,881; and Geiser et al (1986) Gene 48: 109). The combinations generated can also include multiple copies of any one of the polynucleotides of interest.

In a particularly preferred embodiment, the plant comprises at least one additional heterologous nucleic acid comprising (iii) a nucleotide sequence encoding a herbicide tolerance enzyme selected, for example, from the group consisting of 5-enolpyruvylshikimate-3-phosphate synthase (EPSPS), Glyphosate acetyl transferase (GAT), Cytochrome P450, phosphinothricin acetyltransferase (PAT), Acetohydroxyacid synthase (AHAS; EC 4.1.3.18, also known as acetolactate synthase or ALS), Protoporphyrinogen oxidase (PPGO), Phytoene desaturase (PD) and dicamba degrading enzymes as disclosed in WO 02/068607.

Generally, the term “herbicide” is used herein to mean an active ingredient that kills, controls or otherwise adversely modifies the growth of plants. The preferred amount or concentration of the herbicide is an “effective amount” or “effective concentration.” By “effective amount” and “effective concentration” is intended an amount and concentration, respectively, that is sufficient to kill or inhibit the growth of a similar, wild-type, plant, plant tissue, plant cell, or host cell, but that said amount does not kill or inhibit as severely the growth of the herbicide-resistant plants, plant tissues, plant cells, and host cells of the present invention. Typically, the effective amount of a herbicide is an amount that is routinely used in agricultural production systems to kill weeds of interest. Such an amount is known to those of ordinary skill in the art. Herbicidal activity is exhibited by coumarone-derivative herbicide of the present invention when they are applied directly to the plant or to the locus of the plant at any stage of growth or before planting or emergence. The effect observed depends upon the plant species to be controlled, the stage of growth of the plant, the application parameters of dilution and spray drop size, the particle size of solid components, the environmental conditions at the time of use, the specific compound employed, the specific adjuvants and carriers employed, the soil type, and the like, as well as the amount of chemical applied. These and other factors can be adjusted as is known in the art to promote non-selective or selective herbicidal action. Generally, it is preferred to apply the coumarone-derivative herbicide postemergence to relatively immature undesirable vegetation to achieve the maximum control of weeds.

By a “herbicide-tolerant” or “herbicide-resistant” plant, it is intended that a plant that is tolerant or resistant to at least one herbicide at a level that would normally kill, or inhibit the growth of, a normal or wild-type plant. By “herbicide-tolerant mut-HPPD protein” or “herbicide-resistant mut-HPPD protein”, it is intended that such a mut-HPPD protein displays higher HPPD activity, relative to the HPPD activity of a wild-type mut-HPPD protein, when in the presence of at least one herbicide that is known to interfere with HPPD activity and at a concentration or level of the herbicide that is known to inhibit the HPPD activity of the wild-type mut-HPPD protein. Furthermore, the HPPD activity of such a herbicide-tolerant or herbicide-resistant mut-HPPD protein may be referred to herein as “herbicide-tolerant” or “herbicideresistant” HPPD activity.

The “coumarone-derivative herbicide” of the present invention encompasses the compounds as depicted in the following Table 2.

TABLE 2

| | | Possible Substituents as defined in: | | |
|---|---|---|---|---|
| | | Application number | Publication | |
| No: | General Structure | and reference | Number | Pages |
| 1 | 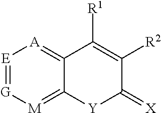 I | PCT/EP2009/063387 (PF61381-1) | WO2010/049270 | 1 to 2 |
| 2 | 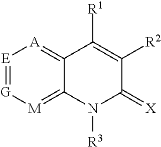 I | PCT/EP2009/063386 (PF61381-2) | WO2010/049269 | 1 to 2 |
| 3 | 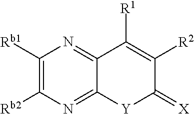 I | EP09162085.6 (PF62203) | WO2010/139657 WO2010/139658 | 1 to 2 |
| 4 | 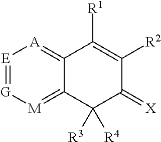 I | EP09174833.5 EP10189606.6 (PF62704) | | 1 to 2 |
| 5 | 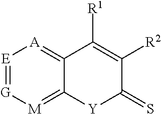 I | EP09174585.1 (PF62698) | | 1 to 2 |
| 6 | 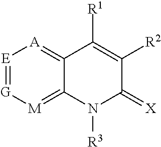 I | EP09175673.4 PCT/EP2010/067059 (PF62736) | | 1 to 2 |
| 7 | 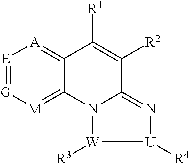 I | EP09175959.7 PCT/EP2010/067176 (PF62752) | | 1 to 2 |
| 8 | 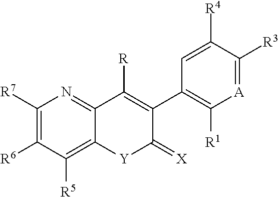 I | EP10157312.9 US61/316400 PCT/EP2011/054258 (PF70482) | | 1 to 2 |
| 9 | 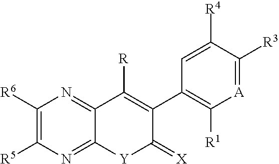 I | EP10157290.7 US61/316394 PCT/EP2011/054281 (PF70483) | | 1 to 3 |
| 10 | 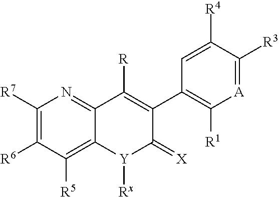 I | EP10157296.4 US61/316398 PCT/EP2011/054280 (PF70484) | | 1 to 3 |
| 11 | 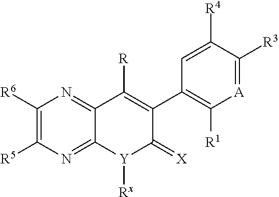 I | EP10157282.4 US61/316396 PCT/EP2011/054403 (PF70485) | | 1 to 3 |
| 12 | 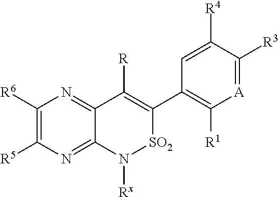 I | EP10157352.5 US61/316405 PCT/EP2011/054128 PF70528 | | 1 to 3 |
| 13 | 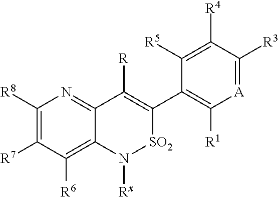 I | EP10157419.2 US61/316461 PCT/EP2011/054129 (PF70527) | | 1 to 2 |
| 14 | Formulas | PCT/GB2009/002188 | WO2010/029311 | 3 to 11; |
| | Ia, Ib, Ic, Id, Ie, If, | | | 12 to |
| | Iia, Iib, Iic, Iid, Iie, Iif | | | 18 |
| 15 | Formula I (a to d) | PCT/GB2009/000126 | WO2009/090401 | 1 to 17 |
| 16 | Formula I (a to d) | PCT/GB2009/000127 | WO2009/090402 | 1 to 17 |
| 17 | Formula I (a, d) | PCT/GB2007/004662 | WO2008/071918 | 1 to 11 |
| 18 | Formula I (a to d) | PCT/GB2007/002668 | WO2008/009908 | 1 to 16 |

The above referenced applications, in particular the disclosures referring to the compounds of Table 2 and their possible substitutents are entirely incorporated by reference.

A particular preferred embodiment of the present invention refers to a coumarone derivative herbicide of Number 13 of Table 2 above having the formula:

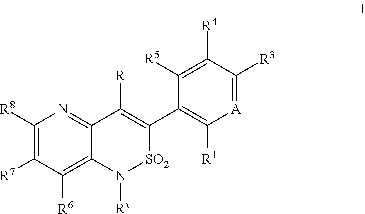

in which the variables have the following meaning:
R is O-RA, S(O)ₙ—R^{A} or O—S(O)ₙ—RA;
   R^{A} is hydrogen, C₁-C₄-alkyl, Z—C₃-C₆-cycloalkyl, C₁-C₄-haloalkyl, C₂-C₆-alkenyl, Z—C₃-C₆-cycloalkenyl, C₂-C₆-alkynyl, Z-(tri-C₁-C₄-alkyl)silyl, Z—C(═O)—Ra, Z—NRⁱ—C(O)—NRⁱRⁱⁱ, Z—P(═O)(R^{a})₂, NRⁱRⁱⁱ, a 3- to 7-membered monocyclic or 9- or 10-membered bicyclic saturated, unsaturated or aromatic heterocycle which contains 1, 2, 3 or 4 heteroatoms selected from the group consisting of O, N and S and which may be partially or fully substituted by groups R^{a} and/or R^{b},
      R^{a} is hydrogen, OH, C₁-C₈-alkyl, C₁-C₄-haloalkyl, Z—C₃-C₆-cycloalkyl, C₂-C₈-alkenyl, Z—C₅-C₆-cycloalkenyl, C₂-C₈-alkynyl, Z—C₁-C₆-alkoxy, Z—C₁-C₄-haloalkoxy, Z—C₃-C₈-alkenyloxy, Z—C₃-C₈-alkynyloxy, NRⁱRⁱⁱ, C₁-C₆-alkylsulfonyl, Z-(tri-C₁-C₄-alkyl)silyl, Z-phenyl, Z-phenoxy, Z-phenylamino or a 5- or 6-membered monocyclic or 9- or 10-membered bicyclic heterocycle which contains 1, 2, 3 or 4 heteroatoms selected from the group consisting of O, N and S, where the cyclic groups are unsubstituted or substituted by 1, 2, 3 or 4 groups Rb;
         Rⁱ, Rⁱⁱ independently of one another are hydrogen, C₁-C₈-alkyl, C₁-C₄-haloalkyl, C₃-C₈-alkenyl, C₃-C₈-alkynyl, Z—C₃-C₆-cycloalkyl, Z—C₁-C₈-alkoxy, Z—C₁-C₈-haloalkoxy, Z—C(═O)—R^{a}, Z-phenyl, a 3- to 7-membered monocyclic or 9- or 10-membered bicyclic saturated, unsaturated or aromatic heterocycle which contains 1, 2, 3 or 4 heteroatoms selected from the group consisting of O, N and S and which is attached via Z;
         Rⁱ and Rⁱⁱ together with the nitrogen atom to which they are attached may also form a 5- or 6-membered monocyclic or 9- or 10-membered bicyclic heterocycle which contains 1, 2, 3 or 4 heteroatoms selected from the group consisting of O, N and S;
      R^{b} independently of one another are Z—CN, Z—OH, Z—NO₂, Z-halogen, oxo (═O), ═N—R^{a}, C₁-C₈-alkyl, C₁-C₄-haloalkyl, C₂-C₈-alkenyl, C₂-C₈-alkynyl, Z—C₁-C₈-alkoxy, Z—C₁-C₈-haloalkoxy, Z—C₃-C₁₀-cycloalkyl, O—Z—C₃-C₁₀-cycloalkyl, Z—C(═O)—Ra, NRⁱRⁱⁱ, Z-(tri-C₁-C₄-alkyl)silyl, Z-phenyl and S(O)ₙR^{bb}; two groups R^{b} may together form a ring which has three to six ring members and, in addition to carbon atoms, may also contain heteroatoms from the group consisting of O, N and S and may be unsubstituted or substituted by further groups Rb;
         R^{bb} is C₁-C₈-alkyl, C₂-C₆-alkenyl, C₂-C₆-alkynyl, C₂-C₆-haloalkenyl, C₂-C₆-haloalkynyl or C₁-C₆-haloalkyl;
      Z is a covalent bond or C₁-C₄-alkylene;
   n is 0, 1 or 2;
R¹ is cyano, halogen, nitro, C₁-C₆-alkyl, C₂-C₆-alkenyl, C₂-C₆-alkynyl, C₁-C₆-haloalkyl, Z—C₁-C₆-alkoxy, Z—C₁-C₄-alkoxy-C₁-C₄-alkoxy, Z—C₁-C₄-alkylthio, Z—C₁-C₄-alkylthio-C₁-C₄-alkylthio, C₂-C₆-alkenyloxy, C₂-C₆-alkynyloxy, C₁-C₆-haloalkoxy, C₁-C₄-haloalkoxy-C₁-C₄-alkoxy, S(O)ₙR^{bb}, Z-phenoxy, Z-heterocyclyloxy, where heterocyclyl is a 5- or 6-membered monocyclic or 9- or 10-membered bicyclic saturated, partially unsaturated or aromatic heterocycle which contains 1, 2, 3 or 4 heteroatoms selected from the group consisting of O, N and S, where cyclic groups are unsubstituted or partially or fully substituted by R^{b};
A is N or O—R²;
R², R³, R⁴, R⁵ independently of one another are hydrogen, Z-halogen, Z—CN, Z—OH, Z—NO₂, C₁-C₈-alkyl, C₁-C₄-haloalkyl, C₂-C₈-alkenyl, C₂-C₈-alkynyl, C₂-C₈-haloalkenyl, C₂-C₈-haloalkynyl, ZC₁-C₈-alkoxy, Z—C₁-C₈-haloalkoxy, Z—C₁-C₄-alkoxy-C₁-C₄-alkoxy, Z—C₁-C₄-alkylhio, Z—C₁-C₄-alkylthio-C₁-C₄-alkylthio, Z—C₁-C₆-haloalkylthio, C₂-C₆-alkenyloxy, C₂-C₆-alkynyloxy, C₁-C₆-haloalkoxy, C₁-C₄-haloalkoxy-C₁-C₄-alkoxy, Z—C₃-C₁₀-cycloalkyl, O—Z—C₃-C₁₀-cycloalkyl, ZC(═O)—Ra, NRⁱRⁱⁱ, Z-(tri-C₁-C₄-alkyl)silyl, S(O)ₙR^{bb}, Z-phenyl, Z¹-phenyl, Z-heterocyclyl, Z¹-heterocyclyl, where heterocyclyl is a 5- or 6-membered monocyclic or 9- or 10-membered bicyclic saturated, partially unsaturated or aromatic heterocycle which contains 1, 2, 3 or 4 heteroatoms selected from the group consisting of O, N and S, where cyclic groups are unsubstituted or partially or fully substituted by R^{b};
   R² together with the group attached to the adjacent carbon atom may also form a five- to ten-membered saturated or partially or fully unsaturated mono- or bicyclic ring which, in addition to carbon atoms, may contain 1, 2 or 3 heteroatoms selected from the group consisting of O, N and S and may be substituted by further groups R^{b};
   Z¹ is a covalent bond, C₁-C₄-alkyleneoxy, C₁-C₄-oxyalkylene or C₁-C₄-alkyleneoxy-C₁-C₄-alkylene;
R⁶ is hydrogen, C₁-C₄-alkyl, C₁-C₄-haloalkyl, C₁-C₄-alkoxy, C₁-C₄-alkylthio, C₁-C₄-haloalkoxy, C₁-C₄-haloalkylthio;
R⁷, R⁸ independently of one another are hydrogen, halogen or C₁-C₄-alkyl;
R^{x} is C₁-C₆-alkyl, C₁-C₄-haloalkyl, C₁-C₂-alkoxy-C₁-C₂-alkyl, C₂-C₆-alkenyl, C₂-C₆-haloalkenyl, C₃-C₆-alkynyl, C₃-C₆-haloalkynyl or Z-phenyl, which is unsubstituted or substituted by 1 to 5 groups R^{b};
   where in the groups R^{A}, and R¹, R², R³, R⁴ and R⁵ and their subsubstituents, the carbon chains and/or the cyclic groups may be partially or fully substituted by groups R^{b},
   or an N-oxide or an agriculturally suitable salt thereof.

A further preferred embodiment of the present invention refers to a coumarone derivative herbicide of Numbers 1 and 2 of Table 2 above having the formula:

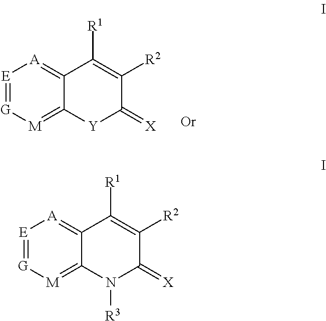

in which the variables are as disclosed in WO2010/049270 and WO2010/049269.

In a further preferred embodiment, the coumarine derivative herbicide useful for the present invention has the following formula (Table 2, No. 8)

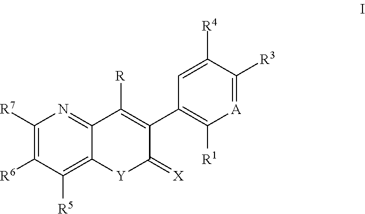

in which the variables have the following meaning:
R is O-RA, S(O)ₙ—R^{A} or O—S(O)ₙ—R^{A};
   R^{A} is hydrogen, C₁-C₄-alkyl, Z—C₃-C₆-cycloalkyl, C₁-C₄-haloalkyl, C₂-C₆-alkenyl, Z—C₃-C₆-cycloalkenyl, C₂-C₆-alkynyl, Z-(tri-C₁-C₄-alkyl)silyl, Z—C(═O)—Ra, Z—NRⁱ—C(O)—NRⁱRⁱⁱ, Z—P(═O)(R^{a})₂, NRⁱRⁱⁱ, a 3- to 7-membered monocyclic or 9- or 10-membered bicyclic saturated, unsaturated or aromatic heterocycle which contains 1, 2, 3 or 4 heteroatoms selected from the group consisting of O, N and S and which may be partially or fully substituted by groups R^{a} and/or R^{b},
      R^{a} is hydrogen, OH, C₁-C₈-alkyl, C₁-C₄-haloalkyl, Z—C₃-C₆-cycloalkyl, C₂-C₈-alkenyl, Z—C₅-C₆-cycloalkenyl, C₂-C₈-alkynyl, Z—C₁-C₆-alkoxy, Z—C₁-C₄-haloalkoxy, Z—C₃-C₈-alkenyloxy, Z—C₃-C₈-alkynyloxy, NRⁱRⁱⁱ, C₁-C₆-alkylsulfonyl, Z-(tri-C₁-C₄-alkyl)silyl, Z-phenyl, Z-phenoxy, Z-phenylamino or a 5- or 6-membered monocyclic or 9- or 10-membered bicyclic heterocycle which contains 1, 2, 3 or 4 heteroatoms selected from the group consisting of O, N and S, where the cyclic groups are unsubstituted or substituted by 1, 2, 3 or 4 groups R^{b};
         Rⁱ, Rⁱⁱ independently of one another are hydrogen, C₁-C₈-alkyl, C₁-C₄-haloalkyl, C₃-C₈-alkenyl, C₃-C₈-alkynyl, Z—C₃-C₆-cycloalkyl, Z—C₁-C₈-alkoxy, Z—C₁-C₈-haloalkoxy, Z—C(═O)—R^{a}, Z-phenyl, a 3- to 7-membered monocyclic or 9- or 10-membered bicyclic saturated, unsaturated or aromatic heterocycle which contains 1, 2, 3 or 4 heteroatoms selected from the group consisting of O, N and S and which is attached via Z;
         Rⁱ and Rⁱⁱ together with the nitrogen atom to which they are attached may also form a 5- or 6-membered monocyclic or 9- or 10-membered bicyclic heterocycle which contains 1, 2, 3 or 4 heteroatoms selected from the group consisting of O, N and S;
      Z is a covalent bond or C₁-C₄-alkylene;
   n is 0, 1 or 2;
R¹ is cyano, halogen, nitro, C₁-C₆-alkyl, C₂-C₆-alkenyl, C₂-C₆-alkynyl, C₁-C₆-haloalkyl, Z—C₁-C₆-alkoxy, Z—C₁-C₄-alkoxy-C₁-C₄-alkoxy, Z—C₁-C₄-alkylthio, Z—C₁-C₄-alkylthio-C₁-C₄-alkylthio, C₂-C₆-alkenyloxy, C₂-C₆-alkynyloxy, C₁-C₆-haloalkoxy, C₁-C₄-haloalkoxy-C₁-C₄-alkoxy, S(O)ₙR^{bb}, Z-phenoxy, Z-heterocyclyloxy, where heterocyclyl is a 5- or 6-membered monocyclic or 9- or 10-membered bicyclic saturated, partially unsaturated or aromatic heterocycle which contains 1, 2, 3 or 4 heteroatoms selected from the group consisting of O, N and S, where cyclic groups are unsubstituted or partially or fully substituted by R^{b};
   R^{bb} is C₁-C₈-alkyl, C₂-C₆-alkenyl, C₂-C₆-alkynyl, C₂-C₆-haloalkenyl, C₂-C₆-haloalkynyl or C₁-C₆-haloalkyl and n is 0, 1 or 2;
A is N or C—R²;
R² is Z¹-phenyl, phenoxy or Z¹-heterocyclyl, where heterocyclyl is a 5- or 6-membered monocyclic or 9- or 10-membered bicyclic saturated, partially unsaturated or aromatic heterocycle which contains 1, 2, 3 or 4 heteroatoms selected from the group consisting of O, N and S, where cyclic groups are unsubstituted or partially or fully substituted by R^{b};
   C₁-C₈-alkyl, C₂-C₄-haloalkyl, C₁-C₄-alkoxy-C₁-C₄-alkyl, C₁-C₄-alkylthio-C₁-C₄-alkyl, C₂-C₈-alkenyl, C₂-C₈-alkynyl, C₂-C₈-haloalkenyl, C₂-C₈-haloalkynyl, C₂-C₆-alkoxy, Z—C₁-C₄-alkoxyC₁-C₄-alkoxy, Z—C₁-C₄-haloalkoxy-C₁-C₄-alkoxy, C₂-C₆-haloalkoxy, C₃-C₆-alkenyloxy, C₃-C₆-alkynyloxy, C₂-C₆-alkylthio, C₂-C₆-haloalkylthio, Z—C(═O)—Ra, S(O)₁₋₂R^{bb};
   Z¹ is a covalent bond, C₁-C₄-alkyleneoxy, C₁-C₄-oxyalkylene or C₁-C₄-alkyleneoxy-C₁-C₄-alkylene;
   R^{b} independently of one another are Z—CN, Z—OH, Z—NO₂, Z-halogen, oxo (═O), ═N—R^{a}, C₁-C₈-alkyl, C₁-C₄-haloalkyl, C₂-C₈-alkenyl, C₂-C₈-alkynyl, Z—C₁-C₈-alkoxy, Z—C₁-C₈-haloalkoxy, Z—C₃-C₁₀-cycloalkyl, O—Z—C₃-C₁₀-cycloalkyl, Z—C(═O)—R^{a}, NRⁱRⁱⁱ, Z-(tri-C₁-C₄-alkyl)silyl, Z-phenyl and S(O)ₙR^{bb}, two groups R^{b} may together form a ring which has three to six ring members and, in addition to carbon atoms, may also contain heteroatoms from the group consisting of O, N and S and may be unsubstituted or substituted by further groups Rb;
   R² together with the group attached to the adjacent carbon atom may also form a five- to ten-membered saturated or partially or fully unsaturated mono- or bicyclic ring which, in addition to carbon atoms, may contain 1, 2 or 3 heteroatoms selected from the group consisting of O, N and S and may be substituted by further groups Rb;
R³ is hydrogen, halogen, cyano, nitro, C₁-C₄-alkyl, C₁-C₄-haloalkyl, C₁-C₄-alkoxy, C₁-C₄-haloalkoxy, C₂-C₄-alkenyl, C₂-C₄-alkynyl, C₂-C₄-alkenyloxy, C₂-C₄-alkynyloxy, S(O)ₙR^{bb};
R⁴ is hydrogen, halogen or C₁-C₄-haloalkyl;
R⁵ is hydrogen, C₁-C₄-alkyl, C₁-C₄-haloalkyl, C₁-C₄-alkoxy, C₁-C₄-alkylthio, C₁-C₄-haloalkoxy, C₁-C₄-haloalkylthio;
R⁶, R⁷ independently of one another are hydrogen, halogen or C₁-C₄-alkyl;
Y is O or S;
X is O, S or N—R^{x};
   R^{x} is hydrogen, C₁-C₆-alkyl, C₁-C₄-haloalkyl, C₂-C₆-alkenyl, C₃-C₆-alkynyl, Z—C₃-C₁₀-cycloalkyl, C₁-C₆-alkoxy-C₁-C₆-alkyl, C₁-C₆-cyanoalkyl, Z-phenyl, Z—C(═O)—Ra² or triC₁-C₄-alkylsilyl;
      R^{a2} is C₁-C₆-alkyl, C₁-C₄-haloalkyl, Z—C₁-C₆-alkoxy, Z—C₁-C₄-haloalkoxy or NRⁱRⁱⁱ;
         where in the groups R^{A} and their subsubstituents, the carbon chains and/or the cyclic groups may be partially or fully substituted by groups R^{b},
         or an N-oxide or an agriculturally suitable salt thereof.

The coumarone-derivatives of the present invention are often best applied in conjunction with one or more other HPPD- and/or HST targeting herbicides to obtain control of a wider variety of undesirable vegetation. When used in conjunction with other HPPD- and/or HST targeting herbicides, the presently claimed compounds can be formulated with the other herbicide or herbicides, tank mixed with the other herbicide or herbicides, or applied sequentially with the other herbicide or herbicides.

Some of the herbicides that are useful in conjunction with the coumarone-derivatives of the present invention include benzobicyclon, mesotrione, sulcotrione, tefuryltrione, tembotrione, 4-hydroxy-3-[[2-(2-methoxyethoxy)methyl]-6-(trifluoromethyl)-3-pyridinyl]carbonyl]-bicyclo[3.2.1]-oct-3-en-2-one (bicyclopyrone), ketospiradox or the free acid thereof, benzofenap, pyrasulfotole, pyrazolynate, pyrazoxyfen, topramezone, [2-chloro-3-(2-methoxyethoxy)-4-(methylsulfonyl)phenyly](I-ethyl-5-hydroxy-1H-pyrazol-4-yl)-methanone, (2,3-dihydro-3,3,4-trimethyl-1,1-dioxidobenzo[b]thien-5-yl)(5-hydroxy-1-methyl-1H-pyrazol-4-yl)-methanone, isoxachlortole, isoxaflutole, α-(cyclopropylcarbonyl)-2-(methylsulfonyl)-β-oxo-4-chloro-benzenepropanenitrile, and α-(cyclopropylcarbonyl)-2-(methylsulfonyl)-β-oxo-4-(trifluoromethyl)-benzenepropanenitrile.

In a preferred embodiment the additional herbicide is topramezone.

In a particularly preferred embodiment the additional herbicide is
(1-Ethyl-5-prop-2-ynyloxy-1H-pyrazol-4-yl)-[4-methansulfonyl-2-methyl-3-(3-methyl-4,5-dihydro-isoxazol-5-yl)-phenyl]-methanon

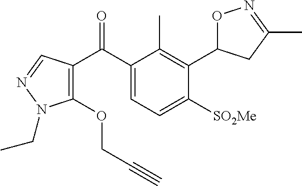 [SMILES: C#CCOc1c(C(=O)c2ccc(S(C)(=O)=O)c(C3CC(C)=NO3)c2C)cnn1CC]

or
(1-Ethyl-5-hydroxy-1H-pyrazol-4-yl)-[4-methansulfonyl-2-methyl-3-(3-methyl-4,5-dihydro-isoxazol-5-yl)-phenyl]-methanon

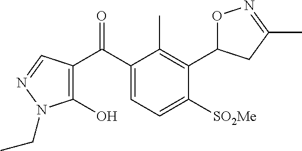 [SMILES: CCn1ncc(C(=O)c2ccc(S(C)(=O)=O)c(C3CC(C)=NO3)c2C)c1O]

The above described compounds are described in great detail in EP 09177628.6 which is entirely incorporated herein by reference.

The herbicidal compounds of the present invention may further be used in conjunction with additional herbicides to which the crop plant is naturally tolerant, or to which it is resistant via expression of one or more additional transgenes as mentioned supra. Some of the herbicides that can be employed in conjunction with the compounds of the present invention include sulfonamides such as metosulam, flumetsulam, cloransulam-methyl, diclosulam, penoxsulam and florasulam, sulfonylureas such as chlorimuron, tribenuron, sulfometuron, nicosulfuron, chlorsulfuron, amidosulfuron, triasulfuron, prosulfuron, tritosulfuron, thifensulfuron, sulfosulfuron and metsulfuron, imidazolinones such as imazaquin, imazapic, ima-zethapyr, imzapyr, imazamethabenz and imazamox, phenoxyalkanoic acids such as 2,4-D, MCPA, dichlorpropand mecoprop, pyridinyloxyacetic acids such as triclopyr and fluoroxypyr, carboxylic acids such as clopyralid, picloram, aminopyralid and dicamba, dinitroanilines such as trifluralin, benefin, benfluralin and pendimethalin, chloroacetanilides such as alachlor, acetochlor and metolachlor, semicarbazones (auxin transport inhibitors) such as chlorflurenol and diflufenzopyr, aryloxyphenoxypropionates such as fluazifop, haloxyfop, diclofop, clodinafop and fenoxapropand other common herbicides including glyphosate, glufosinate, acifluorfen, bentazon, clomazone, fumiclorac, fluometuron, fomesafen, lactofen, linuron, isoproturon, simazine, norflurazon, paraquat, diuron, diflufenican, picolinafen, cinidon, sethoxydim, tralkoxydim, quinmerac, isoxaben, bromoxynil, metribuzin and mesotrione.

The coumarone-derivative herbicides of the present invention can, further, be used in conjunction with glyphosate and glufosinate on glyphosate-tolerant or glufosinate-tolerant crops.

Unless already included in the disclosure above, the coumarone-derivative herbicides of the present invention can, further, be used in conjunction with compounds:

(a) from the group of Lipid Biosynthesis Inhibitors:

Alloxydim, Alloxydim-natrium, Butroxydim, Clethodim, Clodinafop, Clodinafop-propargyl, Cycloxydim, Cyhalofop, Cyhalofop-butyl, Diclofop, Diclofop-methyl, Fenoxaprop, Fenoxapropethyl, Fenoxaprop-P, Fenoxaprop-P-ethyl, Fluazifop, Fluazifop-butyl, Fluazifop-P, FluazifopP-butyl, Haloxyfop, Haloxyfop-methyl, Haloxyfop-P, Haloxyfop-P-methyl, Metamifop, Pinoxaden, Profoxydim, Propaquizafop, Quizalofop, Quizalofop-ethyl, Quizalofop-tefuryl, Quizalofop-P, Quizalofop-P-ethyl, Quizalofop-P-tefuryl, Sethoxydim, Tepraloxydim, Tralkoxydim, Benfuresat, Butylat, Cycloat, Dalapon, Dimepiperat, EPTC, Esprocarb, Ethofumesat, Flupropanat, Molinat, Orbencarb, Pebulat, Prosulfocarb, TCA, Thiobencarb, Tiocarbazil, Triallat and Vernolat;

(b) from the group of ALS-Inhibitors:

Amidosulfuron, Azimsulfuron, Bensulfuron, Bensulfuron-methyl, Bispyribac, Bispyribacnatrium, Chlorimuron, Chlorimuron-ethyl, Chlorsulfuron, Cinosulfuron, Cloransulam, Cloransulam-methyl, Cyclosulfamuron, Diclosulam, Ethametsulfuron, Ethametsulfuron-methyl, Ethoxysulfuron, Flazasulfuron, Florasulam, Flucarbazon, Flucarbazon-natrium, Flucetosulfuron, Flumetsulam, Flupyrsulfuron, Flupyrsulfuron-methyl-natrium, Foramsulfuron, Halosulfuron, Halosulfuron-methyl, Imazamethabenz, Imazamethabenz-methyl, Imazamox, Imazapic, Imazapyr, Imazaquin, Imazethapyr, Imazosulfuron, Iodosulfuron, Iodosulfuron-methyl-natrium, Mesosulfuron, Metosulam, Metsulfuron, Metsulfuron-methyl, Nicosulfuron, Orthosulfamuron, Oxasulfuron, Penoxsulam, Primisulfuron, Primisulfuron-methyl, Propoxycarbazon, Propoxycarbazon-natrium, Prosulfuron, Pyrazosulfuron, Pyrazosulfuron-ethyl, Pyribenzoxim, Pyrimisulfan, Pyriftalid, Pyriminobac, Pyriminobac-methyl, Pyrithiobac, Pyrithiobac-natrium, Pyroxsulam, Rimsulfuron, Sulfometuron, Sulfometuron-methyl, Sulfosulfuron, Thiencarbazon, Thiencarbazon-methyl, Thifensulfuron, Thifensulfuron-methyl, Triasulfuron, Tribenuron, Tribenuron-methyl, Trifloxysulfuron, Triflusulfuron, Triflusulfuron-methyl and Tritosulfuron;

(c) from the group of Photosynthese-Inhibitors:

Ametryn, Amicarbazon, Atrazin, Bentazon, Bentazon-natrium, Bromacil, Bromofenoxim, Bromoxynil and its salts and esters, Chlorobromuron, Chloridazon, Chlorotoluron, Chloroxuron, Cyanazin, Desmedipham, Desmetryn, Dimefuron, Dimethametryn, Diquat, Diquatdibromid, Diuron, Fluometuron, Hexazinon, loxynil and its salts and esters, Isoproturon, Isouron, Karbutilat, Lenacil, Linuron, Metamitron, Methabenzthiazuron, Metobenzuron, Metoxuron, Metribuzin, Monolinuron, Neburon, Paraquat, Paraquat-dichlorid, Paraquatdimetilsulfat, Pentanochlor, Phenmedipham, Phenmedipham-ethyl, Prometon, Prometryn, Propanil, Propazin, Pyridafol, Pyridat, Siduron, Simazin, Simetryn, Tebuthiuron, Terbacil, Terbumeton, Terbuthylazin, Terbutryn, Thidiazuron and Trietazin;

d) from the group of Protoporphyrinogen-IX-Oxidase-Inhibitors:

Acifluorfen, Acifluorfen-natrium, Azafenidin, Bencarbazon, Benzfendizon, Bifenox, Butafenacil, Carfentrazon, Carfentrazon-ethyl, Chlomethoxyfen, Cinidon-ethyl, Fluazolat, Flufenpyr, Flufenpyr-ethyl, Flumiclorac, Flumiclorac-pentyl, Flumioxazin, Fluoroglycofen, Fluoroglycofen-ethyl, Fluthiacet, Fluthiacet-methyl, Fomesafen, Halosafen, Lactofen, Oxadiargyl, Oxadiazon, Oxyfluorfen, Pentoxazon, Profluazol, Pyraclonil, Pyraflufen, Pyraflufen-ethyl, Saflufenacil, Sulfentrazon, Thidiazimin, 2-Chlor-5-[3,6-dihydro-3-methyl-2,6-dioxo-4-(trifluormethyl)-[(2H)-pyrimidinyl]-4-fluor-N-[(isopropyl)methylsulfamoyl]benzamid (H-1; CAS 372137-35-4), [3-[2-Chlor-4-fluor-5-(1-methyl-6-trifluormethyl-2,4-dioxo-1,2,3,4,-tetrahydropyrimidin-3-yl)phenoxy]-2-pyridyloxy]acetic acidethylester (H-2; CAS 353292-31-6), N-Ethyl-3-(2,6-dichlor-4-trifluormethylphenoxy)-5-methyl-1H-pyrazol-1-carboxamid (H-3; CAS 452098-92-9), N-Tetrahydrofurfuryl-3-(2,6-dichlor-4-trifluormethylphenoxy)-5-methyl-1H-pyrazol-1-carboxamid (H-4; CAS 915396-43-9), N-Ethyl-3-(2-chlor-6-fluor-4-trifluormethylphenoxy)-5-methyl-1H-pyrazol-1-carboxamid (H-5; CAS 452099-05-7) and N-Tetrahydrofurfuryl-3-(2-chlor-6-fluor-4-trifluormethylphenoxy)-5-methyl-1H-pyrazol-1-carboxamid (H-6; CAS 45100-03-7);

e) from the group of Bleacher-Herbicides:

Aclonifen, Amitrol, Beflubutamid, Benzobicyclon, Benzofenap, Clomazon, Diflufenican, Fluridon, Fluorochloridon, Flurtamon, Isoxaflutol, Mesotrion, Norflurazon, Picolinafen, Pyrasulfutol, Pyrazolynat, Pyrazoxyfen, Sulcotrion, Tefuryltrion, Tembotrion, Topramezon, 4-Hydroxy-3-[[2-[(2-methoxyethoxy)methyl]-6-(trifluormethyl)-3-pyridyl]carbonyl]bicyclo[3.2.1]oct-3-en-2-one (H-7; CAS 352010-68-5) and 4-(3-Trifluormethylphenoxy)-2-(4-trifluormethylphenyl)pyrimidin (H-8; CAS180608-33-7);

f) from the group of EPSP-Synthase-Inhibitors:

Glyphosat, Glyphosat-isopropylammonium and Glyphosat-trimesium (Sulfosat);

g) from the group of Glutamin-Synthase-Inhibitors:

Bilanaphos (Bialaphos), Bilanaphos-natrium, Glufosinat and Glufosinat-ammonium;

h) from the group of DHP-Synthase-Inhibitors: Asulam;
i) from the group of Mitose-Inhibitors:

Amiprophos, Amiprophos-methyl, Benfluralin, Butamiphos, Butralin, Carbetamid, Chlorpropham, Chlorthal, Chlorthal-dimethyl, Dinitramin, Dithiopyr, Ethalfluralin, Fluchloralin, Oryzalin, Pendimethalin, Prodiamin, Propham, Propyzamid, Tebutam, Thiazopyr and Trifluralin;

j) from the group of VLCFA-Inhibitors:

Acetochlor, Alachlor, Anilofos, Butachlor, Cafenstrol, Dimethachlor, Dimethanamid, Dimethenamid-P, Diphenamid, Fentrazamid, Flufenacet, Mefenacet, Metazachlor, Metolachlor, Metolachlor-S, Naproanilid, Napropamid, Pethoxamid, Piperophos, Pretilachlor, Propachlor, Propisochlor, Pyroxasulfon (KIH-485) and Thenylchlor;

Compounds of the Formula 2:

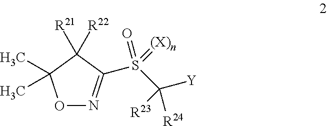

Particularly preferred Compounds of the formula 2 are: 3-[5-(2,2-Difluor-ethoxy)-1-methyl-3-trifluormethyl-1H-pyrazol-4-ylmethansulfonyl]-4-fluor-5,5-dimethyl-4,5-dihydro-isoxazol (2-1); 3-{[5-(2,2-Difluor-ethoxy)-1-methyl-3-trifluormethyl-1H-pyrazol-4-yl]-fluor-methansulfonyl}-5,5-dimethyl-4,5-dihydro-isoxazol (2-2); 4-(4-Fluor-5,5-dimethyl-4,5-dihydro-isoxazol-3-sulfonylmethyl)-2-methyl-5-trifluormethyl-2H-[1,2,3]triazol (2-3); 4-[(5,5-Dimethyl-4,5-dihydro-isoxazol-3-sulfonyl)-fluor-methyl]-2-methyl-5-trifluormethyl-2H-[1,2,3]triazol (2-4); 4-(5,5-Dimethyl-4,5-dihydro-isoxazol-3-sulfonylmethyl)-2-methyl-5-trifluormethyl-2H-[1,2,3]triazol (2-5); 3-{[5-(2,2-Difluor-ethoxy)-1-methyl-3-trifluormethyl-1H-pyrazol-4-yl]-difluor-methansulfonyl}-5,5-dimethyl-4,5-dihydro-isoxazol (2-6); 4-[(5,5-Dimethyl-4,5-dihydro-isoxazol-3-sulfonyl)-difluor-methyl]-2-methyl-5-trifluormethyl-2H-[1,2,3]triazol (2-7); 3-{[5-(2,2-Difluor-ethoxy)-1-methyl-3-trifluormethyl-1H-pyrazol-4-yl]-difluormethansulfonyl}-4-fluor-5,5-dimethyl-4,5-dihydro-isoxazol (2-8); 4-[Difluor-(4-fluor-5,5-dimethyl-4,5-dihydro-isoxazol-3-sulfonyl)-methyl]-2-methyl-5-trifluormethyl-2H-[1,2,3]triazol (2-9);

k) from the Group of Cellulose-Biosynthese-Inhibitors:

Chlorthiamid, Dichlobenil, Flupoxam and Isoxaben;

l) from the group of Uncoupling-Herbicides:

Dinoseb, Dinoterb and DNOC and its salts;

m) from the group of Auxin-Herbicides:

2,4-D and its salts and esters, 2,4-DB and its salts and esters, Aminopyralid and its salts wie Aminopyralid-tris(2-hydroxypropyl)ammonium and its esters, Benazolin, Benazolin-ethyl, Chloramben and its salts and esters, Clomeprop, Clopyralid and its salts and esters, Dicamba and its salts and esters, Dichlorpropand its salts and esters, Dichlorprop-P and its salts and esters, Fluoroxypyr, Fluoroxypyr-butomethyl, Fluoroxypyr-meptyl, MCPA and its salts and esters, MCPA-thioethyl, MCPB and its salts and esters, Mecopropand its salts and esters, Mecoprop-P and its salts and esters, Picloram and its salts and esters, Quinclorac, Quinmerac, TBA (2,3,6) and its salts and esters, Triclopyr and its salts and esters, and 5,6-Dichlor-2-cyclopropyl-4-pyrimidincarbonic acid (H-9; CAS 858956-08-8) and its salts and esters;

n) from the group of Auxin-Transport-Inhibitors: Diflufenzopyr, Diflufenzopyr-natrium, Naptalam and Naptalam-natrium;
o) from the group of other Herbicides: Bromobutid, Chlorflurenol, Chlorflurenol-methyl, Cinmethylin, Cumyluron, Dalapon, Dazomet, Difenzoquat, Difenzoquat-metilsulfate, Dimethipin, DSMA, Dymron, Endothal and its salts, Etobenzanid, Flamprop, Flamprop-isopropyl, Flamprop-methyl Flamprop-M-isopropyl, Flamprop-M-methyl, Flurenol, Flurenol-butyl, Flurprimidol, Fosamin, Fosamine-ammonium, Indanofan, Maleinic acid-hydrazid, Mefluidid, Metam, Methylazid, Methylbromid, Methyl-dymron, Methyljodid. MSMA, oleic acid, Oxaziclomefon, Pelargonic acid, Pyributicarb, Quinoclamin, Triaziflam, Tridiphan and 6-Chlor-3-(2-cyclopropyl-6-methylphenoxy)-4-pyridazinol (H-10; CAS 499223-49-3) and its salts and esters.

Examples for preferred Safeners C are Benoxacor, Cloquintocet, Cyometrinil, Cyprosulfamid, Dichlormid, Dicyclonon, Dietholate, Fenchlorazol, Fenclorim, Flurazol, Fluxofenim, Furilazol, Isoxadifen, Mefenpyr, Mephenat, Naphthalic acid anhydrid, Oxabetrinil, 4-(Dichloracetyl)-1-oxa-4-azaspiro[4.5]decan (H-11; MON4660, CAS 71526-07-3) and 2,2,5-Trimethyl-3-(dichloracetyl)-1,3-oxazolidin (H-12; R-29148, CAS 52836-31-4).

The compounds of groups a) to o) and the Safeners C are known Herbicides and Safeners, see e.g. The Compendium of Pesticide Common Names (http://www.alanwood.net/pesticides/); B. Hock, C. Fedtke, R. R. Schmidt, Herbicides, Georg Thieme Verlag, Stuttgart 1995. Other herbicidal effectors are known from WO 96/26202, WO 97/41116, WO 97/41117, WO 97/41118, WO 01/83459 and WO 2008/074991 as well as from W. Kramer et al. (ed.) “Modern Crop Protection Compounds”, Vol. 1, Wiley VCH, 2007 and the literature cited therein.

It is generally preferred to use the compounds of the invention in combination with herbicides that are selective for the crop being treated and which complement the spectrum of weeds controlled by these compounds at the application rate employed. It is further generally preferred to apply the compounds of the invention and other complementary herbicides at the same time, either as a combination formulation or as a tank mix.

The term “mut-HPPD nucleic acid” refers to an HPPD nucleic acid having a sequence that is mutated from a wild-type HPPD nucleic acid and that confers increased “coumarone-derivative herbicide” tolerance to a plant in which it is expressed. Furthermore, the term “mutated hydroxyphenyl pyruvate dioxygenase (mut-HPPD)” refers to the replacement of an amino acid of the wild-type primary sequences SEQ ID NO: 2, 4, 6, 11, 12, 13, 14, 15, 16, 17, 18, 19, a variant, a derivative, a homologue, an orthologue, or paralogue thereof, with another amino acid. The expression “mutated amino acid” will be used below to designate the amino acid which is replaced by another amino acid, thereby designating the site of the mutation in the primary sequence of the protein.

The term “mut-HST nucleic acid” refers to an HST nucleic acid having a sequence that is mutated from a wild-type HST nucleic acid and that confers increased “coumarone-derivative herbicide” tolerance to a plant in which it is expressed. Furthermore, the term “mutated homogentisate solanesyl transferase (mut-HST)” refers to the replacement of an amino acid of the wild-type primary sequences SEQ ID NO: 8 or 10 with another amino acid. The expression “mutated amino acid” will be used below to designate the amino acid which is replaced by another amino acid, thereby designating the site of the mutation in the primary sequence of the protein.

Several HPPDs and their primary sequences have been described in the state of the art, in particular the HPPDs of bacteria such as Pseudomonas (Ruetschi et al., Eur. J. Biochem., 205, 459-466, 1992, WO96/38567), of plants such as Arabidopsis (WO96/38567, Genebank AF047834) or of carrot (WO96/38567, Genebank 87257) of Coccicoides (Genebank COITRP), HPPDs of Arabidopsis, Brassica, cotton, Synechocystis, and tomato (U.S. Pat. No. 7,297,541), of mammals such as the mouse or the pig. Furthermore, artificial HPPD sequences have been described, for example in U.S. Pat. No. 6,768,044; U.S. Pat. No. 6,268,549;

In a preferred embodiment, the nucleotide sequence of (i) comprises the sequence of SEQ ID NO: 1, 3, or 5 or a variant or derivative thereof.

In another preferred embodiment, the nucleotide sequence of (ii) comprises the sequence of SEQ ID NO: 7 or 9, or a variant or derivative thereof.

Furthermore, it will be understood by the person skilled in the art that the nucleotide sequences of (i) or (ii) encompasse homologues, paralogues and orthologues of SEQ ID NO: 1, 3, or 5, and respectively SEQ ID NO: 7 or 9, as defined hereinafter.

The term “variant” with respect to a sequence (e.g., a polypeptide or nucleic acid sequence such as—for example—a transcription regulating nucleotide sequence of the invention) is intended to mean substantially similar sequences. For nucleotide sequences comprising an open reading frame, variants include those sequences that, because of the degeneracy of the genetic code, encode the identical amino acid sequence of the native protein. Naturally occurring allelic variants such as these can be identified with the use of well-known molecular biology techniques, as, for example, with polymerase chain reaction (PCR) and hybridization techniques. Variant nucleotide sequences also include synthetically derived nucleotide sequences, such as those generated, for example, by using site-directed mutagenesis and for open reading frames, encode the native protein, as well as those that encode a polypeptide having amino acid substitutions relative to the native protein. Generally, nucleotide sequence variants of the invention will have at least 30, 40, 50, 60, to 70%, e.g., preferably 71%, 72%, 73%, 74%, 75%, 76%, 77%, 78%, to 79%, generally at least 80%, e.g., 81%-84%, at least 85%, e.g., 86%, 87%, 88%, 89%, 90%, 91%, 92%, 93%, 94%, 95%, 96%, 97%, to 98% and 99% nucleotide “sequence identity” to the nucleotide sequence of SEQ ID NO:1, 3, 5, 7, or 9. By “variant” polypeptide is intended a polypeptide derived from the protein of SEQ ID NO:2, 4, 6, 8, or 10 by deletion (so-called truncation) or addition of one or more amino acids to the N-terminal and/or C-terminal end of the native protein; deletion or addition of one or more amino acids at one or more sites in the native protein; or substitution of one or more amino acids at one or more sites in the native protein. Such variants may result from, for example, genetic polymorphism or from human manipulation. Methods for such manipulations are generally known in the art.

In a particularly preferred embodiment, site-directed mutagenesis for generating a variant of HPPD of SEQ ID NO: 2 is carried out by using one or more of the primers selected from the group consisting of SEQ ID NOs: 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67.

It is recognized that the polynucleotide molecules and polypeptides of the invention encompass polynucleotide molecules and polypeptides comprising a nucleotide or an amino acid sequence that is sufficiently identical to nucleotide sequences set forth in SEQ ID Nos: 1, 3, 5, 7, or 9, or to the amino acid sequences set forth in SEQ ID Nos: 2, 4, 6, 8, 10, 11, 12, 13, 14, 15, 16, 17, 18, or 19. The term “sufficiently identical” is used herein to refer to a first amino acid or nucleotide sequence that contains a sufficient or minimum number of identical or equivalent (e.g., with a similar side chain) amino acid residues or nucleotides to a second amino acid or nucleotide sequence such that the first and second amino acid or nucleotide sequences have a common structural domain and/or common functional activity.

“Sequence identity” refers to the extent to which two optimally aligned DNA or amino acid sequences are invariant throughout a window of alignment of components, e.g., nucleotides or amino acids. An “identity fraction” for aligned segments of a test sequence and a reference sequence is the number of identical components that are shared by the two aligned sequences divided by the total number of components in reference sequence segment, i.e., the entire reference sequence or a smaller defined part of the reference sequence. “Percent identity” is the identity fraction times 100. Optimal alignment of sequences for aligning a comparison window are well known to those skilled in the art and may be conducted by tools such as the local homology algorithm of Smith and Waterman, the homology alignment algorithm of Needleman and Wunsch, the search for similarity method of Pearson and Lipman, and preferably by computerized implementations of these algorithms such as GAP, BESTFIT, FASTA, and TFASTA available as part of the GCG. Wisconsin Package. (Accelrys Inc. Burlington, Mass.)

The terms “polynucleotide(s)”, “nucleic acid sequence(s)”, “nucleotide sequence(s)”, “nucleic acid(s)”, “nucleic acid molecule” are used interchangeably herein and refer to nucleotides, either ribonucleotides or deoxyribonucleotides or a combination of both, in a polymeric unbranched form of any length.

“Derivatives” of a protein encompass peptides, oligopeptides, polypeptides, proteins and enzymes having amino acid substitutions, deletions and/or insertions relative to the unmodified protein in question and having similar biological and functional activity as the unmodified protein from which they are derived.

“Homologues” of a protein encompass peptides, oligopeptides, polypeptides, proteins and enzymes having amino acid substitutions, deletions and/or insertions relative to the unmodified protein in question and having similar biological and functional activity as the unmodified protein from which they are derived.

A deletion refers to removal of one or more amino acids from a protein.

An insertion refers to one or more amino acid residues being introduced into a predetermined site in a protein. Insertions may comprise N-terminal and/or C-terminal fusions as well as intra-sequence insertions of single or multiple amino acids. Generally, insertions within the amino acid sequence will be smaller than N- or C-terminal fusions, of the order of about 1 to 10 residues. Examples of N- or C-terminal fusion proteins or peptides include the binding domain or activation domain of a transcriptional activator as used in the yeast two-hybrid system, phage coat proteins, (histidine)-6-tag, glutathione S-transferase-tag, protein A, maltose-binding protein, dihydrofolate reductase, Tag•100 epitope, c-myc epitope, FLAG®-epitope, lacZ, CMP (calmodulin-binding peptide), HA epitope, protein C epitope and VSV epitope.

A substitution refers to replacement of amino acids of the protein with other amino acids having similar properties (such as similar hydrophobicity, hydrophilicity, antigenicity, propensity to form or break α-helical structures or β-sheet structures). Amino acid substitutions are typically of single residues, but may be clustered depending upon functional constraints placed upon the polypeptide and may range from 1 to 10 amino acids; insertions will usually be of the order of about 1 to 10 amino acid residues. The amino acid substitutions are preferably conservative amino acid substitutions. Conservative substitution tables are well known in the art (see for example Creighton (1984) Proteins. W.H. Freeman and Company (Eds).

TABLE 3
Examples of conserved amino acid substitutions

| | Conservative |
|---|---|
| Residue | Substitutions |
| Ala | Ser |
| Arg | Lys |
| Asn | Gln; His |
| Asp | Glu |
| Gln | Asn |
| Cys | Ser |
| Glu | Asp |
| Gly | Pro |
| His | Asn; Gln |
| Ile | Leu, Val |
| Leu | Ile; Val |
| Lys | Arg; Gln |
| Met | Leu; Ile |
| Phe | Met; Leu; Tyr |
| Ser | Thr; Gly |
| Thr | Ser; Val |
| Trp | Tyr |
| Tyr | Trp; Phe |
| Val | Ile; Leu |

Amino acid substitutions, deletions and/or insertions may readily be made using peptide synthetic techniques well known in the art, such as solid phase peptide synthesis and the like, or by recombinant DNA manipulation. Methods for the manipulation of DNA sequences to produce substitution, insertion or deletion variants of a protein are well known in the art. For example, techniques for making substitution mutations at predetermined sites in DNA are well known to those skilled in the art and include M13 mutagenesis, T7-Gen in vitro mutagenesis (USB, Cleveland, Ohio), QuickChange Site Directed mutagenesis (Stratagene, San Diego, Calif.), PCR-mediated site-directed mutagenesis or other site-directed mutagenesis protocols.

“Derivatives” further include peptides, oligopeptides, polypeptides which may, compared to the amino acid sequence of the naturally-occurring form of the protein, such as the protein of interest, comprise substitutions of amino acids with non-naturally occurring amino acid residues, or additions of non-naturally occurring amino acid residues. “Derivatives” of a protein also encompass peptides, oligopeptides, polypeptides which comprise naturally occurring altered (glycosylated, acylated, prenylated, phosphorylated, myristoylated, sulphated etc.) or non-naturally altered amino acid residues compared to the amino acid sequence of a naturally-occurring form of the polypeptide. A derivative may also comprise one or more non-amino acid substituents or additions compared to the amino acid sequence from which it is derived, for example a reporter molecule or other ligand, covalently or non-covalently bound to the amino acid sequence, such as a reporter molecule which is bound to facilitate its detection, and non-naturally occurring amino acid residues relative to the amino acid sequence of a naturally-occurring protein. Furthermore, “derivatives” also include fusions of the naturally-occurring form of the protein with tagging peptides such as FLAG, HIS6 or thioredoxin (for a review of tagging peptides, see Terpe, Appl. Microbiol. Biotechnol. 60, 523-533, 2003).

“Orthologues” and “paralogues” encompass evolutionary concepts used to describe the ancestral relationships of genes. Paralogues are genes within the same species that have originated through duplication of an ancestral gene; orthologues are genes from different organisms that have originated through speciation, and are also derived from a common ancestral gene. A non-limiting list of examples of such orthologues is shown in Table 1.

It is well-known in the art that paralogues and orthologues may share distinct domains harboring suitable amino acid residues at given sites, such as binding pockets for particular substrates or binding motifs for interaction with other proteins.

The term “domain” refers to a set of amino acids conserved at specific positions along an alignment of sequences of evolutionarily related proteins. While amino acids at other positions can vary between homologues, amino acids that are highly conserved at specific positions indicate amino acids that are likely essential in the structure, stability or function of a protein. Identified by their high degree of conservation in aligned sequences of a family of protein homologues, they can be used as identifiers to determine if any polypeptide in question belongs to a previously identified polypeptide family.

The term “motif” or “consensus sequence” refers to a short conserved region in the sequence of evolutionarily related proteins. Motifs are frequently highly conserved parts of domains, but may also include only part of the domain, or be located outside of conserved domain (if all of the amino acids of the motif fall outside of a defined domain).

Specialist databases exist for the identification of domains, for example, SMART (Schultz et al. (1998) Proc. Natl. Acad. Sci. USA 95, 5857-5864; Letunic et al. (2002) Nucleic Acids Res 30, 242-244), InterPro (Mulder et al., (2003) Nucl. Acids. Res. 31, 315-318), Prosite (Bucher and Bairoch (1994), A generalized profile syntax for biomolecular sequences motifs and its function in automatic sequence interpretation. (In) ISMB-94; Proceedings 2nd International Conference on Intelligent Systems for Molecular Biology. Altman R., Brutlag D., Karp P., Lathrop R., Searls D., Eds., pp 53-61, AAAI Press, Menlo Park; Hulo et al., Nucl. Acids. Res. 32:D134-D137, (2004)), or Pfam (Bateman et al., Nucleic Acids Research 30(1): 276-280 (2002)). A set of tools for in silico analysis of protein sequences is available on the ExPASy proteomics server (Swiss Institute of Bioinformatics (Gasteiger et al., ExPASy: the proteomics server for in-depth protein knowledge and analysis, Nucleic Acids Res. 31:3784-3788 (2003)). Domains or motifs may also be identified using routine techniques, such as by sequence alignment.

Methods for the alignment of sequences for comparison are well known in the art, such methods include GAP, BESTFIT, BLAST, FASTA and TFASTA. GAP uses the algorithm of Needleman and Wunsch ((1970) J Mol Biol 48: 443-453) to find the global (i.e. spanning the complete sequences) alignment of two sequences that maximizes the number of matches and minimizes the number of gaps. The BLAST algorithm (Altschul et al. (1990) J Mol Biol 215: 403-10) calculates percent sequence identity and performs a statistical analysis of the similarity between the two sequences. The software for performing BLAST analysis is publicly available through the National Centre for Biotechnology Information (NCBI). Homologues may readily be identified using, for example, the ClustalW multiple sequence alignment algorithm (version 1.83), with the default pairwise alignment parameters, and a scoring method in percentage. Global percentages of similarity and identity may also be determined using one of the methods available in the MatGAT software package (Campanella et al., BMC Bioinformatics. 2003 Jul. 10; 4:29. MatGAT: an application that generates similarity/identity matrices using protein or DNA sequences). Minor manual editing may be performed to optimise alignment between conserved motifs, as would be apparent to a person skilled in the art. Furthermore, instead of using full-length sequences for the identification of homologues, specific domains may also be used. The sequence identity values may be determined over the entire nucleic acid or amino acid sequence or over selected domains or conserved motif(s), using the programs mentioned above using the default parameters. For local alignments, the Smith-Waterman algorithm is particularly useful (Smith T F, Waterman M S (1981) J. Mol. Biol. 147(1); 195-7).

The inventors of the present invention have surprisingly found that by substituting one or more of the key amino acid residues the herbicide tolerance or resistance could be remarkably increased as compared to the activity of the wild type HPPD enzymes with SEQ ID NO: 2, 4 or 6. Preferred substitutions of mut-HPPD are those that increase the herbicide tolerance of the plant, but leave the biological activitiy of the dioxygenase activity substantially unaffected.

Accordingly, in another object of the present invention the key amino acid residues of a HPPD enzyme, a variant, derivative, othologue, paralogue or homologue thereof, is substituted by any other amino acid.

In a preferred embodiment, the key amino acid residues of a HPPD enzyme, a variant, derivative, othologue, paralogue or homologue thereof, is substituted by a conserved amino acid as depicted in Table 3 above.

It will be understood by the person skilled in the art that amino acids located in a close proximity to the positions of amino acids mentioned below may also be substituted. Thus, in another embodiment the variant of SEQ ID NO:2, 4, 6, 11, 12, 13, 14, 15, 16, 17, 18, 19, a variant, derivative, orthologue, paralogue or homologue thereof comprises a mut-HPPD, wherein an amino acid ±3, ±2 or ±1 amino acid positions from a key amino acid is substituted by any other amino acid.

Based on techniques well-known in the art, a highly characteristic sequence pattern can be developed, by means of which further of mut-HPPD candidates with the desired activity may be searched.

Searching for further mut-HPPD candidates by applying a suitable sequence pattern would also be encompassed by the present invention. It will be understood by a skilled reader that the present sequence pattern is not limited by the exact distances between two adjacent amino acid residues of said pattern. Each of the distances between two neighbours in the above patterns may, for example, vary independently of each other by up to ±10, ±5, ±3, ±2 or ±1 amino acid positions without substantially affecting the desired activity.

In line with said above functional and spatial analysis of individual amino acid residues based on the crystallographic data as obtained according to the present invention, unique partial amino acid sequences characteristic of potentially useful mut-HPPD candidates of the invention may be identified.

In a particularly preferred embodiment, the variant or derivative of the mut-HPPD of SEQ ID NO: 2 is selected from the following Table 4a and combined amino acid substitutions of mut-HPPD of SEQ ID NO: 2 are selected from Table 4b.

TABLE 4a
(Sequence ID No: 2): single amino acid substitutions

| Key amino | | Preferred |
|---|---|---|
| acid position | Substituents | substituents |
| Gln293 | Ala, Leu, Ile, Val, His, Asn | Val, His, Asn |
| Met335 | Ala, Trp, Phe, Leu, Ile, Val, Asn, Gln | Ala, Trp, Phe |
| Pro336 | Ala | Ala |
| Ser337 | Ala, Pro | Ala, Pro |
| Phe392 | Ala, Leu | Ala |
| Glu363 | Gln | Gln |
| Gly422 | His, Met, Phe, Cys | |
| Leu427 | Phe, Trp | Phe |
| Thr382 | Pro | Pro |
| Leu385 | Ala, Val | Val |
| Ile393 | Ala, Leu | Leu |

TABLE 4b
(Sequence ID No: 2): combined amino acid substitutions

| | Key amino | | Preferred |
|---|---|---|---|
| Combination No | acid position | Substituents | substituents |
| 1 | Pro336 | Ala | Ala |
| | Glu363 | Gln | Gln |
| 2 | Thr382 | Pro | Pro |
| | Leu385 | Ala, Val | Val |
| | Ile393 | Ala, Leu | Leu |

It is to be understood that any amino acid besides the ones mentioned in the above table could be used as a substitutent. Assays to test for the functionality of such mutants are readily available in the art, and respectively, described in the Example section of the present invention.

In a preferred embodiment, the amino acid sequence differs from an amino acid sequence of an HPPD of SEQ ID NO: 2 at one or more of the following positions: 293, 335, 336, 337, 392, 363, 422, 427, 382, 385, 393.

Examples of differences at these amino acid positions include, but are not limited to, one or more of the following: the amino acid at position 293 is other than glutamine; the amino acid at position 335 is other than methionine; the amino acid at position 336 is other than proline; the amino acid at position 337 is other than serine; the amino acid position 392 is other than phenylalanine; the amino acid position 363 is other than glutamic acid; the amino acid at position 422 is other than glycine; the amino acid at position 427 is other than leucine; the amino acid position 382 is other than threonine; the amino acid at position 385 is other than leucine; the amino acid position 393 is other than an isoleucine.

In some embodiments, the HPPD enzyme of SEQ ID NO: 2 comprises one or more of the following: the amino acid at position 293 is Alanine, Leucine, Isoleucine, Valine, Histidine, or Asparagine; the amino acid at position 335 is Alanine, Tryptophane, Phenylalanine, Leucine, Isoleucine, Valine, Asparagine, or Glutamine; the amino acid at position 336 is alanine; the amino acid at position 337 is alanine or proline; the amino acid position 392 is alanine or leucine; the amino acid position 363 is glutamine; the amino acid at position 422 is Histidine, Methionine, Phenylalanine, or Cysteine; the amino acid at position 427 is Phenylalanine, or Tryptophan; the amino acid position 382 is proline; the amino acid at position 385 is valine or alanine; the amino acid position 393 is alanine or leucine.

In particular preferred embodiments, the HPPD enzyme of SEQ ID NO: 2 comprises one or more of the following: the amino acid at position 336 is alanine; the amino acid position 363 is glutamine; the amino acid position 393 is leucine; the amino acid at position 385 is valine.

In a further preferred embodiment, the amino acid sequence differs from an amino acid sequence of an HPPD of SEQ ID NO: 6 at position 418. Preferably, the amino acid at position 418 is other alanine. More preferably, the amino acid at position 418 is threonine.

It will be within the knowledge of the skilled artisan to identify conserved regions and motifs shared between the homologues, orthologues and paralogues of SEQ ID NO: 1, 3, or 5, and respectively SEQ ID NO: 7 or 9, such as those depicted in Table 1. Having identified such conserved regions that may represent suitable binding motifs, amino acids corresponding to the amino acids listed in Table 4a and 4b, can be chosen to be substituted by any other amino acid, preferably by conserved amino acids as shown in table 3, and more preferably by the amino acids of tables 4a and 4b.

In addition, the present invention refers to a method for identifying a coumarone-derivative herbicide by using a mut-HPPD encoded by a nucleic acid which comprises the nucleotide sequence of SEQ ID NO: 1, 3, or 5, or a variant or derivative thereof, and/or by using a mut-HST encoded by a nucleic acid which comprises the nucleotide sequence of SEQ ID NO: 7 or 9, or a variant or derivative thereof.

Said method comprises the steps of:
a) generating a transgenic cell or plant comprising a nucleic acid encoding a mut-HPPD, wherein the mut-HPPD is expressed;
b) applying a coumarone-derivative herbicide to the transgenic cell or plant of a) and to a control cell or plant of the same variety;
c) determining the growth or the viability of the transgenic cell or plant and the control cell or plant after application of said coumarone-derivative herbicide, and
d) selecting “coumarone-derivative herbicides” which confer reduced growth to the control cell or plant as compared to the growth of the transgenic cell or plant.

By “control cell” or “similar, wild-type, plant, plant tissue, plant cell or host cell” is intended a plant, plant tissue, plant cell, or host cell, respectively, that lacks the herbicide-resistance characteristics and/or particular polynucleotide of the invention that are disclosed herein. The use of the term “wild-type” is not, therefore, intended to imply that a plant, plant tissue, plant cell, or other host cell lacks recombinant DNA in its genome, and/or does not possess herbicide-resistant characteristics that are different from those disclosed herein.

Another object refers to a method of identifying a nucleotide sequence encoding a mut-HPPD which is resistant or tolerant to a coumarone-derivative herbicide, the method comprising:
a) generating a library of mut-HPPD-encoding nucleic acids,
b) screening a population of the resulting mut-HPPD-encoding nucleic acids by expressing each of said nucleic acids in a cell or plant and treating said cell or plant with a coumarone-derivative herbicide,
c) comparing the coumarone-derivative herbicide-tolerance levels provided by said population of mut-HPPD encoding nucleic acids with the coumarone-derivative herbicide-tolerance level provided by a control HPPD-encoding nucleic acid,
d) selecting at least one mut-HPPD-encoding nucleic acid that provides a significantly increased level of tolerance to a coumarone-derivative herbicide as compared to that provided by the control HPPD-encoding nucleic acid.

In a preferred embodiment, the mut-HPPD-encoding nucleic acid selected in step d) provides at least 2-fold as much resistance or tolerance of a cell or plant to a coumarone-derivative herbicide as compared to that provided by the control HPPD-encoding nucleic acid.

In a further preferred embodiment, the mut-HPPD-encoding nucleic acid selected in step d) provides at least 2-fold, at least 5-fold, at least 10-fold, at least 20-fold, at least 50-fold, at least 100-fold, at least 500-fold, as much resistance or tolerance of a cell or plant to a coumarone-derivative herbicide as compared to that provided by the control HPPD-encoding nucleic acid.

The resistance or tolerance can be determined by generating a transgenic plant or host cell, preferably a plant cell, comprising a nucleic acid sequence of the library of step a) and comparing said transgenic plant with a control plant or host cell, preferably a plant cell.

Another object refers to a method of identifying a plant or algae containing a nucleic acid comprising a nucleotide sequence encoding a mut-HPPD or mut-HST which is resistant or tolerant to a coumarone-derivative herbicide, the method comprising:
a) identifying an effective amount of a coumarone-derivative herbicide in a culture of plant cells or green algae that leads to death of said cells.
b) treating said plant cells or green algae with a mutagenizing agent,
c) contacting said mutagenized cells population with an effective amount of coumarone-derivative herbicide, identified in a),
d) selecting at least one cell surviving these test conditions,
e) PCR-amplification and sequencing of HPPD and/or HST genes from cells selected in d) and comparing such sequences to wild-type HPPD or HST gene sequences, respectively.

In a preferred embodiment, said mutagenizing agent is ethylmethanesulfonate (EMS).

Many methods well known to the skilled artisan are available for obtaining suitable candidate nucleic acids for identifying a nucleotide sequence encoding a mut-HPPD from a variety of different potential source organisms including microbes, plants, fungi, algae, mixed cultures etc. as well as environmental sources of DNA such as soil. These methods include inter alia the preparation of cDNA or genomic DNA libraries, the use of suitably degenerate oligonucleotide primers, the use of probes based upon known sequences or complementation assays (for example, for growth upon tyrosine) as well as the use of mutagenesis and shuffling in order to provide recombined or shuffled mut-HPPD-encoding sequences.

Nucleic acids comprising candidate and control HPPD encoding sequences can be expressed in yeast, in a bacterial host strain, in an alga or in a higher plant such as tobacco or Arabidopsis and the relative levels of inherent tolerance of the HPPD encoding sequences screened according to a visible indicator phenotype of the transformed strain or plant in the presence of different concentrations of the selected coumarone-derivative herbicide. Dose responses and relative shifts in dose responses associated with these indicator phenotypes (formation of brown color, growth inhibition, herbicidal effect etc) are conveniently expressed in terms, for example, of GR50 (concentration for 50% reduction of growth) or MIC (minimum inhibitory concentration) values where increases in values correspond to increases in inherent tolerance of the expressed HPPD. For example, in a relatively rapid assay system based upon transformation of a bacterium such as E. coli, each mut-HPPD encoding sequence may be expressed, for example, as a DNA sequence under expression control of a controllable promoter such as the lacZ promoter and taking suitable account, for example by the use of synthetic DNA, of such issues as codon usage in order to obtain as comparable a level of expression as possible of different HPPD sequences. Such strains expressing nucleic acids comprising alternative candidate HPPD sequences may be plated out on different concentrations of the selected coumarone-derivative herbicide in, optionally, a tyrosine supplemented medium and the relative levels of inherent tolerance of the expressed HPPD enzymes estimated on the basis of the extent and MIC for inhibition of the formation of the brown, ochronotic pigment.

In another embodiment, candidate nucleic acids are transformed into plant material to generate a transgenic plant, regenerated into morphologically normal fertile plants which are then measured for differential tolerance to selected courmarone-derivative herbicides. Many suitable methods for transformation using suitable selection markers such as kanamycin, binary vectors such as from Agrobacterium and plant regeneration as, for example, from tobacco leaf discs are well known in the art. Optionally, a control population of plants is likewise transformed with a nuclaic acid expressing the control HPPD. Alternatively, an untransformed dicot plant such as Arabidopsis or Tobacco can be used as a control since this, in any case, expresses its own endogenous HPPD. The average, and distribution, of herbicide tolerance levels of a range of primary plant transformation events or their progeny to courmarone-derivative selected from Table 2 are evaluated in the normal manner based upon plant damage, meristematic bleaching symptoms etc. at a range of different concentrations of herbicides. These data can be expressed in terms of, for example, GR50 values derived from dose/response curves having “dose” plotted on the x-axis and “percentage kill”, “herbicidal effect”, “numbers of emerging green plants” etc. plotted on the y-axis where increased GR50 values correspond to increased levels of inherent tolerance of the expressed HPPD. Herbicides can suitably be applied pre-emergence or post-emergence.

Another object refers to an isolated nucleic acid encoding a mut-HPPD, wherein the nucleic acid is identifiable by a method as defined above.

In another embodiment, the invention refers to a plant cell transformed by a wild-type or a mut-HPPD nucleic acid or or a plant cell which has been mutated to obtain a plant expressing a wild-type or a mut-HPPD nucleic acid, wherein expression of the nucleic acid in the plant cell results in increased resistance or tolerance to a coumarone-derivative herbicide as compared to a wild type variety of the plant cell.

The term “expression/expressing” or “gene expression” means the transcription of a specific gene or specific genes or specific genetic construct. The term “expression” or “gene expression” in particular means the transcription of a gene or genes or genetic construct into structural RNA (rRNA, tRNA) or mRNA with or without subsequent translation of the latter into a protein. The process includes transcription of DNA and processing of the resulting mRNA product.

To obtain the desired effect, i.e. plants that are tolerant or resistant to the coumarone-derivative herbicide derivative herbicide of the present invention, it will be understood that the at least one nucleic acid is “over-expressed” by methods and means known to the person skilled in the art.

The term “increased expression” or “overexpression” as used herein means any form of expression that is additional to the original wild-type expression level. Methods for increasing expression of genes or gene products are well documented in the art and include, for example, overexpression driven by appropriate promoters, the use of transcription enhancers or translation enhancers. Isolated nucleic acids which serve as promoter or enhancer elements may be introduced in an appropriate position (typically upstream) of a non-heterologous form of a polynucleotide so as to upregulate expression of a nucleic acid encoding the polypeptide of interest. For example, endogenous promoters may be altered in vivo by mutation, deletion, and/or substitution (see, Kmiec, U.S. Pat. No. 5,565,350; Zarling et al., WO9322443), or isolated promoters may be introduced into a plant cell in the proper orientation and distance from a gene of the present invention so as to control the expression of the gene.

If polypeptide expression is desired, it is generally desirable to include a polyadenylation region at the 3′-end of a polynucleotide coding region. The polyadenylation region can be derived from the natural gene, from a variety of other plant genes, or from T-DNA. The 3′ end sequence to be added may be derived from, for example, the nopaline synthase or octopine synthase genes, or alternatively from another plant gene, or less preferably from any other eukaryotic gene.

An intron sequence may also be added to the 5′ untranslated region (UTR) or the coding sequence of the partial coding sequence to increase the amount of the mature message that accumulates in the cytosol. Inclusion of a spliceable intron in the transcription unit in both plant and animal expression constructs has been shown to increase gene expression at both the mRNA and protein levels up to 1000-fold (Buchman and Berg (1988) Mol. Cell. biol. 8: 4395-4405; Callis et al. (1987) Genes Dev 1:1183-1200). Such intron enhancement of gene expression is typically greatest when placed near the 5′ end of the transcription unit. Use of the maize introns Adh1-5 intron 1, 2, and 6, the Bronze-1 intron are known in the art. For general information see: The Maize Handbook, Chapter 116, Freeling and Walbot, Eds., Springer, N.Y. (1994)

The term “introduction” or “transformation” as referred to herein encompasses the transfer of an exogenous polynucleotide into a host cell, irrespective of the method used for transfer. Plant tissue capable of subsequent clonal propagation, whether by organogenesis or embryogenesis, may be transformed with a genetic construct of the present invention and a whole plant regenerated there from. The particular tissue chosen will vary depending on the clonal propagation systems available for, and best suited to, the particular species being transformed. Exemplary tissue targets include leaf disks, pollen, embryos, cotyledons, hypocotyls, megagametophytes, callus tissue, existing meristematic tissue (e.g., apical meristem, axillary buds, and root meristems), and induced meristem tissue (e.g., cotyledon meristem and hypocotyl meristem). The polynucleotide may be transiently or stably introduced into a host cell and may be maintained non-integrated, for example, as a plasmid. Alternatively, it may be integrated into the host genome. The resulting transformed plant cell may then be used to regenerate a transformed plant in a manner known to persons skilled in the art.

The transfer of foreign genes into the genome of a plant is called transformation. Transformation of plant species is now a fairly routine technique. Advantageously, any of several transformation methods may be used to introduce the gene of interest into a suitable ancestor cell. The methods described for the transformation and regeneration of plants from plant tissues or plant cells may be utilized for transient or for stable transformation. Transformation methods include the use of liposomes, electroporation, chemicals that increase free DNA uptake, injection of the DNA directly into the plant, particle gun bombardment, transformation using viruses or pollen and microprojection. Methods may be selected from the calcium/polyethylene glycol method for protoplasts (Krens, F. A. et al., (1982) Nature 296, 72-74; Negrutiu I et al. (1987) Plant Mol Biol 8: 363-373); electroporation of protoplasts (Shillito R. D. et al. (1985) Bio/Technol 3, 1099-1102); microinjection into plant material (Crossway A et al., (1986) Mol. Gen. Genet. 202: 179-185); DNA or RNA-coated particle bombardment (Klein T M et al., (1987) Nature 327: 70) infection with (non-integrative) viruses and the like. Transgenic plants, including transgenic crop plants, are preferably produced via Agrobacterium-mediated transformation. An advantageous transformation method is the transformation in planta. To this end, it is possible, for example, to allow the agrobacteria to act on plant seeds or to inoculate the plant meristem with agrobacteria. It has proved particularly expedient in accordance with the invention to allow a suspension of transformed agrobacteria to act on the intact plant or at least on the flower primordia. The plant is subsequently grown on until the seeds of the treated plant are obtained (Clough and Bent, Plant J. (1998) 16, 735-743). Methods for Agrobacterium-mediated transformation of rice include well known methods for rice transformation, such as those described in any of the following: European patent application EP 1198985 A1, Aldemita and Hodges (Planta 199: 612-617, 1996); Chan et al. (Plant Mol Biol 22 (3): 491-506, 1993), Hiei et al. (Plant J 6 (2): 271-282, 1994), which disclosures are incorporated by reference herein as if fully set forth. In the case of corn transformation, the preferred method is as described in either Ishida et al. (Nat. Biotechnol 14(6): 745-50, 1996) or Frame et al. (Plant Physiol 129(1): 13-22, 2002), which disclosures are incorporated by reference herein as if fully set forth. Said methods are further described by way of example in B. Jenes et al., Techniques for Gene Transfer, in: Transgenic Plants, Vol. 1, Engineering and Utilization, eds. S. D. Kung and R. Wu, Academic Press (1993) 128-143 and in Potrykus Annu. Rev. Plant Physiol. Plant Molec. Biol. 42 (1991) 205-225). The nucleic acids or the construct to be expressed is preferably cloned into a vector, which is suitable for trans-forming Agrobacterium tumefaciens, for example pBin19 (Bevan et al., Nucl. Acids Res. 12 (1984) 8711). Agrobacteria transformed by such a vector can then be used in known manner for the transformation of plants, such as plants used as a model, like Arabidopsis (Arabidopsis thaliana is within the scope of the present invention not considered as a crop plant), or crop plants such as, by way of example, tobacco plants, for example by immersing bruised leaves or chopped leaves in an agrobacterial solution and then culturing them in suitable media. The transformation of plants by means of Agrobacterium tumefaciens is described, for example, by Höfgen and Willmitzer in Nucl. Acid Res. (1988) 16, 9877 or is known inter alia from F. F. White, Vectors for Gene Transfer in Higher Plants; in Transgenic Plants, Vol. 1, Engineering and Utilization, eds. S. D. Kung and R. Wu, Academic Press, 1993, pp. 15-38.

In addition to the transformation of somatic cells, which then have to be regenerated into intact plants, it is also possible to transform the cells of plant meristems and in particular those cells which develop into gametes. In this case, the transformed gametes follow the natural plant development, giving rise to transgenic plants. Thus, for example, seeds of Arabidopsis are treated with agrobacteria and seeds are obtained from the developing plants of which a certain proportion is transformed and thus transgenic [Feldman, K A and Marks M D (1987). Mol Gen Genet. 208:274-289; Feldmann K (1992). In: C Koncz, N—H Chua and J Shell, eds, Methods in Arabidopsis Research. Word Scientific, Singapore, pp. 274-289]. Alternative methods are based on the repeated removal of the inflorescences and incubation of the excision site in the center of the rosette with transformed agrobacteria, whereby trans-formed seeds can likewise be obtained at a later point in time (Chang (1994). Plant J. 5: 551-558; Katavic (1994). Mol Gen Genet, 245: 363-370). However, an especially effective method is the vacuum infiltration method with its modifications such as the “floral dip” method. In the case of vacuum infiltration of Arabidopsis, intact plants under reduced pressure are treated with an agrobacterial suspension [Bechthold, N (1993). C R Acad Sci Paris Life Sci, 316: 1194-1199], while in the case of the “floral dip” method the developing floral tissue is incubated briefly with a surfactant-treated agrobacterial suspension [Clough, S J and Bent A F (1998) The Plant J. 16, 735-743]. A certain proportion of transgenic seeds are harvested in both cases, and these seeds can be distinguished from non-transgenic seeds by growing under the above-described selective conditions. In addition the stable transformation of plastids is of advantages because plastids are inherited maternally is most crops reducing or eliminating the risk of transgene flow through pollen. The transformation of the chloroplast genome is generally achieved by a process which has been schematically displayed in Klaus et al., 2004 [Nature Biotechnology 22 (2), 225-229]. Briefly the sequences to be transformed are cloned together with a selectable marker gene between flanking sequences homologous to the chloroplast genome. These homologous flanking sequences direct site specific integration into the plastome. Plastidal transformation has been described for many different plant species and an overview is given in Bock (2001) Transgenic plastids in basic research and plant biotechnology. J Mol. Biol. 2001 Sep. 21; 312 (3):425-38 or Maliga, P (2003) Progress towards commercialization of plastid transformation technology. Trends Biotechnol. 21, 20-28. Further biotechnological progress has recently been reported in form of marker free plastid transformants, which can be produced by a transient co-integrated maker gene (Klaus et al., 2004, Nature Biotechnology 22(2), 225-229). The genetically modified plant cells can be regenerated via all methods with which the skilled worker is familiar. Suitable methods can be found in the abovementioned publications by S. D. Kung and R. Wu, Potrykus or Hofgen and Willmitzer.

Generally after transformation, plant cells or cell groupings are selected for the presence of one or more markers which are encoded by plant-expressible genes co-transferred with the gene of interest, following which the transformed material is regenerated into a whole plant. To select transformed plants, the plant material obtained in the transformation is, as a rule, subjected to selective conditions so that transformed plants can be distinguished from untransformed plants. For example, the seeds obtained in the above-described manner can be planted and, after an initial growing period, subjected to a suitable selection by spraying. A further possibility consists in growing the seeds, if appropriate after sterilization, on agar plates using a suitable selection agent so that only the transformed seeds can grow into plants. Alternatively, the transformed plants are screened for the presence of a selectable marker such as the ones described above.

Following DNA transfer and regeneration, putatively transformed plants may also be evaluated, for instance using Southern analysis, for the presence of the gene of interest, copy number and/or genomic organisation. Alternatively or additionally, expression levels of the newly introduced DNA may be monitored using Northern and/or Western analysis, both techniques being well known to persons having ordinary skill in the art.

The generated transformed plants may be propagated by a variety of means, such as by clonal propagation or classical breeding techniques. For example, a first generation (or T1) transformed plant may be selfed and homozygous second-generation (or T2) transformants selected, and the T2 plants may then further be propagated through classical breeding techniques. The generated transformed organisms may take a variety of forms. For example, they may be chimeras of transformed cells and non-transformed cells; clonal transformants (e.g., all cells transformed to contain the expression cassette); grafts of transformed and untrans-formed tissues (e.g., in plants, a transformed rootstock grafted to an untransformed scion).

Preferably, the wild-type or mut-HPPD nucleic acid (a) or wild-type or mut-HST nucleic acid (b) comprises a polynucleotide sequence selected from the group consisting of: a) a polynucleotide as shown in SEQ ID NO: 1, 3 or 5, or a variant or derivative thereof; b) a polynucleotide as shown in SEQ ID NO: 7 or 9, or a variant or derivative thereof; c) a polynucleotide encoding a polypeptide as shown in SEQ ID NO: 2, 4, 6, 8, or 10, or a variant or derivative thereof; d) a polynucleotide comprising at least 60 consecutive nucleotides of any of a) through c); and e) a polynucleotide complementary to the polynucleotide of any of a) through d).

Preferably, the expression of the nucleic acid in the plant results in the plant's increased resistance to coumarone-derivative herbicide as compared to a wild type variety of the plant.

In another embodiment, the invention refers to a plant, preferably a transgenic plant, comprising a plant cell according to the present invention, wherein expression of the nucleic acid in the plant results in the plant's increased resistance to coumarone-derivative herbicide as compared to a wild type variety of the plant.

The plants described herein can be either transgenic crop plants or non-transgenic plants.

For the purposes of the invention, “transgenic”, “transgene” or “recombinant” means with regard to, for example, a nucleic acid sequence, an expression cassette, gene construct or a vector comprising the nucleic acid sequence or an organism transformed with the nucleic acid sequences, expression cassettes or vectors according to the invention, all those constructions brought about by recombinant methods in which either

(a) the nucleic acid sequences encoding proteins useful in the methods of the invention, or
(b) genetic control sequence(s) which is operably linked with the nucleic acid sequence according to the invention, for example a promoter, or
(c) a) and b) are not located in their natural genetic environment or have been modified by recombinant methods, it being possible for the modification to take the form of, for example, a substitution, addition, deletion, inversion or insertion of one or more nucleotide residues. The natural genetic environment is understood as meaning the natural genomic or chromosomal locus in the original plant or the presence in a genomic library. In the case of a genomic library, the natural genetic environment of the nucleic acid sequence is preferably retained, at least in part. The environment flanks the nucleic acid sequence at least on one side and has a sequence length of at least 50 bp, preferably at least 500 bp, especially preferably at least 1000 bp, most preferably at least 5000 bp. A naturally occurring expression cassette—for example the naturally occurring combination of the natural promoter of the nucleic acid sequences with the corresponding nucleic acid sequence encoding a polypeptide useful in the methods of the present invention, as defined above—becomes a transgenic expression cassette when this expression cassette is modified by non-natural, synthetic (“artificial”) methods such as, for example, mutagenic treatment. Suitable methods are described, for example, in U.S. Pat. No. 5,565,350 or WO 00/15815.

A transgenic plant for the purposes of the invention is thus understood as meaning, as above, that the nucleic acids used in the method of the invention are not at their natural locus in the genome of said plant, it being possible for the nucleic acids to be expressed homologously or heterologously. However, as mentioned, transgenic also means that, while the nucleic acids according to the invention or used in the inventive method are at their natural position in the genome of a plant, the sequence has been modified with regard to the natural sequence, and/or that the regulatory sequences of the natural sequences have been modified. Transgenic is preferably understood as meaning the expression of the nucleic acids according to the invention at an unnatural locus in the genome, i.e. homologous or, preferably, heterologous expression of the nucleic acids takes place. Preferred transgenic plants are mentioned herein. Furthermore, the term “transgenic” refers to any plant, plant cell, callus, plant tissue, or plant part, that contains all or part of at least one recombinant polynucleotide. In many cases, all or part of the recombinant polynucleotide is stably integrated into a chromosome or stable extra-chromosomal element, so that it is passed on to successive generations. For the purposes of the invention, the term “recombinant polynucleotide” refers to a polynucleotide that has been altered, rearranged, or modified by genetic engineering. Examples include any cloned polynucleotide, or polynucleotides, that are linked or joined to heterologous sequences. The term “recombinant” does not refer to alterations of polynucleotides that result from naturally occurring events, such as spontaneous mutations, or from non-spontaneous mutagenesis followed by selective breeding.

Plants containing mutations arising due to non-spontaneous mutagenesis and selective breeding are referred to herein as non-transgenic plants and are included in the present invention. In embodiments wherein the plant is transgenic and comprises multiple mut-HPPD nucleic acids, the nucleic acids can be derived from different genomes or from the same genome. Alternatively, in embodiments wherein the plant is non-transgenic and comprises multiple mut-HPPD nucleic acids, the nucleic acids are located on different genomes or on the same genome.

In certain embodiments, the present invention involves herbidicide-resistant plants that are produced by mutation breeding. Such plants comprise a polynucleotide encoding a mut-HPPD and/or a mut-HST and are tolerant to one or more “coumarone-derivative herbicides”. Such methods can involve, for example, exposing the plants or seeds to a mutagen, particularly a chemical mutagen such as, for example, ethyl methanesulfonate (EMS) and selecting for plants that have enhanced tolerance to at least one or more coumarone-derivative herbicide.

However, the present invention is not limited to herbicide-tolerant plants that are produced by a mutagenesis method involving the chemical mutagen EMS. Any mutagenesis method known in the art may be used to produce the herbicide-resistant plants of the present invention. Such mutagenesis methods can involve, for example, the use of any one or more of the following mutagens: radiation, such as X-rays, Gamma rays (e.g., cobalt 60 or cesium 137), neutrons, (e.g., product of nuclear fission by uranium 235 in an atomic reactor), Beta radiation (e.g., emitted from radioisotopes such as phosphorus 32 or carbon 14), and ultraviolet radiation (preferably from 2500 to 2900 nm), and chemical mutagens such as base analogues (e.g., 5-bromo-uracil), related compounds (e.g., 8-ethoxy caffeine), antibiotics (e.g., streptonigrin), alkylating agents (e.g., sulfur mustards, nitrogen mustards, epoxides, ethylenamines, sulfates, sulfonates, sulfones, lactones), azide, hydroxylamine, nitrous acid, or acridines. Herbicide-resistant plants can also be produced by using tissue culture methods to select for plant cells comprising herbicide-resistance mutations and then regenerating herbicide-resistant plants therefrom. See, for example, U.S. Pat. Nos. 5,773,702 and 5,859,348, both of which are herein incorporated in their entirety by reference. Further details of mutation breeding can be found in “Principals of Cultivar Development” Fehr, 1993 Macmillan Publishing Company the disclosure of which is incorporated herein by reference

In addition to the definition above, the term “plant” is intended to encompass crop plants at any stage of maturity or development, as well as any tissues or organs (plant parts) taken or derived from any such plant unless otherwise clearly indicated by context. Plant parts include, but are not limited to, stems, roots, flowers, ovules, stamens, leaves, embryos, meristematic regions, callus tissue, anther cultures, gametophytes, sporophytes, pollen, microspores, protoplasts, and the like.

The plant of the present invention comprises at least one mut-HPPD nucleic acid or over-expressed wild-type HPPD nucleic acid, and has increased tolerance to a coumarone-derivative herbicide as compared to a wild-type variety of the plant. It is possible for the plants of the present invention to have multiple wild-type or mut-HPPD nucleic acids from different genomes since these plants can contain more than one genome. For example, a plant contains two genomes, usually referred to as the A and B genomes. Because HPPD is a required metabolic enzyme, it is assumed that each genome has at least one gene coding for the HPPD enzyme (i.e. at least one HPPD gene). As used herein, the term “HPPD gene locus” refers to the position of an HPPD gene on a genome, and the terms “HPPD gene” and “HPPD nucleic acid” refer to a nucleic acid encoding the HPPD enzyme. The HPPD nucleic acid on each genome differs in its nucleotide sequence from an HPPD nucleic acid on another genome. One of skill in the art can determine the genome of origin of each HPPD nucleic acid through genetic crossing and/or either sequencing methods or exonuclease digestion methods known to those of skill in the art.

The present invention includes plants comprising one, two, three, or more mut-HPPD alleles, wherein the plant has increased tolerance to a coumarone-derivative herbicide as compared to a wild-type variety of the plant. The mut-HPPD alleles can comprise a nucleotide sequence selected from the group consisting of a polynucleotide as defined in SEQ ID NO:1, SEQ ID NO:3, or SEQ ID NO:5, or a variant or derivative thereof, a polynucleotide encoding a polypeptide as defined in SEQ ID NO:2, SEQ ID NO:4, or SEQ ID NOs: 6, 11, 12, 13, 14, 15, 16, 17, 18, 19, or a variant or derivative, homologue, orthologue, paralogue thereof, a polynucleotide comprising at least 60 consecutive nucleotides of any of the aforementioned polynucleotides; and a polynucleotide complementary to any of the aforementioned polynucleotides.

“Alleles” or “allelic variants” are alternative forms of a given gene, located at the same chromosomal position. Allelic variants encompass Single Nucleotide Polymorphisms (SNPs), as well as Small Insertion/Deletion Polymorphisms (INDELs). The size of INDELs is usually less than 100 bp. SNPs and INDELs form the largest set of sequence variants in naturally occurring polymorphic strains of most organisms

The term “variety” refers to a group of plants within a species defined by the sharing of a common set of characteristics or traits accepted by those skilled in the art as sufficient to distinguish one cultivar or variety from another cultivar or variety. There is no implication in either term that all plants of any given cultivar or variety will be genetically identical at either the whole gene or molecular level or that any given plant will be homozygous at all loci. A cultivar or variety is considered “true breeding” for a particular trait if, when the true-breeding cultivar or variety is self-pollinated, all of the progeny contain the trait. The terms “breeding line” or “line” refer to a group of plants within a cultivar defined by the sharing of a common set of characteristics or traits accepted by those skilled in the art as sufficient to distinguish one breeding line or line from another breeding line or line. There is no implication in either term that all plants of any given breeding line or line will be genetically identical at either the whole gene or molecular level or that any given plant will be homozygous at all loci. A breeding line or line is considered “true breeding” for a particular trait if, when the true-breeding line or breeding line is self-pollinated, all of the progeny contain the trait. In the present invention, the trait arises from a mutation in a HPPD gene of the plant or seed.

The herbicide-resistant plants of the invention that comprise polynucleotides encoding mut-HPPD and/or mut-HST polypeptides also find use in methods for increasing the herbicide-resistance of a plant through conventional plant breeding involving sexual reproduction. The methods comprise crossing a first plant that is a herbicide-resistant plant of the invention to a second plant that may or may not be resistant to the same herbicide or herbicides as the first plant or may be resistant to different herbicide or herbicides than the first plant. The second plant can be any plant that is capable of producing viable progeny plants (i.e., seeds) when crossed with the first plant. Typically, but not necessarily, the first and second plants are of the same species. The methods can optionally involve selecting for progeny plants that comprise the mut-HPPD and/or mut-HST polypeptides of the first plant and the herbicide resistance characteristics of the second plant. The progeny plants produced by this method of the present invention have increased resistance to a herbicide when compared to either the first or second plant or both. When the first and second plants are resistant to different herbicides, the progeny plants will have the combined herbicide tolerance characteristics of the first and second plants. The methods of the invention can further involve one or more generations of backcrossing the progeny plants of the first cross to a plant of the same line or genotype as either the first or second plant. Alternatively, the progeny of the first cross or any subsequent cross can be crossed to a third plant that is of a different line or genotype than either the first or second plant. The present invention also provides plants, plant organs, plant tissues, plant cells, seeds, and non-human host cells that are transformed with the at least one polynucleotide molecule, expression cassette, or transformation vector of the invention. Such trans-formed plants, plant organs, plant tissues, plant cells, seeds, and non-human host cells have enhanced tolerance or resistance to at least one herbicide, at levels of the herbicide that kill or inhibit the growth of an untransformed plant, plant tissue, plant cell, or non-human host cell, respectively. Preferably, the transformed plants, plant tissues, plant cells, and seeds of the invention are Arabidopsis thaliana and crop plants.

It is to be understood that the plant of the present invention can comprise a wild type HPPD nucleic acid in addition to a mut-HPPD nucleic acid. It is contemplated that the coumarone-derivative herbicide tolerant lines may contain a mutation in only one of multiple HPPD isoenzymes. Therefore, the present invention includes a plant comprising one or more mut-HPPD nucleic acids in addition to one or more wild type HPPD nucleic acids.

In another embodiment, the invention refers to a seed produced by a transgenic plant comprising a plant cell of the present invention, wherein the seed is true breeding for an increased resistance to a coumarone-derivative herbicide as compared to a wild type variety of the seed.

In another embodiment, the invention refers to a method of producing a transgenic plant cell with an increased resistance to a coumarone-derivative herbicide as compared to a wild type variety of the plant cell comprising, transforming the plant cell with an expression cassette comprising a mut-HPPD nucleic acid.

In another embodiment, the invention refers to a method of producing a transgenic plant comprising, (a) transforming a plant cell with an expression cassette comprising a mut-HPPD nucleic acid, and (b) generating a plant with an increased resistance to coumarone-derivative herbicide from the plant cell.

Consequently, mut-HPPD nucleic acids of the invention are provided in expression cassettes for expression in the plant of interest. The cassette will include regulatory sequences operably linked to a mut-HPPD nucleic acid sequence of the invention. The term “regulatory element” as used herein refers to a polynucleotide that is capable of regulating the transcription of an operably linked polynucleotide. It includes, but not limited to, promoters, enhancers, introns, 5′ UTRs, and 3′ UTRs. By “operably linked” is intended a functional linkage between a promoter and a second sequence, wherein the promoter sequence initiates and mediates transcription of the DNA sequence corresponding to the second sequence. Generally, operably linked means that the nucleic acid sequences being linked are contiguous and, where necessary to join two protein coding regions, contiguous and in the same reading frame. The cassette may additionally contain at least one additional gene to be cotransformed into the organism. Alternatively, the additional gene(s) can be provided on multiple expression cassettes.

Such an expression cassette is provided with a plurality of restriction sites for insertion of the mut-HPPD nucleic acid sequence to be under the transcriptional regulation of the regulatory regions. The expression cassette may additionally contain selectable marker genes.

The expression cassette will include in the 5′-3′ direction of transcription, a transcriptional and translational initiation region (i.e., a promoter), a mut-HPPD nucleic acid sequence of the invention, and a transcriptional and translational termination region (i.e., termination region) functional in plants. The promoter may be native or analogous, or foreign or heterologous, to the plant host and/or to the mut-HPPD nucleic acid sequence of the invention. Additionally, the promoter may be the natural sequence or alternatively a synthetic sequence. Where the promoter is “foreign” or “heterologous” to the plant host, it is intended that the promoter is not found in the native plant into which the promoter is introduced. Where the promoter is “foreign” or “heterologous” to the mut-HPPD nucleic acid sequence of the invention, it is intended that the promoter is not the native or naturally occurring promoter for the operably linked mut-HPPD nucleic acid sequence of the invention. As used herein, a chimeric gene comprises a coding sequence operably linked to a transcription initiation region that is heterologous to the coding sequence.

While it may be preferable to express the mut-HPPD nucleic acids of the invention using heterologous promoters, the native promoter sequences may be used. Such constructs would change expression levels of the mut-HPPD protein in the plant or plant cell. Thus, the phenotype of the plant or plant cell is altered.

The termination region may be native with the transcriptional initiation region, may be native with the operably linked mut-HPPD sequence of interest, may be native with the plant host, or may be derived from another source (i.e., foreign or heterologous to the promoter, the mut-HPPD nucleic acid sequence of interest, the plant host, or any combination thereof). Convenient termination regions are available from the Ti-plasmid of A. tumefaciens, such as the octopine synthase and nopaline synthase termination regions. See also Guerineau et al. (1991) Mol. Gen. Genet. 262: 141-144; Proudfoot (1991) Cell 64:671-674; Sanfacon et al. (1991) Genes Dev. 5: 141-149; Mogen et al. (1990) Plant Cell 2: 1261-1272; Munroe et al. (1990) Gene 91: 151-158; Ballast al. (1989) Nucleic Acids Res. 17:7891-7903; and Joshi ̂[alpha]/. (1987) Nucleic Acid Res. 15:9627-9639. Where appropriate, the gene(s) may be optimized for increased expression in the transformed plant. That is, the genes can be synthesized using plant-preferred codons for improved expression. See, for example, Campbell and Gowri (1990) Plant Physiol. 92: 1-11 for a discussion of host-preferred codon usage. Methods are available in the art for synthesizing plant-preferred genes. See, for example, U.S. Pat. Nos. 5,380,831, and 5,436,391, and Murray et al. (1989) Nucleic Acids Res. 17:477-498, herein incorporated by reference.

Additional sequence modifications are known to enhance gene expression in a cellular host. These include elimination of sequences encoding spurious polyadenylation signals, exonintron splice site signals, transposon-like repeats, and other such well-characterized sequences that may be deleterious to gene expression. The G-C content of the sequence may be adjusted to levels average for a given cellular host, as calculated by reference to known genes expressed in the host cell. When possible, the sequence is modified to avoid predicted hairpin secondary mRNA structures. Nucleotide sequences for enhancing gene expression can also be used in the plant expression vectors. These include the introns of the maize Adhl, intronl gene (Callis et al. Genes and Development 1: 1183-1200, 1987), and leader sequences, (W-sequence) from the Tobacco Mosaic virus (TMV), Maize Chlorotic Mottle Virus and Alfalfa Mosaic Virus (Gallie et al. Nucleic Acid Res. 15:8693-8711, 1987 and Skuzeski et al. Plant Mol. Biol. 15:65-79, 1990). The first intron from the shrunken-1 locus of maize, has been shown to increase expression of genes in chimeric gene constructs. U.S. Pat. Nos. 5,424,412 and 5,593,874 disclose the use of specific introns in gene expression constructs, and Gallie et al. (Plant Physiol. 106:929-939, 1994) also have shown that introns are useful for regulating gene expression on a tissue specific basis. To further enhance or to optimize mut-HPPD gene expression, the plant expression vectors of the invention may also contain DNA sequences containing matrix attachment regions (MARs). Plant cells transformed with such modified expression systems, then, may exhibit overexpression or constitutive expression of a nucleotide sequence of the invention.

The expression cassettes may additionally contain 5′ leader sequences in the expression cassette construct. Such leader sequences can act to enhance translation. Translation leaders are known in the art and include: picornavirus leaders, for example, EMCV leader (Encephalomyocarditis 5′ noncoding region) (Elroy-Stein et al. (1989) Proc. Natl. Acad. ScL USA 86:6126-6130); potyvirus leaders, for example, TEV leader (Tobacco Etch Virus) (Gallie et al. (1995) Gene 165(2):233-238), MDMV leader (Maize Dwarf Mosaic Virus) (Virology 154:9-20), and human immunoglobulin heavy-chain binding protein (BiP) (Macejak et al. (1991) Nature 353:90-94); untranslated leader from the coat protein mRNA of alfalfa mosaic virus (AMV RNA 4) (Jobling et al. (1987) Nature 325:622-625); tobacco mosaic virus leader (TMV) (Gallie et al. (1989) in Molecular Biology of RNA, ed. Cech (Liss, New York), pp. 237-256); and maize chlorotic mottle virus leader (MCMV) (Lommel et al. (1991) Virology 81:382-385). See also, Della-Cioppa et al. (1987) Plant Physiol. 84:965-968. Other methods known to enhance translation can also be utilized, for example, introns, and the like.

In preparing the expression cassette, the various DNA fragments may be manipulated, so as to provide for the DNA sequences in the proper orientation and, as appropriate, in the proper reading frame. Toward this end, adapters or linkers may be employed to join the DNA fragments or other manipulations may be involved to provide for convenient restriction sites, removal of superfluous DNA, removal of restriction sites, or the like. For this purpose, in vitro mutagenesis, primer repair, restriction, annealing, resubstitutions, e.g., transitions and trans versions, may be involved.

A number of promoters can be used in the practice of the invention. The promoters can be selected based on the desired outcome. The nucleic acids can be combined with constitutive, tissue-preferred, or other promoters for expression in plants. Such constitutive promoters include, for example, the core promoter of the Rsyn7 promoter and other constitutive promoters disclosed in WO 99/43838 and U.S. Pat. No. 6,072,050; the core CaMV 35S promoter (Odell et al. (1985) Nature 313:810-812); rice actin (McElroy et al. (1990) Plant Cell 2: 163-171); ubiquitin (Christensen et al. (1989) Plant Mol. Biol. 12:619-632 and Christensen et al. (1992) Plant Mol. Biol. 18:675-689); pEMU (Last et al. (1991) Theor. Appl. Genet. 81:581-588); MAS (Velten et al. (1984) EMBO J. 3:2723-2730); ALS promoter (U.S. Pat. No. 5,659,026), and the like. Other constitutive promoters include, for example, U.S. Pat. Nos. 5,608,149; 5,608,144; 5,604,121; 5,569,597; 5,466,785; 5,399,680; 5,268,463; 5,608,142; and 6,177,611.

Tissue-preferred promoters can be utilized to target enhanced mut-HPPD expression within a particular plant tissue. Such tissue-preferred promoters include, but are not limited to, leaf preferred promoters, root-preferred promoters, seed-preferred promoters, and stem-preferred promoters. Tissue-preferred promoters include Yamamoto et al. (1997) Plant J. 12(2):255-265; Kawamata et al. (1997) Plant Cell Physiol. 38(7):792-803; Hansen et al. (1997) Mol. Gen. Genet. 254(3):337-343; Russell et al. (1997) Transgenic Res. 6(2): 157-168; Rinehart et al. (1996) Plant Physiol. 112(3): 1331-1341; Van Camp et al. (1996) Plant Physiol. 112(2):525-535; Canevascini et al. (1996) Plant Physiol. 112(2):513-524; Yamamoto et al. (1994) Plant Cell Physiol. 35(5):773-778; Lam (1994) Results Probl. Cell Differ. 20: 181-196; Orozco et al. (1993) Plant Mol. Biol. 23(6): 1129-1138; Matsuoka e/ [alpha]/. (1993) Proc Natl. Acad. Sci. USA 90(20):9586-9590; and Guevara-Garcia et al. (1993) Plant J. 4(3):495-505. Such promoters can be modified, if necessary, for weak expression. In one embodiment, the nucleic acids of interest are targeted to the chloroplast for expression. In this manner, where the nucleic acid of interest is not directly inserted into the chloroplast, the expression cassette will additionally contain a chloroplast-targeting sequence comprising a nucleotide sequence that encodes a chloroplast transit peptide to direct the gene product of interest to the chloroplasts. Such transit peptides are known in the art. With respect to chloroplast-targeting sequences, “operably linked” means that the nucleic acid sequence encoding a transit peptide (i.e., the chloroplast-targeting sequence) is linked to the mut-HPPD nucleic acid of the invention such that the two sequences are contiguous and in the same reading frame. See, for example, Von Heijne et al. (1991) Plant Mol. Biol. Rep. 9: 104-126; Clark et al. (1989) J. Biol. Chem. 264:17544-17550; Della-Cioppa et al. (1987) Plant Physiol. 84:965-968; Romer et al. (1993) Biochem. Biophys. Res. Commun. 196:1414-1421; and Shah et al. (1986) Science 233:478-481. Any chloroplast transit peptide known in the art can be fused to the amino acid sequence of a mature mut-HPPD protein of the invention by operably linking a choloroplast-targeting sequence to the 5′-end of a nucleotide sequence encoding a mature mut-HPPD protein of the invention. Chloroplast targeting sequences are known in the art and include the chloroplast small subunit of ribulose-1,5-bisphosphate carboxylase (Rubisco) (de Castro Silva Filho et al. (1996) Plant Mol. Biol. 30:769-780; Schnell et al. (1991) J. Biol. Chem. 266(5):3335-3342); 5-(enolpyruvyl)shikimate-3-phosphate synthase (EPSPS) (Archer et al. (1990) J. Bioenerg. Biomemb. 22(6):789-810); tryptophan synthase (Zhao et al. (1995) J. Biol. Chem. 270(11):6081-6087); plastocyanin (Lawrence et al. (1997) J. Biol. Chem. 272(33):20357-20363); chorismate synthase (Schmidt et al. (1993) J. Biol. Chem. 268(36):27447-27457); and the light harvesting chlorophyll a/b binding protein (LHBP) (Lamppa et al. (1988) J. Biol. Chem. 263: 14996-14999). See also Von Heijne et al. (1991) Plant Mol. Biol. Rep. 9: 104-126; Clark et al. (1989) J. Biol. Chem. 264:17544-17550; Della-Cioppa et al. (1987) Plant Physiol. 84:965-968; Romer et al. (1993) Biochem. Biophys. Res. Commun. 196: 1414-1421; and Shah et al. (1986) Science 233:478-481.

Methods for transformation of chloroplasts are known in the art. See, for example, Svab et al. (1990) Proc. Natl. Acad. ScL USA 87:8526-8530; Svab and Maliga (1993) Proc. Natl. Acad. Sci. USA 90:913-917; Svab and Maliga (1993) EMBO J. 12:601-606. The method relies on particle gun delivery of DNA containing a selectable marker and targeting of the DNA to the plastid genome through homologous recombination. Additionally, plastid transformation can be accomplished by transactivation of a silent plastid-borne transgene by tissue-preferred expression of a nuclear-encoded and plastid-directed RNA polymerase. Such a system has been reported in McBride et al. (1994) Proc. Natl. Acad. Sci. USA 91:7301-7305. The nucleic acids of interest to be targeted to the chloroplast may be optimized for expression in the chloroplast to account for differences in codon usage between the plant nucleus and this organelle. In this manner, the nucleic acids of interest may be synthesized using chloroplastpreferred codons. See, for example, U.S. Pat. No. 5,380,831, herein incorporated by reference.

In a preferred embodiment, the mut-HPPD nucleic acid (a) or the mut-HST nucleic acid (b) comprises a polynucleotide sequence selected from the group consisting of: a) a polynucleotide as shown in SEQ ID NO: 1, 3 or 5, or a variant or derivative thereof; b) a polynucleotide as shown in SEQ ID NO: 7 or 9, or a variant or derivative thereof; c) a polynucleotide encoding a polypeptide as shown in SEQ ID NO: 2, 4, 6, 8, or 10, or a variant or derivative thereof; d) a polynucleotide comprising at least 60 consecutive nucleotides of any of a) through c); and e) a polynucleotide complementary to the polynucleotide of any of a) through d)

Preferably, the expression cassette further comprises a transcription initiation regulatory region and a translation initiation regulatory region that are functional in the plant.

While the polynucleotides of the invention find use as selectable marker genes for plant transformation, the expression cassettes of the invention can include another selectable marker gene for the selection of transformed cells. Selectable marker genes, including those of the present invention, are utilized for the selection of transformed cells or tissues. Marker genes include, but are not limited to, genes encoding antibiotic resistance, such as those encoding neomycin phosphotransferase II (NEO) and hygromycin phosphotransferase (HPT), as well as genes conferring resistance to herbicidal compounds, such as glufosinate ammonium, bromoxynil, imidazolinones, and 2,4-dichlorophenoxyacetate (2,4-D). See generally, Yarranton (1992) Curr. Opin. Biotech. 3:506-511; Christophers on et al (1992) Proc. Natl. Acad. ScL USA 89:6314-6318; Yao et al. (1992) Cell 71:63-72; Reznikoff (1992) Mol Microbiol 6:2419-2422; Barkley et al (1980) in The Operon, pp. 177-220; Hu et al (1987) Cell 48:555-566; Brown et al (1987) Cell 49:603-612; Figge et al (1988) Cell 52:713-722; Deuschle et al (1989) Proc. Natl. Acad. AcL USA 86:5400-5404; Fuerst et al (1989) Proc. Natl. Acad. ScL USA 86:2549-2553; Deuschle et al (1990) Science 248:480-483; Gossen (1993) Ph.D. Thesis, University of Heidelberg; Reines et al (1993) Proc. Natl. Acad. ScL USA 90: 1917-1921; Labow et al (1990) Mol Cell Biol 10:3343-3356; Zambretti et al (1992) Proc. Natl. Acad. ScL USA 89:3952-3956; Bairn et al (1991) Proc. Natl. Acad. ScL USA 88:5072-5076; Wyborski et al (1991) Nucleic Acids Res. 19:4647-4653; Hillenand-Wissman (1989) Topics Mol. Struc. Biol 10: 143-162; Degenkolb et al (1991) Antimicrob. Agents Chemother. 35: 1591-1595; Kleinschnidt et al (1988) Biochemistry 27: 1094-1104; Bonin (1993) Ph.D. Thesis, University of Heidelberg; Gossen et al (1992) Proc. Natl. Acad. ScL USA 89:5547-5551; Oliva et al (1992) Antimicrob. Agents Chemother. 36:913-919; Hlavka et al (1985) Handbook of Experimental Pharmacology, Vol. 78 (Springer-Verlag, Berlin); Gill et al (1988) Nature 334:721-724. Such disclosures are herein incorporated by reference. The above list of selectable marker genes is not meant to be limiting. Any selectable marker gene can be used in the present invention.

The invention further provides an isolated recombinant expression vector comprising the expression cassette containing a mut-HPPD nucleic acid as described above, wherein expression of the vector in a host cell results in increased tolerance to a coumarone-derivative herbicide as compared to a wild type variety of the host cell. As used herein, the term “vector” refers to a nucleic acid molecule capable of transporting another nucleic acid to which it has been linked. One type of vector is a “plasmid,” which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “expression vectors.” In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses, and adeno-associated viruses), which serve equivalent functions.

The recombinant expression vectors of the invention comprise a nucleic acid of the invention in a form suitable for expression of the nucleic acid in a host cell, which means that the recombinant expression vectors include one or more regulatory sequences, selected on the basis of the host cells to be used for expression, which is operably linked to the nucleic acid sequence to be expressed. Regulatory sequences include those that direct constitutive expression of a nucleotide sequence in many types of host cells and those that direct expression of the nucleotide sequence only in certain host cells or under certain conditions. It will be appreciated by those skilled in the art that the design of the expression vector can depend on such factors as the choice of the host cell to be transformed, the level of expression of polypeptide desired, etc. The expression vectors of the invention can be introduced into host cells to thereby produce polypeptides or peptides, including fusion polypeptides or peptides, encoded by nucleic acids as described herein (e.g., mut-HPPD polypeptides, fusion polypeptides, etc.).

In a preferred embodiment of the present invention, the mut-HPPD polypeptides are expressed in plants and plants cells such as unicellular plant cells (such as algae) (See Falciatore et al., 1999, Marine Biotechnology 1(3):239-251 and references therein) and plant cells from higher plants (e.g., the spermatophytes, such as crop plants). A mut-HPPD polynucleotide may be “introduced” into a plant cell by any means, including transfection, transformation or transduction, electroporation, particle bombardment, agroinfection, biolistics, and the like.

Suitable methods for transforming or transfecting host cells including plant cells can be found in Sambrook et al. (Molecular Cloning: A Laboratory Manual. 2nd, ed., Cold Spring Harbor Laboratory, Cold Spring Harbor Laboratory Press, Cold Spring Harbor, N.Y., 1989) and other laboratory manuals such as Methods in Molecular Biology, 1995, Vol. 44, Agrobacterium protocols, ed: Gartland and Davey, Humana Press, Totowa, N.J. As increased tolerance to coumarone-derivative herbicides is a general trait wished to be inherited into a wide variety of plants like maize, wheat, rye, oat, triticale, rice, barley, soybean, peanut, cotton, rapeseed and canola, manihot, pepper, sunflower and tagetes, solanaceous plants like potato, tobacco, eggplant, and tomato, Vicia species, pea, alfalfa, bushy plants (coffee, cacao, tea), Salix species, trees (oil palm, coconut), perennial grasses, and forage crops, these crop plants are also preferred target plants for a genetic engineering as one further embodiment of the present invention. In a preferred embodiment, the plant is a crop plant. Forage crops include, but are not limited to, Wheatgrass, Canarygrass, Bromegrass, Wildrye Grass, Bluegrass, Orchardgrass, Alfalfa, Salfoin, Birdsfoot Trefoil, Alsike Clover, Red Clover, and Sweet Clover.

In one embodiment of the present invention, transfection of a mut-HPPD polynucleotide into a plant is achieved by Agrobacterium mediated gene transfer. One transformation method known to those of skill in the art is the dipping of a flowering plant into an Agrobacteria solution, wherein the Agrobacteria contains the mut-HPPD nucleic acid, followed by breeding of the transformed gametes. Agrobacterium mediated plant transformation can be performed using for example the GV3101(pMP90) (Koncz and Schell, 1986, Mol. Gen. Genet. 204:383-396) or LBA4404 (Clontech) Agrobacterium tumefaciens strain. Transformation can be performed by standard transformation and regeneration techniques (Deblaere et al., 1994, Nucl. Acids. Res. 13:4777-4788; Gelvin, Stanton B. and Schilperoort, Robert A, Plant Molecular Biology Manual, 2nd Ed.-Dordrecht: Kluwer Academic Publ., 1995.—in Sect., Ringbuc Zentrale Signatur: BT11-P ISBN 0-7923-2731-4; Glick, Bernard R. and Thompson, John E., Methods in Plant Molecular Biology and Biotechnology, Boca Raton: CRC Press, 1993 360 S., ISBN 0-8493-5164-2). For example, rapeseed can be transformed via cotyledon or hypocotyl transformation (Moloney et al., 1989, Plant Cell Report 8:238-242; De Block et al., 1989, Plant Physiol. 91:694-701). Use of antibiotics for Agrobacterium and plant selection depends on the binary vector and the Agrobacterium strain used for transformation. Rapeseed selection is normally performed using kanamycin as selectable plant marker. Agrobacterium mediated gene transfer to flax can be performed using, for example, a technique described by Mlynarova et al., 1994, Plant Cell Report 13:282-285. Additionally, transformation of soybean can be performed using for example a technique described in European Patent No. 0424 047, U.S. Pat. No. 5,322,783, European Patent No. 0397 687, U.S. Pat. No. 5,376,543, or U.S. Pat. No. 5,169,770. Transformation of maize can be achieved by particle bombardment, polyethylene glycol mediated DNA uptake, or via the silicon carbide fiber technique. (See, for example, Freeling and Walbot “The maize handbook” Springer Verlag: New York (1993) ISBN 3-540-97826-7). A specific example of maize transformation is found in U.S. Pat. No. 5,990,387, and a specific example of wheat transformation can be found in PCT Application No. WO 93/07256.

According to the present invention, the introduced mut-HPPD polynucleotide may be maintained in the plant cell stably if it is incorporated into a non-chromosomal autonomous replicon or integrated into the plant chromosomes. Alternatively, the introduced mut-HPPD polynucleotide may be present on an extra-chromosomal non-replicating vector and be transiently expressed or transiently active. In one embodiment, a homologous recombinant microorganism can be created wherein the mut-HPPD polynucleotide is integrated into a chromosome, a vector is prepared which contains at least a portion of an HPPD gene into which a deletion, addition, or substitution has been introduced to thereby alter, e.g., functionally disrupt, the endogenous HPPD gene and to create a mut-HPPD gene. To create a point mutation via homologous recombination, DNA-RNA hybrids can be used in a technique known as chimeraplasty (Cole-Strauss et al., 1999, Nucleic Acids Research 27(5):1323-1330 and Kmiec, 1999, Gene therapy American Scientist 87(3):240-247). Other homologous recombination procedures in Triticum species are also well known in the art and are contemplated for use herein.

In the homologous recombination vector, the mut-HPPD gene can be flanked at its 5′ and 3′ ends by an additional nucleic acid molecule of the HPPD gene to allow for homologous recombination to occur between the exogenous mut-HPPD gene carried by the vector and an endogenous HPPD gene, in a microorganism or plant. The additional flanking HPPD nucleic acid molecule is of sufficient length for successful homologous recombination with the endogenous gene. Typically, several hundreds of base pairs up to kilobases of flanking DNA (both at the 5′ and 3′ ends) are included in the vector (see e.g., Thomas, K. R., and Capecchi, M. R., 1987, Cell 51:503 for a description of homologous recombination vectors or Strepp et al., 1998, PNAS, 95(8):4368-4373 for cDNA based recombination in Physcomitrella patens). However, since the mut-HPPD gene normally differs from the HPPD gene at very few amino acids, a flanking sequence is not always necessary. The homologous recombination vector is introduced into a microorganism or plant cell (e.g., via polyethylene glycol mediated DNA), and cells in which the introduced mut-HPPD gene has homologously recombined with the endogenous HPPD gene are selected using art-known techniques.

In another embodiment, recombinant microorganisms can be produced that contain selected systems that allow for regulated expression of the introduced gene. For example, inclusion of a mut-HPPD gene on a vector placing it under control of the lac operon permits expression of the mut-HPPD gene only in the presence of IPTG. Such regulatory systems are well known in the art.

Another aspect of the invention pertains to host cells into which a recombinant expression vector of the invention has been introduced. The terms “host cell” and “recombinant host cell” are used interchangeably herein. It is understood that such terms refer not only to the particular subject cell but they also apply to the progeny or potential progeny of such a cell. Because certain modifications may occur in succeeding generations due to either mutation or environmental influences, such progeny may not, in fact, be identical to the parent cell, but are still included within the scope of the term as used herein. A host cell can be any prokaryotic or eukaryotic cell. For example, a mut-HPPD polynucleotide can be expressed in bacterial cells such as C. glutamicum, insect cells, fungal cells, or mammalian cells (such as Chinese hamster ovary cells (CHO) or COS cells), algae, ciliates, plant cells, fungi or other microorganisms like C. glutamicum. Other suitable host cells are known to those skilled in the art.

A host cell of the invention, such as a prokaryotic or eukaryotic host cell in culture, can be used to produce (i.e., express) a mut-HPPD polynucleotide. Accordingly, the invention further provides methods for producing mut-HPPD polypeptides using the host cells of the invention. In one embodiment, the method comprises culturing the host cell of invention (into which a recombinant expression vector encoding a mut-HPPD polypeptide has been introduced, or into which genome has been introduced a gene encoding a wild-type or mut-HPPD polypeptide) in a suitable medium until mut-HPPD polypeptide is produced. In another embodiment, the method further comprises isolating mut-HPPD polypeptides from the medium or the host cell. Another aspect of the invention pertains to isolated mut-HPPD polypeptides, and biologically active portions thereof. An “isolated” or “purified” polypeptide or biologically active portion thereof is free of some of the cellular material when produced by recombinant DNA techniques, or chemical precursors or other chemicals when chemically synthesized. The language “substantially free of cellular material” includes preparations of mut-HPPD polypeptide in which the polypeptide is separated from some of the cellular components of the cells in which it is naturally or recombinantly produced. In one embodiment, the language “substantially free of cellular material” includes preparations of a mut-HPPD polypeptide having less than about 30% (by dry weight) of non-mut-HPPD material (also referred to herein as a “contaminating polypeptide”), more preferably less than about 20% of non-mut-HPPD material, still more preferably less than about 10% of non-mut-HPPD material, and most preferably less than about 5% non-mut-HPPD material.

When the mut-HPPD polypeptide, or biologically active portion thereof, is recombinantly produced, it is also preferably substantially free of culture medium, i.e., culture medium represents less than about 20%, more preferably less than about 10%, and most preferably less than about 5% of the volume of the polypeptide preparation. The language “substantially free of chemical precursors or other chemicals” includes preparations of mut-HPPD polypeptide in which the polypeptide is separated from chemical precursors or other chemicals that are involved in the synthesis of the polypeptide. In one embodiment, the language “substantially free of chemical precursors or other chemicals” includes preparations of a mut-HPPD polypeptide having less than about 30% (by dry weight) of chemical precursors or non-mut-HPPD chemicals, more preferably less than about 20% chemical precursors or non-mut-HPPD chemicals, still more preferably less than about 10% chemical precursors or non-mut-HPPD chemicals, and most preferably less than about 5% chemical precursors or non-mut-HPPD chemicals. In preferred embodiments, isolated polypeptides, or biologically active portions thereof, lack contaminating polypeptides from the same organism from which the mut-HPPD polypeptide is derived. Typically, such polypeptides are produced by recombinant expression of, for example, a mut-HPPD polypeptide in plants other than, or in microorganisms such as C. glutamicum, ciliates, algae, or fungi.

As described above, the present invention teaches compositions and methods for increasing the coumarone-derivative tolerance of a crop plant or seed as compared to a wild-type variety of the plant or seed. In a preferred embodiment, the coumarone-derivative tolerance of a crop plant or seed is increased such that the plant or seed can withstand a coumarone-derivative herbicide application of preferably approximately 1-1000 g ai ha⁻¹, more preferably 20-160 g ai ha⁻¹, and most preferably 40-80 g ai ha⁻¹. As used herein, to “withstand” a coumarone-derivative herbicide application means that the plant is either not killed or not injured by such application.

Furthermore, the present invention provides methods that involve the use of at least one coumarone-derivative herbicide as depicted in Table 2.

In these methods, the coumarone-derivative herbicide can be applied by any method known in the art including, but not limited to, seed treatment, soil treatment, and foliar treatment. Prior to application, the coumarone-derivative herbicide can be converted into the customary formulations, for example solutions, emulsions, suspensions, dusts, powders, pastes and granules. The use form depends on the particular intended purpose; in each case, it should ensure a fine and even distribution of the compound according to the invention.

By providing plants having increased tolerance to coumarone-derivative herbicide, a wide variety of formulations can be employed for protecting plants from weeds, so as to enhance plant growth and reduce competition for nutrients. A coumarone-derivative herbicide can be used by itself for pre-emergence, post-emergence, pre-planting, and at-planting control of weeds in areas surrounding the crop plants described herein, or a coumarone-derivative herbicide formulation can be used that contains other additives. The coumarone-derivative herbicide can also be used as a seed treatment. Additives found in a coumarone-derivative herbicide formulation include other herbicides, detergents, adjuvants, spreading agents, sticking agents, stabilizing agents, or the like. The coumarone-derivative herbicide formulation can be a wet or dry preparation and can include, but is not limited to, flowable powders, emulsifiable concentrates, and liquid concentrates. The coumarone-derivative herbicide and herbicide formulations can be applied in accordance with conventional methods, for example, by spraying, irrigation, dusting, or the like.

Suitable formulations are describe in detail in PCT/EP2009/063387 and PCT/EP2009/063386, which are incorporated herein by reference.

It should also be understood that the foregoing relates to preferred embodiments of the present invention and that numerous changes may be made therein without departing from the scope of the invention. The invention is further illustrated by the following examples, which are not to be construed in any way as imposing limitations upon the scope thereof. On the contrary, it is to be clearly understood that resort may be had to various other embodiments, modifications, and equivalents thereof, which, after reading the description herein, may suggest themselves to those skilled in the art without departing from the spirit of the present invention and/or the scope of the appended claims.

### EXAMPLES

### Example 1

### Cloning of HPPD Encoding Genes

### (A) Cloning of Arabidopsis Thaliana HPPD

The partial Arabidopsis thaliana AtHPPD coding sequence (SEQ ID No: 1) is amplified by standard PCR techniques from Arabidopsis thaliana cDNA using primers HuJ101 and HuJ102 (Table 5).

TABLE 5
PCR primers for AtHPPD amplification
(SEQ ID NOs: 20, 21)

| Primer | Primer sequence |
|---|---|
| name | (5′ → 3′) |
| HuJ101 | GGCCACCAAAACGCCG |
| HuJ102 | TCATCCCACTAACTGTTTGGCTTC |

The PCR-product is cloned in vector pEXP5-NT/TOPO® (Invitrogen, Carlsbad, USA) according to the manufacturer's instructions. The resulting plasmid pEXP5-NT/TOPO®-AtHPPD is isolated from E. coli TOP10 by performing a plasmid minipreparation. The expression cassette encoding N-terminally Hiss-tagged AtHPPD is confirmed by DNA sequencing.

### (B) Cloning of Chlamydomonas Reinhardtii HPPD1

The C. reinhardtii HPPD1 (CrHPPD1) coding sequence (SEQ ID No: 3) is codon-optimized for expression in E. coli and provided as a synthetic gene (Entelechon, Regensburg, Germany). The partial synthetic gene is amplified by standard PCR techniques using primers Ta1-1 and Ta1-2 (Table 6).

TABLE 6
PCR primers for CrHPPD1 amplification
(SEQ ID NOs: 22, 23)

| Primer | Primer sequence |
|---|---|
| name | (5′ → 3′) |
| Ta1-1 | GGCGCTGGCGGTGCGTCCACTAC |
| Ta1-2 | TCAAACGTTCAGGGTACGCTCGTAGTCTTCGATG |

The PCR-product is cloned in vector pEXP5-NT/TOPO® (Invitrogen, Carlsbad, USA) according to the manufacturer's instructions. The resulting plasmid pEXP5-NT/TOPO®-CrHPPD1 is isolated from E. coli TOP10 by performing a plasmid minipreparation. The expression cassette encoding N-terminally His6-tagged CrHPPD1 is confirmed by DNA sequencing.

### (C) Cloning of C. Reinhardtii HPPD2

The C. reinhardtii HPPD2 (CrHPPD2) coding sequence (SEQ ID No: 5) is codon-optimized for expression in E. coli and provided as a synthetic gene (Entelechon, Regensburg, Germany). The partial synthetic gene is amplified by standard PCR techniques using primers Ta1-3 and Ta1-4 (Table 7).

TABLE 7
PCR primers for CrHPPD2 amplification
(SEQ ID NOs: 24, 25)

| Primer | Primer sequence |
|---|---|
| name | (5′ → 3′) |
| Ta1-3 | GGTGCGGGTGGCGCTGGCACC |
| Ta1-4 | TCAAACGTTCAGGGTACGTTCGTAGTCCTCGATGG |

The PCR-product is cloned in vector pEXP5-NT/TOPO® (Invitrogen, Carlsbad, USA) according to the manufacturer's instructions. The resulting plasmid pEXP5-NT/TOPO®-CrHPPD2 is isolated from E. coli TOP10 by performing a plasmid minipreparation. The expression cassette encoding N-terminally His6-tagged CrHPPD2 is confirmed by DNA sequencing.

### (D) Cloning of Glycine Max HPPD

The Glycine max HPPD (GmHPPD; Glyma14g03410) coding sequence is codon-optimized for expression in E. coli and provided as a synthetic gene (Entelechon, Regensburg, Germany). The partial synthetic gene is amplified by standard PCR techniques using primers Ta2-65 and Ta2-66 (Table 8).

TABLE 8
PCR primers for GmHPPD amplification
(SEQ ID NOs: 26, 27)

| Primer | Primer sequence |
|---|---|
| name | (5′ → 3′) |
| Ta2-65 | CCAATCCCAATGTGCAACG |
| Ta2-66 | TTATGCGGTACGTTTAGCCTCC |

The PCR-product is cloned in vector pEXP5-NT/TOPO® (Invitrogen, Carlsbad, USA) according to the manufacturer's instructions. The resulting plasmid pEXP5-NT/TOPO®-GmHPPD is isolated from E. coli TOP10 by performing a plasmid minipreparation. The expression cassette encoding N-terminally His6-tagged GmHPPD is confirmed by DNA sequencing.

### (E) Cloning of Zea Mays HPPD

The Zea mays HPPD (ZmHPPD; GRMZM2G088396) coding sequence is codon-optimized for expression in E. coli and provided as a synthetic gene (Entelechon, Regensburg, Germany). The partial synthetic gene is amplified by standard PCR techniques using primers Ta2-45 and Ta2-46 (Table 9).

TABLE 9
PCR primer for ZmHPPD amplification
(SEQ ID NOs: 28, 29)

| Primer | Primer sequence |
|---|---|
| name | (5′ → 3′) |
| Ta2-45 | CCACCGACTCCGACCGCCGCAGC |
| Ta2-46 | TCAGGAACCCTGTGCAGCTGCCGCAG |

The PCR-product is cloned in vector pEXP5-NT/TOPO® (Invitrogen, Carlsbad, USA) according to the manufacturer's instructions. The resulting plasmid pEXP5-NT/TOPO®-ZmHPPD is isolated from E. coli TOP10 by performing a plasmid minipreparation. The expression cassette encoding N-terminally His6-tagged ZmHPPD is confirmed by DNA sequencing.

### (F) Cloning of Oryza Sativa HPPD

The Oryza sativa HPPD (OsHPPD; Os02g07160) coding sequence is codon-optimized for expression in E. coli and provided as a synthetic gene (Entelechon, Regensburg, Germany). The partial synthetic gene is amplified by standard PCR techniques using primers Ta2-63 and Ta2-64 (Table 10).

TABLE 10
PCR primer for OsHPPD amplification
(SEQ ID NOs: 30, 31)

| Primer | Primer sequence |
|---|---|
| name | (5′ → 3′) |
| Ta2-63 | CCGCCGACTCCAACCCC |
| Ta2-64 | TTAAGAACCCTGAACGGTCGG |

The PCR-product is cloned in vector pEXP5-NT/TOPO® (Invitrogen, Carlsbad, USA) according to the manufacturer's instructions. The resulting plasmid pEXP5-NT/TOPO®-OsHPPD is isolated from E. coli TOP10 by performing a plasmid minipreparation. The expression cassette encoding N-terminally His6-tagged OsHPPD is confirmed by DNA sequencing.

### Example 2

### Heterologous Expression and Purification of Recombinant HPPD Enzymes

Recombinant HPPD enzymes are produced and overexpressed in E. coli. Chemically competent BL21 (DE3) cells (Invitrogen, Carlsbad, USA) are transformed with pEXP5-NT/TOPO® (see EXAMPLE 1) according to the manufacturer's instructions.

Transformed cells are grown at 37° C. in LB broth (Invitrogen, Carlsbad, USA) supplemented with 100 μg/ml ampicillin. Proteins are expressed without induction by IPTG (Isopropyl-D-1-thiogalactopyranoside).

At an OD600 (optical density at 600 nm) of 4 to 5, cells are harvested by centrifugation (8000×g). The cell pellet is resuspended in binding buffer (50 mM sodium phosphate buffer, 0.5 M NaCl, 10 mM Imidazole, pH 7.0) supplemented with complete EDTA free protease mix (Roche-Diagnostics) and homogenized using an Avestin Press. The homogenate is cleared by centrifugation (20,000×g). Hiss-tagged HPPD or mutant variants are purified by affinity chromatography on a HisTrap™ HP Column (GE Healthcare, Munich, Germany) according to the manufacturer's instructions. Purified HPPD or mutant variants are dialyzed against 100 mM sodium phosphate buffer pH 7.0, supplemented with 10% glycerin and stored at −86° C. Protein content is determined according to Bradford using the Bio-Rad protein assay (Bio-Rad Laboratories, Hercules, USA). The purity of the enzyme preparation is estimated by SDS-PAGE.

### Example 3

### Assay for HPPD Activity

HPPD produces homogentisic acid and CO₂ from 4-hydroxyphenylpyruvate (4-HPP) and O₂. The activity assay for HPPD is based on the analysis of homogentisic acid by reversed phase HPLC.

### Method (A)

The assay mixture can contain 150 mM potassium phosphate buffer pH 7.0, 50 mM L-ascorbic acid, 1 μM FeSO₄ and 7 μg of purified enzyme in a total volume of 1 ml.

Inhibitors are dissolved in DMSO (dimethylsulfoxide) to a concentration of 20 mM or 0.5 mM, respectively. From this stock solution serial five-fold dilutions are prepared in DMSO, which are used in the assay. The respective inhibitor solution accounts for 1% of the assay volume. Thus, final inhibitor concentrations range from 200 μM to 2.5 nM or from 5 μM to 63 pM, respectively.

After a preincubation of 30 min the reaction is started by adding 4-HPP to a final concentration of 0.1 mM. The reaction is allowed to proceed for 120 min at room temperature. The reaction is stopped by addition of 100 μl of 4.5 M phosphoric acid.

The sample is extracted on an Oasis® HLB cartridge 3 cc/60 mg (Waters) that was preequilibrated with 63 mM phosphoric acid. L-ascorbic acid is washed out with 3 ml of 63 mM phosphoric acid. Homogentisate is eluted with 1 ml of a 1:1 mixture of 63 mM phosphoric acid and methanol (w/w).

10 μl of the eluate is analyzed by reversed phase HPLC on a Symmetry® C18 column (particle size 3.5 μm, dimensions 4.6×100 mm; Waters) using 5 mM H₃PO₄/15% ethanol (w/w) as an eluent.

Homogentisic acid is detected electrochemically and quantified by measuring peak areas (Empower software; Waters).

Activities are normalized by setting the uninhibited enzyme activity to 100%. IC₅₀ values are calculated using non-linear regression.

### Method (B)

The assay mixture can contain 150 mM potassium phosphate buffer pH 7.0, 50 mM L-ascorbic acid, 100 μM Catalase (Sigma-Aldrich), 1 μM FeSO₄ and 0.2 units of purified HPPD enzyme in a total volume of 505 μl. 1 unit is defined as the amount of enzyme that is required to produce 1 nmol of HGA per minute at 20° C.

After a preincubation of 30 min the reaction is started by adding 4-HPP to a final concentration of 0.05 mM. The reaction is allowed to proceed for 45 min at room temperature. The reaction is stopped by the addition of 50 μl of 4.5 M phosphoric acid. The sample is filtered using a 0.2 μM pore size PVDF filtration device.

5 μl of the cleared sample is analyzed on an Atlantis T3 column (particle size 3 μm, dimensions 3×50 mm; Waters) by isocratic elution using 90% 10 mM NaH2PO₄ pH 2.2, 10% methanol (v/v).

HGA is detected electrochemically at 750 mV (mode: DC; polarity: positive) and quantified by integrating peak areas (Empower software; Waters).

Inhibitors are dissolved in DMSO (dimethylsulfoxide) to a concentration of 0.5 mM. From this stock solution serial five-fold dilutions are prepared in DMSO, which are used in the assay. The respective inhibitor solution accounts for 1% of the assay volume. Thus, final inhibitor concentrations range from 5 μM to 320 pM, respectively. Activities are normalized by setting the uninhibited enzyme activity to 100%. IC₅₀ values are calculated using non-linear regression.

### Example 4

### In Vitro Characterization of Wildtype HPPD Enzymes

Using methods which are described in the above examples or well known in the art, purified, recombinant wildtype HPPD enzymes are characterized with respect to their kinetic properties and sensitivity towards HPPD inhibiting herbicides. Apparent michaelis constants (Kₘ) and maximal reaction velocities (Vₘₐₓ) are calculated by non-linear regression with the software GraphPad Prism 5 (GraphPad Software, La Jolla, USA) using a substrate inhibition model. Apparent k_{cat} values are calculated from Vₘₐₓ assuming 100% purity of the enzyme preparation. Weighted means (by standard error) of Kₘ and IC₅₀ values are calculated from at least three independent experiments. The Cheng-Prusoff equation for competitive inhibition (Cheng, Y. C.; Prusoff, W. H. Biochem Pharmacol 1973, 22, 3099-3108) is used to calculate dissociation constants (Kᵢ). Examples of the data obtained are depicted in Table 11.

TABLE 11
Determination of michaelis constants (Kₘ) for 4-HPP,
turnover numbers (k_{cat}), catalytic efficiencies (k_{cat}/Kₘ) and
dissociation constants (Kᵢ) for various HPPD enzymes

| | | | | | | Kᵢ |
|---|---|---|---|---|---|---|
| | Kₘ | | k_{cat}/Kₘ | Kᵢ [nM] | Kᵢ [nM] | [nM] |
| | [μM] | k_{cat} | [μM⁻¹ | (inhibitor | (inhibitor | (Topra- |
| Enzyme | (4-HPP) | [s⁻¹]* | s⁻¹] | 1)** | 2)** | mezone) |
| Arabidopsis | 13 | 12.91 | 1.00 | 3 | 13 | 4 |
| HPPD | | (2.84) | | | | |
| Chlamydo- | 54 | 4.12 | 0.08 | 29 | 139 | 38 |
| monas HPPD1 | | (0.64) | | | | |
| Chlamydo- | 26 | 9.84 | 0.38 | 8 | n.d. | n.d. |
| monas HPPD2 | | (0.71) | | | | |

*Standard errors in parentheses
**“coumarone-derivative herbicides” used in this example are 3-[2,4-dichloro-3-(3-methyl-4,5-dihydroisoxazol-5-yl)phenyl]-1-(2,2-difluoroethyl)-2,2-dioxo-pyrido[3,2-c]thiazin-4-ol (Inhibitor 1) and 3-(2,4-dichlorophenyl)-1-(2,2-difluoroethyl)-2,2-dioxo-pyrido[3,2-c]thiazin-4-ol (Inhibitor 2) [see Formula No. 13 of Table 2]

It can be seen from the above examples that an HPPD enzyme can be selected as one which is resistant to “coumarone-derivative herbicides” because it is found that the dissociation constants governing dissociation of “coumarone-derivative herbicides” from complexes with this HPPD enzyme are greater than those governing dissociation of “coumarone-derivative herbicides” from complexes with other HPPD enzymes.

The above examples also indicate that selected HPPD enzymes, like Chlamydomonas HPPD1, are especially useful in the context of the current invention because their dissociation constants towards “coumarone-derivative herbicides” are greater than those from other HPPD enzymes, like the Arabidopsis HPPD.

It is evident that any HPPD enzyme that is resistant to “coumarone-derivative herbicides”, even if this protein is not exemplified in this text, is part of the subject-matter of this invention. Furthermore, the examples indicate that an HPPD enzyme can be selected as one which is resistant to Topramezone because it is found that the dissociation constant governing dissociation of Topramezone from complexes with this HPPD enzyme is greater than those governing dissociation of Topramezone from complexes with other HPPD enzymes.

### Example 5

### Rational Mutagenesis

By means of structural biology and sequence alignment it is possible to choose a certain number of amino acids which are found to be involved in the binding of “coumarone-derivative herbicides” and then to mutagenize them and obtain tolerant HPPD enzymes.

### (A) Site-Directed Mutagenesis

PCR-based site directed mutagenesis of pEXP5-NT/TOPO®-AtHPPD is done with the QuikChange II Site-Directed Mutagenesis Kit (Stratagene, Santa Clara, USA) according to the manufacturers instructions. This technique requires two chemically synthesized DNA primers (forward and reverse primer) for each mutation. Primers used for site directed mutagenesis of AtHPPD are listed in Table 12.

TABLE 12
PCR primers for site directed mutagenesis of AtHPPD
(SEQ ID NOs: 32 to 67)

| Primer | | Mutation |
|---|---|---|
| name | Primer sequence (5′ → 3′) | AtHPPD |
| HuJ141 | GAGGATTCGACTTCGCGCCTTCTCCTCC | Met335 → Ala |
| HuJ142 | GGAGGAGAAGGCGCGAAGTCGAATCCTC | Met335 → Ala |
| HuJ143 | GAGGATTCGACTTCTGGCCTTCTCCTCCG | Met335 → Trp |
| HuJ144 | CGGAGGAGAAGGCCAGAAGTCGAATCCTC | Met335 → Trp |
| HuJ145 | GGAGGATTCGACTTCTTTCCTTCTCCTCCGC | Met335 → Phe |
| HuJ146 | GCGGAGGAGAAGGAAAGAAGTCGAATCCTCC | Met335 → Phe |
| HuJ147 | GTGACAGGCCGACGATAGCTATAGAGATAATCCAG | Phe392 → Ala |
| HuJ148 | CTGGATTATCTCTATAGCTATCGTCGGCCTGTCAC | Phe392 → Ala |
| HuJ153 | GACTTCATGCCTCCTCCTCCGCCTACTTAC | Ser337 → Pro |
| HuJ154 | GTAAGTAGGCGGAGGAGGAGGCATGAAGTC | Ser337 → Pro |
| HuJ155 | GATTCGACTTCATGGCTTCTCCTCCGCCTAC | Pro336 → Ala |
| HuJ156 | GTAGGCGGAGGAGAAGCCATGAAGTCGAATC | Pro336 → Ala |
| HuJ157 | CAGATCAAGGAGTGTCAGGAATTAGGGATTCTTG | Glu363 → Gln |
| HuJ158 | CAAGAATCCCTAATTCCTGACACTCCTTGATCTG | Glu363 → Gln |
| HuJ159 | CGGAACAAAGAGGAAGAGTGAGATTCAGACGTATTTGG | Gln293 → Val |
| HuJ160 | CCAAATACGTCTGAATCTCACTCTTCCTCTTTGTTCCG | Gln293 → Val |
| HuJ169 | CGTTGCTTCAAATCTTCCCGAAACCACTAGGTGACAGGCC | Thr382 → Pro |
| HuJ170 | GGCCTGTCACCTAGTGGTTTCGGGAAGATTTGAAGCAACG | Thr382 → Pro |
| HuJ171 | CAAATCTTCACAAAACCAGTGGGTGACAGGCCGACGAT | Leu385 → Val |
| HuJ172 | ATCGTCGGCCTGTCACCCACTGGTTTTGTGAAGATTTG | Leu385 → Val |
| HuJ173 | TGACAGGCCGACGATATTTCTGGAGATAATCCAGAGAGTA | Ile393 → Leu |
| HuJ174 | TACTCTCTGGATTATCTCCAGAAATATCGTCGGCCTGTCA | Ile393 → Leu |
| HuJ175 | GACTTCATGCCTGCGCCTCCGCCTACTTAC | Ser337 → Ala |
| HuJ176 | GTAAGTAGGCGGAGGCGCAGGCATGAAGTC | Ser337 → Ala |
| HuJ177 | GGCAATTTCTCTGAGTTCTTCAAGTCCATTGAAG | Leu427 → Phe |
| HuJ178 | CTTCAATGGACTTGAAGAACTCAGAGAAATTGCC | Leu427 → Phe |
| HuJ185 | GGAACAAAGAGGAAGAGTGTGATTCAGACGTATTTGG | Gln293 → Val |
| HuJ186 | CCAAATACGTCTGAATCACACTCTTCCTCTTTGTTCC | Gln293 → Val |
| Ta2-55 | GAGGATTCGACTTCAACCCTTCTCCTCC | Met335 → Asn |
| Ta2-56 | GGAGGAGAAGGGTTGAAGTCGAATCCTC | Met335 → Asn |
| Ta2-57 | GAGGATTCGACTTCCAGCCTTCTCCTCC | Met335 → Gln |
| Ta2-58 | GGAGGAGAAGGCTGGAAGTCGAATCCTC | Met335 → Gln |
| Ta2-59 | GGAACAAAGAGGAAGAGTAACATTCAGACGTATTTGG | Gln293 → Asn |
| Ta2-60 | CCAAATACGTCTGAATGTTACTCTTCCTCTTTGTTCC | Gln293 → Asn |
| Ta2-61 | GGAACAAAGAGGAAGAGTCACATTCAGACGTATTTGG | Gln293 → His |
| Ta2-62 | CCAAATACGTCTGAATGTGACTCTTCCTCTTTGTTCC | Gln293 → His |

Mutant plasmids are isolated from E. coli TOP10 by performing a plasmid minipreparation and confirmed by DNA sequencing.

The combination of single amino acid substitutions is achieved by a stepwise mutagenesis approach.

### (B) In Vitro Characterization of Arabidopsis HPPD Mutants

Purified, mutant HPPD enzymes are obtained by the methods described above. Dose response and kinetic measurements are carried out using the described HPPD activity assay. Apparent michaelis constants (Kₘ) and maximal reaction velocities (Vₘₐₓ) are calculated by non-linear regression with the software GraphPad Prism 5 (GraphPad Software, La Jolla, USA) using a substrate inhibition model. Apparent k_{cat} values are calculated from Vₘₐₓ assuming 100% purity of the enzyme preparation. Weighted means (by standard error) of Kₘ and IC₅₀ values are calculated from at least three independent experiments. The Cheng-Prusoff equation for competitive inhibition (Cheng, Y. C.; Prusoff, W. H. Biochem Pharmacol 1973, 22, 3099-3108) is used to calculate dissociation constants (Kᵢ). Examples of the data obtained are depicted in Table 13.

TABLE 13
Determination of michaelis constants (Kₘ) for 4-HPP, turnover
numbers (k_{cat}), catalytic efficiencies (k_{cat}/Kₘ) and dissociation
constants (Kᵢ) for variants of the Arabidopsis HPPD enzyme

| Arabidopsis | | | k_{cat}/Kₘ | Kᵢ [nM] | Kᵢ [nM] | Kᵢ [nM] |
|---|---|---|---|---|---|---|
| HPPD | Kₘ [μM] | k_{cat} | [μM⁻¹ | (inhibitor | (inhibitor | (Topra- |
| variant | (4-HPP) | [s⁻¹]* | s⁻¹] | 1)** | 2)** | mezone) |
| Wild-type | 13 | 12.91 | 1.00 | 3 | 13 | 4 |
| | | (2.84) | | | | |
| Q293H | 104 | 3.34 | 0.03 | 23 | 19 | 14 |
| | | (1.15) | | | | |
| Q293N | 56 | 0.81 | 0.01 | 41 | 44 | 36 |
| | | (0.20) | | | | |
| M335N | 112 | 7.62 | 0.07 | 20 | n.d. | n.d. |
| | | (1.00) | | | | |
| M335Q | 129 | 6.54 | 0.05 | 24 | n.d. | n.d. |
| | | (0.70) | | | | |
| P336A | 37 | 12.27 | 0.33 | 13 | n.d. | n.d. |
| E363Q | | (0.84) | | | | |
| L385V | 36 | 7.07 | 0.20 | 19 | n.d. | n.d. |
| | | (0.86) | | | | |
| I393L | 46 | 9.23 | 0.20 | 21 | n.d. | n.d. |
| | | (0.72) | | | | |

*Standard errors in parentheses
**“coumarone-derivative herbicides” used in this study are 3-[2,4-dichloro-3-(3-methyl-4,5-dihydroisoxazol-5-yl)phenyl]-1-(2,2-difluoroethyl)-2,2-dioxo-pyrido[3,2-c]thiazin-4-ol (Inhibitor 1) and 3-(2,4-dichlorophenyl)-1-(2,2-difluoroethyl)-2,2-dioxo-pyrido[3,2-c]thiazin-4-ol (Inhibitor 2)

It can be seen from the above examples that a mutant HPPD enzyme can be selected as one which is resistant to “coumarone-derivative herbicides” because it is found that the dissociation constants governing dissociation of “coumarone-derivative herbicides” from complexes with HPPD mutants are greater than those governing dissociation of “coumarone-derivative herbicides” from complexes with the wildtype HPPD enzyme. The above examples also indicate that selected HPPD mutants, like I393L, L385V, or P336A E363Q, are especially useful in the context of the current invention because their catalytic efficiencies (k_{cat}/Kₘ) are decreased by a maximum of only five fold, as compared to the wildtype enzyme.

Furthermore, the examples indicate that a mutant HPPD enzyme can be selected as one which is resistant to Topramezone because it is found that the dissociation constants governing dissociation of Topramezone from complexes with HPPD mutants are greater than those governing dissociation of Topramezone from complexes with the wildtype HPPD enzyme.

### Example 6

### Random Mutagenesis and Screening of Algae Cells to Identify Clones which are Tolerant to “Coumarone-Derivative Herbicides” and Identification of Causative Mutations in HPPD/HST Genes

Bleaching herbicides with a mode of action in plastoquinone or tocopherol biosynthesis can inhibit algae growth (Tables 14 and 15). These effects can be partly reversed by intermediates of homogentisic acid biosynthesis (Table 14). To generate mutations conferring “coumarone-derivative herbicide” resistance in HPPD or HST genes, chemical or UV mutagenesis can be used. Especially unicellular organisms like Chlamydomonas reinhardtii or Scenedesmus obliquus are useful for identifying dominant mutations in herbicide resistance.

TABLE 14
C. reinhardtii growth inhibition by HPPD inhibiting herbicides and
the effect of homogentisic acid

| | | Growth | |
|---|---|---|---|
| | | inihibition [%] | |
| | | C. reinhardtii | |
| | | (CC-503) | |
| Compound [No 1, 2 of | | | + Homogentisic |
| Table 2] | c [M] | | acid |
| 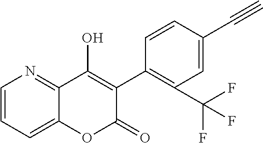 [SMILES: C#Cc1ccc(-c2c(O)c3ncccc3oc2=O)c(C(F)(F)F)c1] | 1 * 10⁻⁴ | 61 | 43 |
| b]pyridin-2-one) | 5 * 10⁻⁴ | 90 | 67 |
| Topramezone | 1 * 10⁻⁴ | 100 | 80 |
| | 5 * 10⁻⁴ | 100 | 100 |

TABLE 15
S. obliquus growth inhibition by a “coumarone-derivative herbicide”

| | | Growth |
|---|---|---|
| | | inihibition [%] |
| Compound [No 1, 2 of | | Scenedesmus |
| Table 2] | c [M] | obliquus |
| 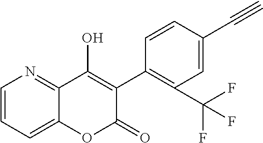 [SMILES: C#Cc1ccc(-c2c(O)c3ncccc3oc2=O)c(C(F)(F)F)c1] (3-[4-ethynyl-2- (trifluoromethyl)phenyl]-4- hydroxy-pyrano[3,2- b]pyridin-2-one) | 1 * 10⁻⁵ 1 * 10⁻⁴ | 77 100 |

Algae cells of Chlamydomonas reinhardtii strains CC-503 and CC-1691 (Duke University, Durham, USA) are propagated in TAP medium (Gorman and Levine (1965) PNAS 54: 1665-1669) by constant shaking at 100 rpm, 22° C. and 30 μmol Phot*m⁻²*s⁻² light illumination. Scenedesmus obliquus (University of Gottingen, Germany) are propagated in algae medium as described (Boger and Sandmann, (1993) In: Target assays for modern herbicides and related phytotoxic compounds, Lewis Publishers) under same culturing conditions as mentioned for Chlamydomonas. Compound screening is performed at 450 μmol Phot*m⁻²*s⁻² illumination.

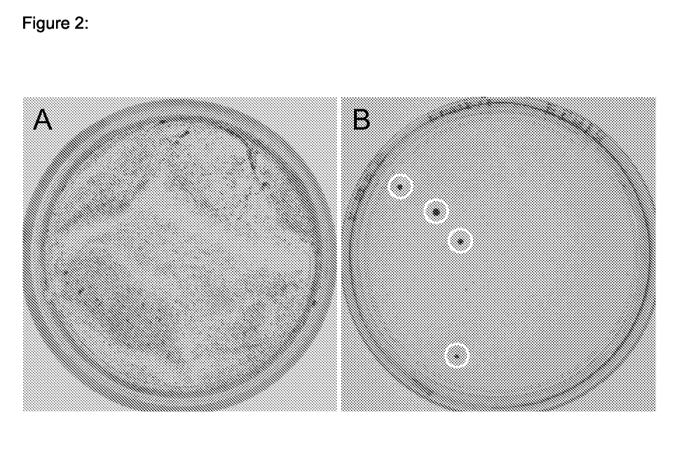

Sensitive strains of Chlamydomonas reinhardtii or Scenedesmus obliquus (Tables 14, 15) are mutated with 0.14 Methylmethanesulfonate (EMS) for 1 h as described by Loppes (1969, Mol Gen Genet. 104: 172-177) Tolerant strains are identified by screening of mutagenized cells on solid nutrient solution plates containing “coumarone-derivative herbicides” or other HPPD inhibiting herbicides at wildype-lethal concentrations. Examples of the data obtained are depicted in Table 16 and FIG. 2.

TABLE 16
Tolerance of identified Chlamydomonas strains to “coumarone-
derivative herbicides”, Topramezone and Mesotrione. IC₅₀ values
[mol/l] of growth inhibition are depicted.

| | Strain | | | | | | |
|---|---|---|---|---|---|---|---|
| | CC196 | | | | | | |
| | 1 wild- | | | | | | |
| Herbicide | type | CMr04 | CMr05 | CMr06 | CMr10 | CMr13 | CMr15 |
| “coumarone- | 7.6 * 10⁻⁴ | >1.0 * 10⁻³ | >1.0 * 10⁻³ | >1.0 * 10⁻³ | >1.0 * 10⁻³ | 9.5 * 10⁻⁴ | >1.0 * 10⁻³ |
| derivative | | | | | | | |
| herbicides” 1 | | | | | | | |
| (4-hydroxy-3-[2- | | | | | | | |
| methyl-3-(5-methyl- | | | | | | | |
| 4,5-dihydroisoxazol- | | | | | | | |
| 3-yl)-4- | | | | | | | |
| methylsulfonyl- | | | | | | | |
| phenyl]pyrano[3,2- | | | | | | | |
| b]pyridin-2-one) | | | | | | | |
| [see No: 8 of Table | | | | | | | |
| 2] | | | | | | | |
| “coumarone- | 6.2 * 10⁻⁴ | >1.0 * 10⁻³ | >1.0 * 10⁻³ | >1.0 * 10⁻³ | >1.0 * 10⁻³ | 8.1 * 10⁻⁴ | >1.0 * 10⁻³ |
| derivative | | | | | | | |
| herbicides” 2 | | | | | | | |
| (3-[2,4-dichloro-3- | | | | | | | |
| (3-methyl-4,5- | | | | | | | |
| dihydroisoxazol-5- | | | | | | | |
| yl)phenyl]-1-(2,2- | | | | | | | |
| difluoroethyl)-2,2- | | | | | | | |
| dioxo-pyrido[3,2- | | | | | | | |
| c]thiazin-4-ol) | | | | | | | |
| [see No: 13 of Table | | | | | | | |
| 2] | | | | | | | |
| Mesotrione | 3.0 * 10⁻⁵ | >6.0 * 10⁻⁴ | >6.0 * 10⁻⁴ | >6.0 * 10⁻⁴ | 4.5 * 10⁻⁴ | >6.0 * 10⁻⁴ | 5.4 * 10⁻⁴ |
| Topramezone | 1.4 * 10⁻⁴ | 3.9 * 10⁻⁴ | 7.1 * 10⁻⁴ | 8.6 * 10⁻⁴ | 9.2 * 10⁻⁴ | 2.0 * 10⁻⁴ | 2.3 * 10⁻⁴ |

It can be seen from the above examples that a mutagenized Chlamydomonas strain can be selected as one which is resistant to “coumarone-derivative herbicides” because it is found that a mutagenized strain which was selected on “coumarone-derivative herbicide” containing medium shows higher IC50 values and thus less growth inhibition than a wild type strain. Furthermore, the examples indicate that a mutagenized Chlamydomonas strain can be selected as one which is resistant to other HPPD-inhibiting herbicides, like Mesotrione or Topramezone, because it is found that a mutagenized strain which was selected on medium containing these herbicides shows higher IC50 values and thus less growth inhibition than a wild type strain.

The above examples also indicate that selected mutants show a high level of tolerance or a broad cross resistance against all of the tested compounds (e.g. CMr06)

Amplification of HPPD and HST genes from wild-type and resistant Chlamydomonas reinhardtii from genomic DNA or copy DNA as template are performed by standard PCR techniques with DNA oligonucleotides as listed in Table 17. DNA oligonucleotides are derived from SEQ ID NO: 3, 5 and 7. The resulting DNA molecules are cloned in standard sequencing vectors and sequenced by standard sequencing techniques. Mutations are identified by comparing wildtype and mutant HPPD/HST sequences by the sequence alignment tool Align X (Vector NTI Advance Software Version 10.3, Invitrogen, Carlsbad, USA).

TABLE 17
PCR primers for amplification of CrHPPD1,
CrHPPD2 and CrHST (SEQ ID NOs: 68 to 73)

| | Primer sequence |
|---|---|
| Primer name | (5′-3′) |
| Cr_HPPD1_Fw | ATGGGCGCTGGTGGCGCTTCTAC |
| Cr_HPPD1_Rv | CTACACATTTAGGGTGCGCTCATAGTCC |
| Cr_HPPD2_Fw | ATGGGAGCGGGTGGTGCAGGCAC |
| Cr_HPPD2_Rv | TTAAACATTTAAGGTGCGCTCATAGTCCTC |
| Cr_HST_Fw | ATGGACCTTTGCAGCTCAACTGGAAG |
| Cr_HST_Rv | GTACGCGCTGCTGCCGTTCCTGTAG |

An example of the data obtained is depicted in Table 18.

TABLE 18
CrHPPD2 mutation identified in the “coumarone-derivative”
herbicide tolerant Chlamydomonas strain CMr15

| Strain | Mutation (nucleotide exchange) | Amino acid exchange |
|---|---|---|
| CMr15 | G1252A (in SEQ ID No: 5) | A418T (in SEQ ID NO: 6) |

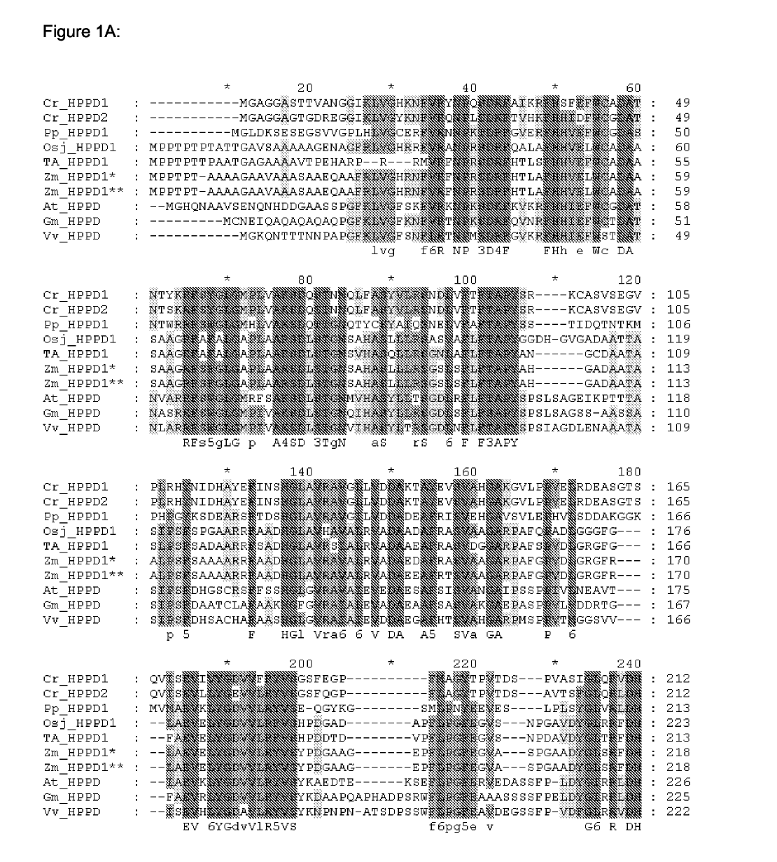

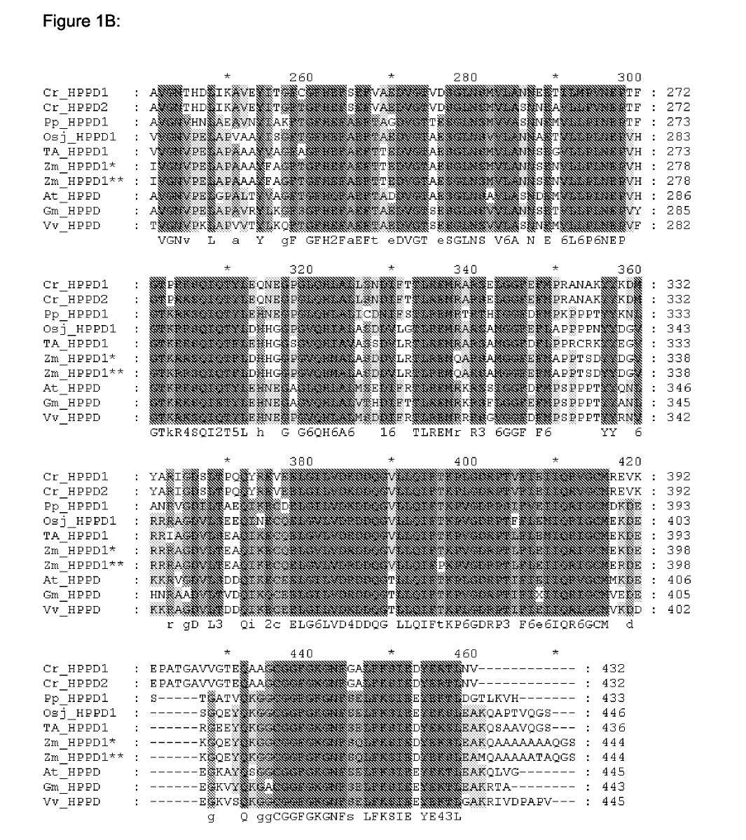

To identify orthologe HPPD and HST genes from Scenedesmus obliquus, degenerated PCR primer are defined from conserved regions based on protein alignments of HPPD or HST respectively (FIGS. 1A and B). Forward primers for HPPD are generated from consensus sequence R-K-S-Q-I-Q-T (Table 19A) or S-G-L-N-S-A/M/V-V-L-A (Table 19B), reverse primers are derived from consensus sequence Q-(I/V)-F-T-K-P-(L/V) (Table 19A) or C-G-G-F-GK-G-N-F (Table 19B). Forward primers for HST are generated from consensus sequence WK-F-L-R-P-H-T-I-R-G-T, reverse primers are derived from consensus sequence F-Y-R-F/W-I-W-N-L-F-Y-A/S/V (Table 19). Based on the received HPPD/HST gene sequence tags, protein coding sequences are completed by adapter PCR or TAIL PCR techniques as described by Liu and Whittier (1995, Genomics 25: 674-681) and Yuanxin et al. (2003 Nuc Acids R^{e}— search 31: 1-7) or Spertini et al. (1999 Biotechniques 27: 308-314) on copy DNA or genomic DNA.

TABLE 19A
PCR primers for partial amplification
of SoHPPD (SEQ ID NOs: 74 to 77)

| | Primer sequence |
|---|---|
| Primer name | (5′-3′) |
| So_Deg_HPPD_Fw | MGBAARWSYCAGATYCAGAC |
| So_Deg_HPPD_Rv | ASIGGYTTIGTRAAVAYCTG |
| So_Deg_HST_Fw | TGGMGNTTYYTNMGNCCNCAYACNATHMG |
| So_Deg_HST_Rv | YTCNGCNNHRAANARRTTCCADATVMANC |

Wherein “I” in So_Deg_HPPD_Rv stands for inositol but can also be any nucleotide a, g, t, c

TABLE 19B
PCR primers for partial amplification
of SoHPPD (SEQ ID NOs: 78 to 81)

| | Primer sequence |
|---|---|
| Primer name | (5′-3′) |
| So_Deg_HPPD_Fw2 | WSNGGNYTNAAYWSNRYNGTNYTNGC |
| So_Deg_HPPD_Rv2 | RAARTTNCCYTTNCCRAANCCNCCRC |
| So_Deg_HST_Fw2 | TGGMGNTTYYTNMGNCCNCAYACNATHMG |
| So_Deg_HST_Rv2 | YTCNGCNNHRAANARRTTCCADATVMANC |

### Example 7

### Screening of EMS Mutagenized Arabidopsis Thaliana Population to Identify Herbicide Tolerant Plants and Identification of Causative Mutations in HPPD/HST Genes

A M2 population of EMS treated Arabidopsis thaliana plants are obtained from Lehle Seeds (Round Rock, Tex., USA). Screenings are done by plating Arabidopsis seeds on half-strength murashige skoog nutrient solution containing 0.5% gelating agent Gelrite® and coumarone-derivative herbicide of 0.1 to 100 μM, depending on compound activity. Plates are incubated in a growth chamber in 16:8 h light:dark cycles at 22° C. for up to three weeks. Tolerant plants showing less intense bleaching phenotypes are planted in soil and grown to maturity under greenhouse conditions. In rosette plant stage, leaf discs are harvested from coumarone-derivative herbicide tolerant plants for isolation of genomic DNA with DNeasy Plant Mini Kit (Qiagen, Hilden, Germany) or total mRNA with RNeasy Plant Mini Kit (Quagen, Hilden, Germany). HPPD or HST sequences are amplified by standard PCR techniques from genomic DNA with the respective oligonucleotides as described in Table 11. For amplification of HPPD or HST from mRNA, copy DNA are synthesized with Superscript III Reverse Transcriptase (Invitrogene, Carlsbad, Calif., USA) and HPPD or HST are amplified with DNA oligonucleotides listed in Table 11. After cloning of PCR products in standard sequencing plasmid, DNA sequence of mutated HPPD/HST genes are identified by standard sequencing techniques. Mutations are identified by comparing wildtype and mutant HPPD/HST sequences by sequence alignment tool Align X (Vector NTI Advance Software Version 10.3, Invitrogene, Carlsbad, Calif., USA).

TABLE 20
PCR primers for amplification of
AtHPPD and AtHST
(SEQ ID NOs: 82 to 85)

| Primer | Primer sequence |
|---|---|
| name | (5′-3′) |
| At_HPPD_Fw | ATGGGCCACCAAAACGCCGC |
| At_HPPD_Rv | TCATCCCACTAACTGTTTGGCTTCAAG |
| At_HST_Fw | ATGGAGCTCTCGATCTCACAATC |
| At_HST_Rv | CTAGAGGAAGGGGAATAACAGATACTC |

### Example 8

### Preparation of Plants which Express Heterologous HPPD and/or HST Enzymes and which are Tolerant to “Coumarone-Derivative Herbicides”

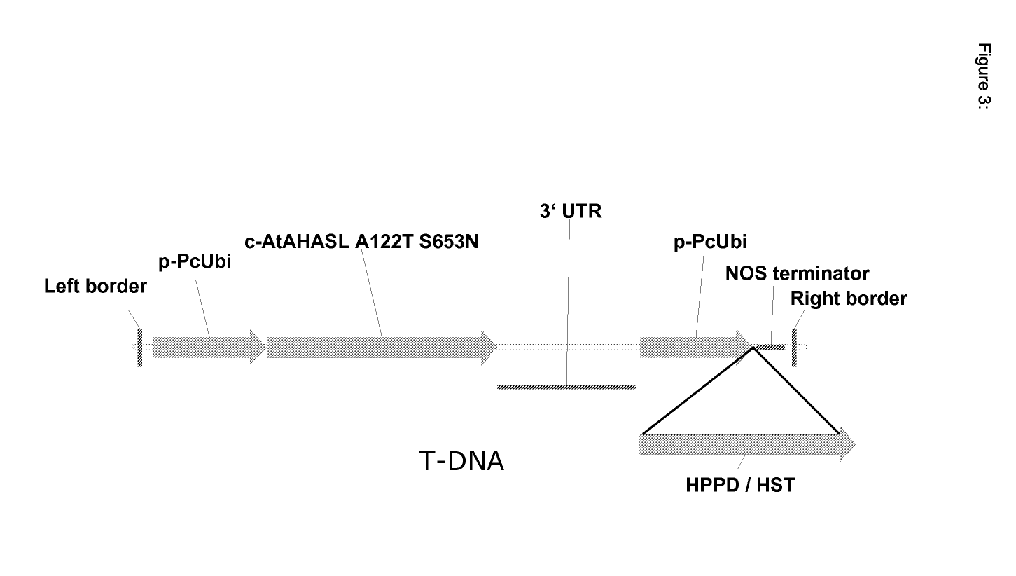

Various methods for the production of stably transformed plants are well known in the art. coumarone-derivative herbicidetolerant soybean (Glycine max) plants can be produced by a method described by Olhoft et al. (US patent 2009/0049567). Briefly, HPPD or HST encoding polynucleotides are cloned into a binary vector using standard cloning techniques as described by Sambrook et al. (Molecular cloning (2001) Cold Spring Harbor Laboratory Press). The final vector construct contains an HPPD or HST encoding sequence flanked by a promoter sequence (e.g. the ubiquitin promoter (PcUbi) sequence) and a terminator sequence (e.g. the nopaline synthase terminator (NOS) sequence) and a resistance marker gene cassette (e.g. AHAS) (FIG. 3). Optionally, the HPPD or HST gene can provide the means of selection. Agrobacterium-mediated transformation is used to introduce the DNA into soybean's axillary meristem cells at the primary node of seedling explants. After inoculation and co-cultivation with Agrobacteria, the explants are transferred to shoot induction medium without selection for one week. The explants are subsequently transferred to shoot induction medium with 1-3 μM imazapyr (Arsenal) for 3 weeks to select for transformed cells. Explants with healthy callus/shoot pads at the primary node are then transferred to shoot elongation medium containing 1-3 μM imazapyr until a shoot elongates or the explant dies. After regeneration, transformants are transplanted to soil in small pots, placed in growth chambers (16 hr day/8 hr night; 25° C. day/23° C. night; 65% relative humidity; 130-150 mE m-2 s-1) and subsequently tested for the presence of the T-DNA via Taqman analysis. After a few weeks, healthy, transgenic positive, single copy events are transplanted to larger pots and allowed to grow in the growth chamber.

Transformation of corn plants is done by a method described by McElver and Singh (WO 2008/124495). Plant transformation vector constructs containing HPPD or HST sequences are introduced into maize immature embryos via Agrobacterium-mediated transformation. Transformed cells are selected in selection media supplemented with 0.5-1.5 μM imazethapyr for 3-4 weeks. Transgenic plantlets are regenerated on plant regeneration media and rooted afterwards. Transgenic plantlets are subjected to TaqMan analysis for the presence of the transgene before being transplanted to potting mixture and grown to maturity in greenhouse. Arabidopsis thaliana is transformed with HPPD or HST sequences by floral dip method as described by McElver and Singh (WO 2008/124495).

Transformation of Oryza sativa (rice) are done by protoplast transformation as described by Peng et al. (U.S. Pat. No. 6,653,529)

T0 or T1 transgenic plant of soybean, corn, rice and Arabidopsis thaliana containing HPPD or HST sequences are tested for improved tolerance to “coumarone-derived herbicides” in greenhouse studies.

### Example 9

### Greenhouse Experiments

Transgenic plants expressing heterologous HPPD or HST enzymes are tested for tolerance against coumarone-derivative herbicides in greenhouse experiments.

For the pre-emergence treatment, the herbicides are applied directly after sowing by means of finely distributing nozzles. The containers are irrigated gently to promote germination and growth and subsequently covered with transparent plastic hoods until the plants have rooted. This cover causes uniform germination of the test plants, unless this has been impaired by the herbicides.

For post emergence treatment, the test plants are first grown to a height of 3 to 15 cm, depending on the plant habit, and only then treated with the herbicides. For this purpose, the test plants are either sown directly and grown in the same containers, or they are first grown separately and transplanted into the test containers a few days prior to treatment.

For testing of T0 plants, cuttings can be used. In the case of soybean plants, an optimal shoot for cutting is about 7.5 to 10 cm tall, with at least two nodes present. Each cutting is taken from the original transformant (mother plant) and dipped into rooting hormone powder (indole-3-butyric acid, IBA). The cutting is then placed in oasis wedges inside a bio-dome. Wild type cuttings are also taken simultaneously to serve as controls. The cuttings are kept in the bio-dome for 5-7 days and then transplanted to pots and then acclimated in the growth chamber for two more days. Subsequently, the cuttings are transferred to the greenhouse, acclimated for approximately 4 days, and then subjected to spray tests as indicated. Depending on the species, the plants are kept at 10-25° C. or 20-35° C. The test period extends over 3 weeks. During this time, the plants are tended and their response to the individual treatments is evaluated. Herbicide injury evaluations are taken at 2 and 3 weeks after treatment. Plant injury is rated on a scale of 0 to 9, 0 being no injury and 9 being complete death.

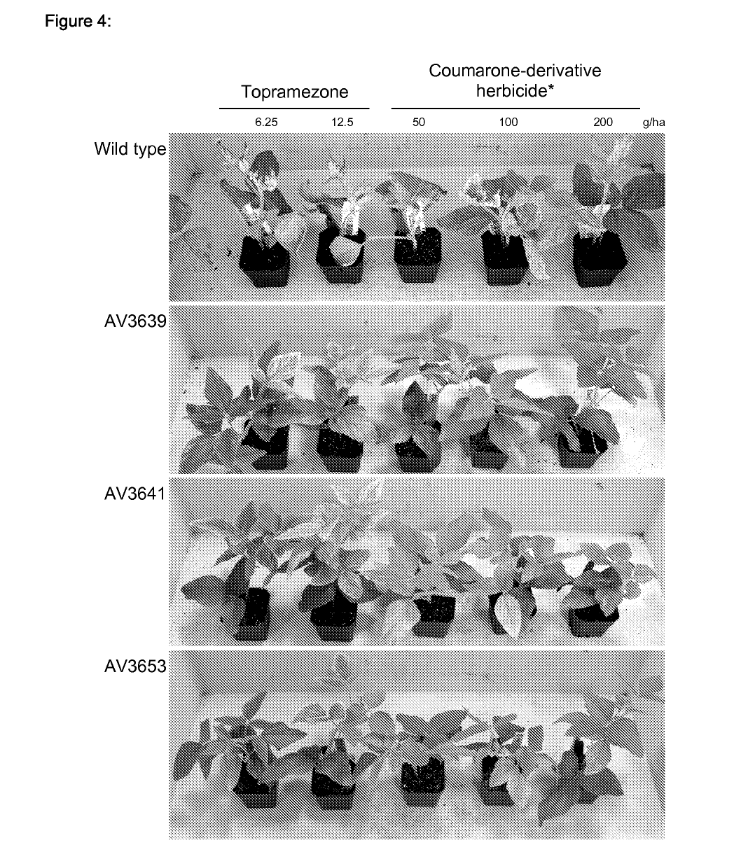

Examples of the data obtained are depicted in Table 21 and in FIG. 4.

TABLE 21
Greenhouse testing of transgenic soybean plants (T0 cuttings). Injury evaluations were taken
two weeks after herbicide treatment.

| | Transgene | | | | | CrHPP | | | |
|---|---|---|---|---|---|---|---|---|---|
| | Event | none | AtHPPD | | | D1 | CrHPPD2 | | |
| | Dose | Wild | AV36 | AV36 | AV36 | AV364 | AV36 | LG45 | LG46 |
| Herbicide | [g/ha] | type | 53 | 41 | 39 | 6 | 44 | 64 | 28 |
| “coumarone- | 50 | 4.5 | 3 | 2 | 3 | 3 | 4 | 3 | 4 |
| derivative | 100 | 5.5 | 3 | 2 | 2 | 4 | 4 | 3 | 3 |
| herbicide”* | 200 | 6 | 3 | 3 | 3 | 4 | 5 | 4 | 4 |
| Topra- | 6.25 | 7 | 2 | 4 | 4 | 6 | 6 | 3 | 5 |
| mezone | 12.5 | 7 | 3 | 4 | 5 | 7 | 5 | 4 | 6 |

*3-[2,4-dichloro-3-(3-methyl-4,5-dihydroisoxazol-5-yl)phenyl]-1-(2,2-difluoroethyl)-2,2-dioxo-pyrido[3,2-c]thiazin-4-ol

It can be seen from the above examples that an HPPD encoding polynucleotide which is transformed into plants can be selected as one which confers resistance to coumarone-derivative herbicides because it is found that plants which are transformed with such a polynucleotide are less injured by coumarone-derivative herbicides than the non-transformed control plants.

Furthermore, the examples indicate that an HPPD encoding polynucleotide which is trans-formed to plants can be selected as one which confers resistance to Topramezone because it is found that plants which are transformed with such a polynucleotide are less injured by Topramezone than the non-transformed control plants.

1. A method for controlling undesired vegetation at a plant cultivation site, the method comprising the steps of:
a) providing, at said site, a plant that comprises at least one nucleic acid comprising:
(i) a nucleotide sequence encoding a wild-type hydroxyphenyl pyruvate dioxygenase (HPPD) or a mutated hydroxyphenyl pyruvate dioxygenase (mut-HPPD) which is resistant or tolerant to a coumarone-derivative herbicide; and/or
(ii) a nucleotide sequence encoding a wild-type homogentisate solanesyl transferase (HST) or a mutated homogentisate solanesyl transferase (mut-HST) which is resistant or tolerant to a coumarone-derivative herbicide; and
b) applying to said site an effective amount of said herbicide.

2. The method according to claim 1, wherein the nucleotide sequence of (i) comprises the nucleic acid sequence of SEQ ID NO: 1, 3, or 5, or a variant or derivative thereof.

3. The method according to claim 1, wherein the nucleotide sequence of (ii) comprises the nucleic acid sequence of SEQ ID NO: 7 or 9, or a variant or derivative thereof.

4. The method according to claim 1, wherein the plant comprises at least one additional heterologous nucleic acid comprising (iii) a nucleotide sequence encoding an herbicide tolerance enzyme.

5. The method according to claim 1, wherein the coumarone-derivative herbicide is applied in conjunction with one or more other HPPD- and/or HST targeting herbicides.

6. A method for identifying a coumarone-derivative herbicide comprising utilizing a mutated hydroxyphenyl pyruvate dioxygenase (mut-HPPD) encoded by a nucleic acid which comprises the nucleotide sequence of SEQ ID NO: 1, 3, or 5, or a variant or derivative thereof, and/or a mutated homogentisate solanesyl transferase (mut-HST) encoded by a nucleic acid which comprises the nucleotide sequence of SEQ ID NO: 7 or 9, or a variant or derivative thereof.

7. The method according to claim 6, comprising the steps of:
a) generating a transgenic cell or plant comprising a nucleic acid encoding a mut-HPPD, wherein the mut-HPPD is expressed;
b) applying a coumarone-derivative to the transgenic cell or plant of a) and to a control cell or plant of the same variety;
c) determining the growth or the viability of the transgenic cell or plant and the control cell or plant after application of said coumarone-derivative, and
d) selecting a coumarone-derivative which confers reduced growth to the control cell or plant as compared to the growth of the transgenic cell or plant.

8. A method of identifying a nucleotide sequence encoding a mutated hydroxyphenyl pyruvate dioxygenas (mut-HPPD) which is resistant or tolerant to a coumarone-derivative herbicide, the method comprising:
a) generating a library of mut-HPPD-encoding nucleic acids;
b) screening a population of the resulting mut-HPPD-encoding nucleic acids by expressing each of said nucleic acids in a cell or plant and treating said cell or plant with a coumarone-derivative;
c) comparing the “coumarone-derivative”-tolerance levels provided by said population of mut-HPPD encoding nucleic acids with the “coumarone-derivative”-tolerance level provided by a control HPPD-encoding nucleic acid; and
d) selecting at least one mut-HPPD-encoding nucleic acid that provides a significantly increased level of tolerance to a “coumarone-derivative” as compared to that provided by the control HPPD-encoding nucleic acid.

9. The method according to claim 8, wherein the mut-HPPD-encoding nucleic acid selected in step d) provides at least 2-fold as much tolerance to a coumarone-derivative herbicide as that provided by the control HPPD-encoding nucleic acid.

10. The method according to claim 8, wherein the resistance or tolerance is determined by generating a transgenic plant comprising a nucleic acid sequence of the library generated in step a) and comparing said transgenic plant with a corresponding control plant.

11. An isolated nucleic acid encoding a mut-HPPD, wherein the nucleic acid is identified by the method as defined in claim 8.

12. The nucleic acid according to claim 11, wherein the mut-HPPD is a variant of the amino acid sequence of SEQ ID NO: 2 which comprises one or more of the following mutations:
a) the amino acid at position 293 is other than glutamine;
b) the amino acid at position 335 is other than methionine;
c) the amino acid at position 336 is other than proline;
d) the amino acid at position 337 is other than serine;
e) the amino acid position 363 is other than glutamic acid;
f) the amino acid at position 422 is other than glycine;
g) the amino acid at position 385 is other than leucine; and/or
h) the amino acid position 393 is other than an isoleucine.

13. A transgenic plant cell transformed by a wild-type or mutated hydroxyphenyl pyruvate dioxygenase (mut-HPPD) nucleic acid, wherein expression of the nucleic acid in the plant cell results in increased resistance or tolerance to a coumarone-derivative herbicide as compared to a corresponding wild type plant cell.

14. The transgenic plant cell of claim 13, wherein the wild-type or mut-HPPD nucleic acid comprises a polynucleotide sequence selected from the group consisting of:
a) the polynucleotide sequence of SEQ ID NO: 1, 3 or 5, or a variant or derivative thereof;
b) the polynucleotide sequence of SEQ ID NO: 7 or 9, or a variant or derivative thereof;
c) a polynucleotide sequence encoding the polypeptide of SEQ ID NO: 2, 4, 6, 8, or 10, or a variant or derivative thereof;
d) a polynucleotide sequence comprising at least 60 consecutive nucleotides of any of a) through c); and
e) a polynucleotide sequence complementary to the polynucleotide sequence of any of a) through d).

15. The transgenic plant cell of claim 14, wherein the variant of the polypeptide of SEQ ID NO: 2 in c) comprises one or more of the following mutations:
a) the amino acid at position 293 is other than glutamine;
b) the amino acid at position 335 is other than methionine;
c) the amino acid at position 336 is other than proline;
d) the amino acid at position 337 is other than serine;
e) the amino acid position 363 is other than glutamic acid;
f) the amino acid at position 422 is other than glycine;
g) the amino acid at position 385 is other than leucine; and/or
h) the amino acid position 393 is other than an isoleucine.

16. A transgenic plant comprising the transgenic plant cell of claim 13, wherein expression of the nucleic acid in the plant increases resistance to a coumarone-derivative herbicide in the plant as compared to a corresponding wild type plant.

17. A plant that expresses a mutagenized or recombinant mutated hydroxyphenyl pyruvate dioxygenase (mut-HPPD) comprising a variant of the amino acid sequence of SEQ ID NO: 2 which differs from an amino acid sequence of HPPD of a corresponding wild-type plant at one or more amino acid positions, wherein the variant comprises one or more of the following mutations:
a) the amino acid at position 293 is other than glutamine;
b) the amino acid at position 335 is other than methionine;
c) the amino acid at position 336 is other than proline;
d) the amino acid at position 337 is other than serine;
e) the amino acid position 363 is other than glutamic acid;
f) the amino acid at position 422 is other than glycine;
g) the amino acid at position 385 is other than leucine; and/or
h) the amino acid position 393 is other than an isoleucine,
and wherein said HPPD confers upon the plant increased herbicide tolerance as compared to a corresponding wild-type plant when expressed therein.

18. A seed produced by a transgenic plant comprising the transgenic plant cell of claim 13, wherein the seed is true breeding for an increased resistance to a coumarone-derivative herbicide as compared to a corresponding wild type seed.

19. A method of producing a transgenic plant cell having an increased resistance to a coumarone-derivative herbicide as compared to a corresponding wild type plant cell, comprising transforming a plant cell with an expression cassette comprising a mutated hydroxyphenyl pyruvate dioxygenase (mut-HPPD) nucleic acid.

20. A method of producing a transgenic plant comprising: (a) transforming a plant cell with an expression cassette comprising a mutated hydroxyphenyl pyruvate dioxygenase (mut-HPPD) nucleic acid, and (b) generating from the plant cell a plant with an increased resistance to coumarone-derivative herbicide relative to a corresponding wild type plant.

21. The method of claim 19, wherein the mut-HPPD nucleic acid comprises a polynucleotide sequence selected from the group consisting of:
a) the polynucleotide of SEQ ID NO: 1, 3 or 5, or a variant or derivative thereof;
b) the polynucleotide of SEQ ID NO: 7 or 9, or a variant or derivative thereof;
c) a polynucleotide encoding the polypeptide of SEQ ID NO: 2, 4, 6, 8, or 10, or a variant or derivative thereof;
d) a polynucleotide comprising at least 60 consecutive nucleotides of any of a) through c); and
e) a polynucleotide complementary to the polynucleotide of any of a) through d).

22. The method of claim 19, wherein the expression cassette further comprises a transcription initiation regulatory region and a translation initiation regulatory region that are functional in the plant.

23. A method of identifying or selecting a transformed plant cell, plant tissue, plant or part thereof comprising:
i) providing a transformed plant cell, plant tissue, plant or part thereof, wherein said transformed plant cell, plant tissue, plant or part thereof comprises the polynucleotide of SEQ ID NO: 1, 3 or 5, or a variant or derivative thereof, wherein the polynucleotide encodes a mutated hydroxyphenyl pyruvate dioxygenase (mut-HPPD) polypeptide that is used as a selection marker, and wherein said transformed plant cell, plant tissue, plant or part thereof may comprise a further isolated polynucleotide;
ii) contacting the transformed plant cell, plant tissue, plant or part thereof with at least one coumarine-derivative compound;
iii) determining whether the plant cell, plant tissue, plant or part thereof is affected by the inhibiting compound; and
iv) identifying or selecting the transformed plant cell, plant tissue, plant or part thereof.
